(19) United States
(12) Patent Application Publication
(10) Pub. No.: US 2023/0143215 A1
(43) Pub. Date: May 11, 2023

(54) IMMUNIZATION AGAINST SARS-COV-RELATED DISEASES

(71) Applicant: ISA Pharmaceuticals B.V., Oegstgeest (NL)

(72) Inventors: Cornelis Johannes Maria Melief, Oegstgeest (NL); Thomas Johannes Maria Beenakker, Oegstgeest (NL); Peter van Rijn, Oegstgeest (NL); Anna-Sophia Wiekmeijer, Oegstgeest (NL); Miranda Bernardina Johanna Molenaar, Oegstgeest (NL); Leonard Willem Frederik Hooftman, Oegstgeest (NL)

(73) Assignee: ISA Pharmaceuticals B.V., Oegstgeest (NL)

(21) Appl. No.: 17/920,416

(22) PCT Filed: Apr. 23, 2021
(86) PCT No.: PCT/EP2021/060688

§ 371 Date: Oct. 21, 2022

(30) Foreign Application Priority Data
Apr. 23, 2020 (EP) 20171203.1
Jan. 11, 2021 (EP) 21150889.0
Feb. 22, 2021 (EP) 21158319.0

(51) Int. Cl. C07K 14/005; A61K 39/215; A61P 31/14

(52) U.S. Cl. CPC C07K 14/005; A61K 39/215; A61P 31/14; A61K 39/00

(57) ABSTRACT

The invention provides long immunogenic peptides for treating diseases related to severe acute respiratory syndrome coronaviruses, such as SARS-CoV-2 or SARS-CoV-1. The invention also provides immunogenic compositions and vaccines comprises long immunogenic peptides of the invention and method of therapeutic treatment or prevention of SARS-CoV-related diseases comprising administration of immunogenic peptides according to the invention.

### FIELD OF THE INVENTION

The present invention relates to the field of immunology. In particular, it relates to novel immunogenic peptides, polynucleotides, compositions and methods for treating diseases related to severe acute respiratory syndrome coronaviruses, such as SARS-CoV-2 or SARS-CoV-1.

### BACKGROUND OF THE INVENTION

Coronaviruses are positive-sense single-stranded RNA viruses belonging to the family Coronaviridae. These viruses mostly infect animals, including birds and mammals. In humans, they often cause mild respiratory infections, such as those observed in the common cold. However, some recent human coronavirus infections have resulted in lethal endemics, which include the SARS (Severe Acute Respiratory Syndrome) and MERS (Middle East Respiratory Syndrome) endemics and the corona virus infectious disease 2019 (COVID-19) pandemic. All three outbreaks are caused by so called zoonotic coronaviruses that belong to the genus Betacoronavirus within Coronaviridae (Lu et al. (2020) Lancet 6736:1-10).

COVID-19 is caused by a new variant of the beta-coronavirus, named SARS-CoV-2. Not all infected patients with SARS-CoV-2 develop SARS. Most people infected with the coronavirus will experience mild to moderate respiratory illness and recover without requiring special treatment. However, elderly people, and those with underlying medical problems like cardiovascular disease, diabetes, chronic respiratory disease, obesity and cancer are more likely to develop serious illness. SARS-CoV-2 infection can be roughly divided into three stages: stage I, an asymptomatic incubation period with or without detectable virus; stage II, non-severe symptomatic period with the presence of virus; and stage III, severe respiratory symptomatic stage with high viral load (Shi et al. (2020) Cell Death Differ (https://doi.org/10.1038/s41418-020-0530-3)).

SARS-CoV variants have a genome size of ˜30 kilobases which, like other coronaviruses, encodes for multiple structural and non-structural proteins. The genome of SARS-CoV-2 (first reported in December 2019) shares almost 80% identity with SARS-CoV-1 (the first reported variant infecting human beings) (Zhou et al. (2020) Nature 579:270). The structural proteins include the spike (S) protein, the envelope (E) protein, the membrane (M) protein, and the nucleocapsid (N) protein.

SARS-CoV-1 was previously shown to bind to angiotensin-converting enzyme 2 (ACE2) for cell entry, mediated via the S protein (Gallagher & Buchmeier (2001) Virology 279:371; Li et al (2003) Nature 426:450). Recent studies reported that SARS-CoV-2 is also binding to ACE2 in vitro (Hoffmann et al (2020) Cell https://doi.org/10.1016/j.cell.2020.02.052; Walls et al (2020) Nature 531:114; Yan et al (2020) Science 367:1444; Zhou et al. (2020) Nature 579:270).

Vaccines that induce protection against infections with beta-coronaviruses, such as SARS-CoV-2 or SARS-CoV-1, have recently become available. The approved preventive vaccines so far all operate by the induction of virus-neutralizing antibodies.

### SUMMARY OF THE INVENTION

The present invention provides novel immunogenic long peptides based on the S, N and M proteins of SARS-CoV viruses. Furthermore, the invention provides immunogenic compositions and vaccines comprising the immunogenic peptides of the invention as well as methods of treatment (therapeutic treatment or prevention) of SARS-CoV-related diseases wherein the immunogenic peptides of the invention are administered to a subject in need thereof.

Accordingly, in a first main aspect, the invention relates to an immunogenic peptide comprising a fragment of a SARS-CoV protein, wherein said fragment is 20-40 amino acids in length and wherein said fragment comprises one or more of the sequences set forth in:

i) the amino acid sequence ALNTLVKQL (SEQ ID NO:52) (S protein),
ii) the amino acid sequence VLNDILSRL (SEQ ID NO:53) (S protein),
iii) the amino acid sequence LITGRLQSL (SEQ ID NO:54) (S protein),
iv) the amino acid sequence QLIRAAEIRASANLAATK (SEQ ID NO:55) (S protein),
v) the amino acid sequence RLNEVAKNL (SEQ ID NO:56) (S protein),
vi) the amino acid sequence FIAGLIAIV (SEQ ID NO:57) (S protein),
vii) the amino acid sequence AYRFNGIGVTQNVLY (SEQ ID NO:58) (S protein),
viii) the amino acid sequence FLPFFSNVTWF (SEQ ID NO:59) (S protein),
ix) the amino acid sequence LLALHRSYL (SEQ ID NO:60) (S protein),
x) the amino acid sequence VRFPNITNL (SEQ ID NO:61) (S protein),
xi) the amino acid sequence IYQTSNFRVQ (SEQ ID NO:62) (S protein),
xii) the amino acid sequence SIIAYTMSLG (SEQ ID NO:63) (S protein),
xiii) the amino acid sequence IVNNATNVVIK (SEQ ID NO:64) (S protein),
xiv) the amino acid sequence LPNNTASW (SEQ ID NO:65) (N protein),
xv) the amino acid sequence ALNTPKDHI (SEQ ID NO:66) (N protein),
xvi) the amino acid sequence LQLPQGTTL (SEQ ID NO:67) (N protein),
xvii) the amino acid sequence LALLLLDRLNQL (SEQ ID NO:68) (N protein),
xviii) the amino acid sequence GMSRIGMEV (SEQ ID NO:69) (N protein),
xix) the amino acid sequence FTALTQHGK (SEQ ID NO:70) (N protein),
xx) the amino acid sequence FPRGQGVPI (SEQ ID NO:71) (N protein),
xxi) the amino acid sequence YYRRATRRIRG (SEQ ID NO:72) (N protein),
xxii) the amino acid sequence LSPRWYFYY (SEQ ID NO:73) (N protein),
xxiii) the amino acid sequence LALLLLDRLNQL (SEQ ID NO:74) (N protein),
xxiv) the amino acid sequence GMSRIGMEV (SEQ ID NO:75) (N protein),
xxv) the amino acid sequence ILLNKHID (SEQ ID NO:76) (N protein),
xxvi) the amino acid sequence KTFPPTEPK (SEQ ID NO:77) (N protein),
xxvii) the amino acid sequence NVTQAFGRRGP (SEQ ID NO:78) (N protein),
xxviii) the amino acid sequence KHWPQIAQFA (SEQ ID NO:79) (N protein),
xxix) the amino acid sequence TLACFVLAAV (SEQ ID NO:80) (M protein),
xxx) the amino acid sequence SFNPETNIL (SEQ ID NO:81) (M protein),
xxxi) the amino acid sequence TVATSRTLSY (SEQ ID NO:82) (M protein),
xxxii) the amino acid sequence IAWNSNNLDSK (SEQ ID NO:83) (S protein RBD),
xxxiii) the amino acid sequence SKVGGNYNYLYRLFR (SEQ ID NO:84) (S protein RBD),
xxxiv) the amino acid sequence CNGVEGFNC (SEQ ID NO:85) (S protein RBD),
xxxv) the amino acid sequence NCYFPLQSYGFQPTNGVGY (SEQ ID NO:86) (S protein RBD),
xxxvi) the amino acid sequence GNYNYLYRLFRKSNL (SEQ ID NO:87) (S protein RBD)
xxxvii) the amino acid sequence set forth in SEQ ID NO:325 (S protein),
xxxviii) the amino acid sequence set forth in SEQ ID NO:331 (S protein RBD), or
xxxix) the amino acid sequence set forth in SEQ ID NO:340 (S protein RBD).

In a further aspect, the invention relates to a polynucleotide encoding an immunogenic peptide according to the invention. In an even further main aspect, the invention relates to an immunogenic composition comprising one or more immunogenic peptides according to the invention and a pharmaceutically-acceptable carrier.

In an even further main aspect, the invention relates to a method for the treatment, such as therapeutic treatment or prevention, of a SARS-CoV-related disease comprising administration to a human subject of one or more of the immunogenic peptides of the invention, such as 2 or more, e.g. 3 or more, such as 4 or more, e.g. 5 or more, such as 6 or more, e.g. 7 or more, such as 8 or more, e.g. 9 or more, such as 10 or more, e.g. 11 or more, such as 12 or more, e.g. 13, 14, 15, 16, 17, 18, 19, or 20 or more of the immunogenic peptides of the invention as defined herein.

The invention also relates to the design and production of immunogenic peptides and compositions based on virus variants.

### BRIEF DESCRIPTION OF THE FIGURES

FIG. 1: C57BL/6 mice vaccinated with pools of SLPs in Montanide, with standard regimen on day 0 and day 14. CD4+ and CD8+ T cell responses after overnight restimulation of splenocytes, isolated on day 21, with individual SLPs. Depicted are the percentage of cells with IFNγ production of CD4+ T cells (A) and CD8+ T cells (B) detected with ICS and FACS. Negative: non-stimulated cells, Control SLP: SLP derived from hepatitis B virus.

FIG. 2: CD4+ and CD8+ T cell responses after overnight restimulation of splenocytes, isolated on indicated days (7, 14 or 21), with individual SLPs. C57BL/6 mice were vaccinated with one pool of 6 SLPs in Montanide+CpG, with different regimens: single shot (ICS day 7), quick regimen (ICS day 14, with (black triangle) or without (black square) anti-OX40 at boost vaccination) and standard regimen (ICS day 21, with anti-OX40 at boost vaccination). Depicted are the percentage of cells with IFNγ production of CD4+ T cells (A) and CD8+ T cells (B) detected with ICS and FACS. Negative: non-stimulated cells, Control SLP: SLP derived from hepatitis B virus.

FIG. 3: Detection of antibodies to Spike protein after immunization with pools of SLPs, containing SLPs based on S antigen (i.e. S-SLPs in pool 1 & 2: SLP 10, SLP 12, SLP 15, SLP 30 and SLP 32; S-SLPs in pool 3 & 4: SLP 5, SLP 9, SLP 17, SLP 29, SLP 33). Serum of immunized mice was tested at different dilutions (X-axis). Two mice were immunized per group. Optical density was measured at 450 nm as measure for the presence of S-antigen binding IgG antibodies. Group 1: naïve, group 2: pool 1&2+CpG+OX40, group 3: pool 3&4+CpG+OX40.

FIG. 4: Spot Counts (SFU: spot forming units) per 1*10⁶ cells for each SLP, per subject tested. Convalescent donors are depicted as black circles and Control donors as black triangles. The control (HBV) SLP is at the far right.

FIG. 5: The predicted ligand counts of reference SLPs in a United Kingdom (UK) variant (B.1.1.7 lineage) background versus the predicted ligand counts of UK variant SLPs in a UK variant background (A), the predicted ligand counts of reference SLPs in a South-African (SA) variant (B.1.351 lineage) background versus the predicted ligand counts of SA variant SLPs in a SA variant background (B), the predicted ligand counts of reference SLPs in a Brazilian (BR) variant (P.1 lineage) background versus the predicted ligand counts of BR variant SLPs in a BR variant background and the predicted ligand counts of reference SLPs in an Indian (IN) variant (B.1.617 lineage) background versus the predicted ligand counts of BR variant SLPs in a BR variant background. The ligand counts are categorized in supertypes to capture the effect of replacing reference SLPs with variant SLPs for the relevant HLA-types at one glance. For example in (A), replacing SLP15 for SLP15-1 will result in a significant increase in A01 ligands when applied to a UK variant background, in (B) replacing SLP32 for SLP32-1 will result in a significant increase in A02 and B08 ligands when applied to a SA variant background, in (C) replacing SLP15 for SLP15-2 will result in a significant increase in A01 ligands when applied to a BR variant background and in (D) replacing SLP12 for SLP12-1 will result in a significant increase in A01 and A03 ligands when applied to a IN variant background.

### DETAILED DESCRIPTION OF THE INVENTION

### Definitions

The term “SARS-CoV”, when used herein, refers to a severe acute respiratory syndrome coronavirus, such as SARS-CoV-2 or SARS-CoV-1. The term “a SARS-CoV protein” refers to a protein of a SARS-CoV virus, such as the S (spike) protein, the N (nucleocapsid) protein or the M (membrane) protein.

When used herein, the term “immunogenic peptide” means a peptide capable of triggering or boosting an immune response. Likewise, the term “immunogenic composition” means a composition capable of triggering or boosting an immune response.

The term “a fragment of a SARS-CoV protein” refers to a sequence of consecutive amino acids that corresponds to, i.e. is identical to, a part of a SARS-CoV protein sequence. This does not exclude, however, that the fragment may be further modified, e.g. conjugated, such as covalently bound to another molecule, e.g. an adjuvant.

In the context of the present invention “20-40 amino acids in length” means that the number of amino acid residues is from 20 to 40 amino acids in length, i.e. 20, 21, 22, 23, 24, 25, 26, 27, 28, 29, 30, 31, 32, 33, 34, 35, 36, 37 38, 39 or 40 amino acid residues in length.

Peptides used in the invention, also denominated herein as long peptides, exceed the length of human leukocyte antigen (HLA) class I and class II presented epitopes. Preferably, the long peptides of the invention are synthetic peptides, denominated herein as synthetic long peptides (SLPs).

The term “corresponding” when used in connection with a sequence in the context of comparison of sequences, refers to the sequence with which a given sequence has the best alignment, as assessed with bioinformatic tools for alignment of sequences known in the art, such as BLAST.

When used herein, the term “variant background” refers to the protein sequence of the same target protein in a SARS-CoV-2 variant (e.g. the protein sequence of the S protein from B.1.1.7).

Unless specified otherwise, the terms “treatment” and “treating”, when used herein in the context of a medical intervention, include both therapeutic treatment as well as prevention (prophylactic treatment).

“Therapeutic treatment” refers to the administration of an effective amount of an immunogenic peptide, an immunogenic composition or vaccine with the purpose of easing, ameliorating, arresting or eradicating (curing) symptoms or disease states.

“Prevention” or “preventing” refers to the administration of an effective amount of an immunogenic peptide, an immunogenic composition or vaccine with the purpose of preventing a disease.

An “effective amount” refers to an amount effective, at dosages and for periods of time necessary, to achieve a desired therapeutic result.

A “SARS-CoV-related disease” refers to a disorder or disease that is related to an infection of a human subject with a SARS-CoV virus, such as SARS-CoV-1 or SARS-CoV-2. SARS-CoV-related diseases include respiratory diseases such as severe acute respiratory syndrome (SARS) or COVID-19, the symptoms of which may include fever, cough and shortness of breath, fatigue, muscle pain, diarrhea and sore throat. These diseases may lead to pneumonia, respiratory distress syndrome, cytokine release syndrome or multi-organ failure.

When used herein, the term “vaccine” means a product for triggering or boosting an immune response. A vaccine may be administered directly to a human subject or may be used in ex vivo immunization regimens. In ex vivo immunization regimens, the vaccine may be used to generate antigen-loaded antigen presenting cells (APCs), such as antigen-loaded activated Dendritic Cells (DCs), and subsequently stimulate expansion of antigen-specific T cells (e.g. CD4 and CD8 positive circulating T cells, Tumor Infiltrating Lymphocytes (TILs)). Such antigen-loaded APCs or expanded antigen-specific T cells are subsequently administered to a human subject. A vaccine may be a single immunogenic composition or comprise more than one immunogenic composition.

The term “skewing the T cell response towards immunogen-specific interferon-gamma producing CD4+ and CD8+ T cells” when used herein, refers to a modification of the immunogenic peptide, the immunogenic composition, vaccine or method of treatment of the invention, wherein the modification leads to a T cell immune response that is more skewed towards immunogen-specific interferon-gamma producing CD4+ and CD8+ T cells than in the absence of the modification. Such a modification may e.g. be the addition of an adjuvant. Thus, skewing of a response towards immunogen-specific interferon-gamma producing CD4+ and CD8+ T cells may be determined by comparing the relative presence of antigen-specific interferon-gamma producing CD4+ and CD8+ T cells after vaccination in a given number of Peripheral Blood Mononuclear Cells (PBMC) in the presence and absence of the modification, e.g. an adjuvant (see e.g. Scheiermann and Klinman (2014) Vaccine 32:6377). If there are higher levels of interferon-gamma producing antigen-specific CD4+ and CD8+ T cells in the presence than in the absence of the modification, the response is skewed towards antigen-specific interferon-gamma producing CD4+ and CD8+ T cells.

### Further Aspects and Embodiments of the Invention

As mentioned above, in a first main embodiment (Embodiment 1), the invention relates to an immunogenic peptide comprising a fragment of a SARS-CoV protein, wherein said fragment is 20-40 amino acids in length and wherein said fragment comprises one or more of the sequences set forth in:

i) the amino acid sequence ALNTLVKQL (SEQ ID NO:52) (S protein),
ii) the amino acid sequence VLNDILSRL (SEQ ID NO:53) (S protein),
iii) the amino acid sequence LITGRLQSL (SEQ ID NO:54) (S protein),
iv) the amino acid sequence QLIRAAEIRASANLAATK (SEQ ID NO:55) (S protein),
v) the amino acid sequence RLNEVAKNL (SEQ ID NO:56) (S protein),
vi) the amino acid sequence FIAGLIAIV (SEQ ID NO:57) (S protein),
vii) the amino acid sequence AYRFNGIGVTQNVLY (SEQ ID NO:58) (S protein),
viii) the amino acid sequence FLPFFSNVTWF (SEQ ID NO:59) (S protein),
ix) the amino acid sequence LLALHRSYL (SEQ ID NO:60) (S protein),
x) the amino acid sequence VRFPNITNL (SEQ ID NO:61) (S protein),
xi) the amino acid sequence IYQTSNFRVQ (SEQ ID NO:62) (S protein),
xii) the amino acid sequence SIIAYTMSLG (SEQ ID NO:63) (S protein),
xiii) the amino acid sequence IVNNATNVVIK (SEQ ID NO:64) (S protein),
xiv) the amino acid sequence LPNNTASW (SEQ ID NO:65) (N protein),
xv) the amino acid sequence ALNTPKDHI (SEQ ID NO:66) (N protein),
xvi) the amino acid sequence LQLPQGTTL (SEQ ID NO:67) (N protein),
xvii) the amino acid sequence LALLLLDRLNQL (SEQ ID NO:68) (N protein),
xviii) the amino acid sequence GMSRIGMEV (SEQ ID NO:69) (N protein),
xix) the amino acid sequence FTALTQHGK (SEQ ID NO:70) (N protein),
xx) the amino acid sequence FPRGQGVPI (SEQ ID NO:71) (N protein),
xxi) the amino acid sequence YYRRATRRIRG (SEQ ID NO:72) (N protein),
xxii) the amino acid sequence LSPRWYFYY (SEQ ID NO:73) (N protein),
xxiii) the amino acid sequence LALLLLDRLNQL (SEQ ID NO:74) (N protein),
xxiv) the amino acid sequence GMSRIGMEV (SEQ ID NO:75) (N protein),
xxv) the amino acid sequence ILLNKHID (SEQ ID NO:76) (N protein),
xxvi) the amino acid sequence KTFPPTEPK (SEQ ID NO:77) (N protein),
xxvii) the amino acid sequence NVTQAFGRRGP (SEQ ID NO:78) (N protein),
xxviii) the amino acid sequence KHWPQIAQFA (SEQ ID NO:79) (N protein),
xxix) the amino acid sequence TLACFVLAAV (SEQ ID NO:80) (M protein),
xxx) the amino acid sequence SFNPETNIL (SEQ ID NO:81) (M protein),
xxxi) the amino acid sequence TVATSRTLSY (SEQ ID NO:82) (M protein),
xxxii) the amino acid sequence IAWNSNNLDSK (SEQ ID NO:83) (S protein RBD),
xxxiii) the amino acid sequence SKVGGNYNYLYRLFR (SEQ ID NO:84) (S protein RBD),
xxxiv) the amino acid sequence CNGVEGFNC (SEQ ID NO:85) (S protein RBD),
xxxv) the amino acid sequence NCYFPLQSYGFQPTNGVGY (SEQ ID NO:86) (S protein RBD),
xxxvi) the amino acid sequence GNYNYLYRLFRKSNL (SEQ ID NO:87) (S protein RBD).

In another embodiment (Embodiment 2), said fragment comprises one or more of the sequences set forth in:

i) the amino acid sequence ALNTLVKQLSSN (SEQ ID NO:88) (S protein),
ii) the amino acid sequence VLNDILSRLDKV (SEQ ID NO:89) (S protein),
iii) the amino acid sequence LITGRLQSLQTY (SEQ ID NO:90) (S protein),
iv) the amino acid sequence QLIRAAEIRASANLAATK (SEQ ID NO:91) (S protein),
v) the amino acid sequence RLNEVAKNLNES (SEQ ID NO:92) (S protein),
vi) the amino acid sequence FIAGLIAIVMVT (SEQ ID NO:93) (S protein),
vii) the amino acid sequence AYRFNGIGVTQNVLY (SEQ ID NO:94) (S protein),
viii) the amino acid sequence FLPFFSNVTWFHAI (SEQ ID NO:95) (S protein),
ix) the amino acid sequence LLALHRSYLTPG (SEQ ID NO:96) (S protein),
x) the amino acid sequence VRFPNITNLCPF (SEQ ID NO:97) (S protein),
xi) the amino acid sequence IYQTSNFRVQPTE (SEQ ID NO:98) (S protein),
xii) the amino acid sequence SIIAYTMSLGAEN (SEQ ID NO:99) (S protein),
xiii) the amino acid sequence IVNNATNVVIKVCE (SEQ ID NO:100) (S protein),
xiv) the amino acid sequence LPNNTASWFTA (SEQ ID NO:101) (N protein),
xv) the amino acid sequence ALNTPKDHIGTR (SEQ ID NO:102) (N protein),
xvi) the amino acid sequence LQLPQGTTLPKG (SEQ ID NO:103) (N protein),
xvii) the amino acid sequence LALLLLDRLNQLESK (SEQ ID NO:104) (N protein),
xviii) the amino acid sequence GMSRIGMEVTPSGTWLTYT (SEQ ID NO:105) (N protein),
xix) the amino acid sequence FTALTQHGKEDL (SEQ ID NO:106) (N protein),
xx) the amino acid sequence FPRGQGVPINTN (SEQ ID NO:107) (N protein),
xxi) the amino acid sequence YYRRATRRIRGGDG (SEQ ID NO:108) (N protein),
xxii) the amino acid sequence LSPRWYFYYLGT (SEQ ID NO:109) (N protein),
xxiii) the amino acid sequence LALLLLDRLNQLESK (SEQ ID NO:110) (N protein),
xxiv) the amino acid sequence GMSRIGMEVTPS (SEQ ID NO:111) (N protein),
xxv) the amino acid sequence ILLNKHIDAYK (SEQ ID NO:112) (N protein),
xxvi) the amino acid sequence KTFPPTEPKKDK (SEQ ID NO:113) (N protein),
xxvii) the amino acid sequence NVTQAFGRRGPEQT (SEQ ID NO:114) (N protein),
xxviii) the amino acid sequence KHWPQIAQFAPSA (SEQ ID NO:115) (N protein),
xxix) the amino acid sequence TLACFVLAAVYRI (SEQ ID NO:116) (M protein),
xxx) the amino acid sequence SFNPETNILLNV (SEQ ID NO:117) (M protein),
xxxi) the amino acid sequence TVATSRTLSYYKL (SEQ ID NO:118) (M protein),
xxxii) the amino acid sequence IAWNSNNLDSKVGG (SEQ ID NO:119) (S protein RBD),
xxxiii) the amino acid sequence SKVGGNYNYLYRLFR (SEQ ID NO:120) (S protein RBD),
xxxiv) the amino acid sequence CNGVEGFNC (SEQ ID NO:121) (S protein RBD),
xxxv) the amino acid sequence NCYFPLQSYGFQPTNGVGY (SEQ ID NO:122) (S protein RBD),
xxxvi) the amino acid sequence GNYNYLYRLFRKSNL (SEQ ID NO:123) (S protein RBD).

In a further embodiment (Embodiment 3), said fragment comprises:

ii) the amino acid sequence VLNDILSRL and one or both of the amino acid sequences: SVLNDILSRL (SEQ ID NO:124) and SVLNDILSR (SEQ ID NO:125), or
iii) the amino acid sequence LITGRLQSL and one or more or all of the amino acid sequences: ITGRLQSLQTY (SEQ ID NO:126), RLITGRLQSLQ (SEQ ID NO:127), LITGRLQSLQ (SEQ ID NO:128) and GRLQSLQTY (SEQ ID NO:129), or
v) the amino acid sequence RLNEVAKNL and one or more or all of the amino acid sequences: RLNEVAKNLNE (SEQ ID NO:130), NLNESLIDL (SEQ ID NO:131), KEIDRLNEV (SEQ ID NO:132), and EVAKNLNESLI (SEQ ID NO:133), or
vi) the amino acid sequence FIAGLIAIV and one or more or all of the amino acid sequences: GLIAIVMV (SEQ ID NO:134), QYIKWPWYI (SEQ ID NO:135), QYIKWPWYIW (SEQ ID NO:136), KWPWYIWLGF (SEQ ID NO:137), QYIKWPWYIWL (SEQ ID NO:138), KYEQYIKW (SEQ ID NO:139), KWPWYIWL (SEQ ID NO:140), IWLGFIAGL (SEQ ID NO:141), YIKWPWYIW (SEQ ID NO:142), WPWYIWLGFIA (SEQ ID NO:143) and YEQYIKWPW (SEQ ID NO:144), or
vii) the amino acid sequence AYRFNGIGVTQNVLY and one or more or all of the amino acid sequences: LQIPFAMQM (SEQ ID NO:145), FAMQMAYRFNG (SEQ ID NO:146), QIPFAMQMAY (SEQ ID NO:147), IPFAMQMAY (SEQ ID NO:148), LQIPFAMQMAY (SEQ ID NO:149) and IPFAMQMAYRF (SEQ ID NO:150), or
viii) the amino acid sequence FLPFFSNVTWF and one or more or all of the amino acid sequences: VTWFHAIHV (SEQ ID NO:151), LFLPFFSNVTW (SEQ ID NO:152), STQDLFLPFFS (SEQ ID NO:153), LPFFSNVTWFH (SEQ ID NO:154), NVTWFHAIHVS (SEQ ID NO:155), NVTWFHAIHV (SEQ ID NO:156), LPFFSNVT (SEQ ID NO:157) and FLPFFSNV (SEQ ID NO:158), or
ix) the amino acid sequence LLALHRSYL and one or more or all of the amino acid sequences: TLLALHRSYLT (SEQ ID NO:159), NITRFQTLLAL (SEQ ID NO:160), TLLALHRSYL (SEQ ID NO:161), LPIGINITRFQ (SEQ ID NO:162), INITRFQTLL (SEQ ID NO:163), NITRFQTLLA (SEQ ID NO:164) and LPIGINIT (SEQ ID NO:165), or
x) the amino acid sequence VRFPNITNL and one or more or all of the amino acid sequences: IYQTSNFRVQ (SEQ ID NO:166), KGIYQTSNFRV (SEQ ID NO:167) and QPTESIVRFPN (SEQ ID NO:168), or
xi) the amino acid sequence IYQTSNFRVQ and one or both of the amino acid sequences: KGIYQTSNFRV (SEQ ID NO:169) and QPTESIVRFPN (SEQ ID NO:170), or
xii) the amino acid sequence SIIAYTMSLG and one or more or all of the amino acid sequences: SQSIIAYTMSL (SEQ ID NO:171), SVAYSNNSI (SEQ ID NO:172) and LGAENSVAYSN (SEQ ID NO:173), or
xiii) the amino acid sequence IVNNATNVVIK and one or more or all of the amino acid sequences: VIKVCEFQF (SEQ ID NO:174), NATNVVIKVCE (SEQ ID NO:175) and NVVIKVCEF (SEQ ID NO:176), or
xiv) the amino acid sequence LPNNTASW and one or more or all of the amino acid sequences: FTALTQHGK (SEQ ID NO:177), NTASWFTALTQ (SEQ ID NO:178), GLPNNTASWFT (SEQ ID NO:179) and LPNNTASWFT (SEQ ID NO:180), or
xv) the amino acid sequence ALNTPKDHI and one or both of the amino acid sequences: IIWVATEGA (SEQ ID NO:181) and ALNTPKDHIG (SEQ ID NO:182), or xvi) the amino acid sequence LQLPQGTTL and one or more or all of the amino acid sequences: GTTLPKGFY (SEQ ID NO:183), LQLPQGTTLPK (SEQ ID NO:184) and TTLPKGFYA (SEQ ID NO:185), or
xvii) the amino acid sequence LALLLLDRLNQL and one or both of the amino acid sequences: ALALLLLDRLN (SEQ ID NO:186) and RLNQLESKMSG (SEQ ID NO:187), or
xviii) the amino acid sequence GMSRIGMEVTPSGTWL and one or more or all of the amino acid sequences: VTPSGTWLTYT (SEQ ID NO:188), TWLTYTGAI (SEQ ID NO:189), EVTPSGTWLT (SEQ ID NO:190) and TPSGTWLTY (SEQ ID NO:191), or
xix) the amino acid sequence FTALTQHGK and one or more or all of the amino acid sequences: NTASWFTALTQ (SEQ ID NO:192), FPRGQGVPI (SEQ ID NO:193) and LPNNTASW (SEQ ID NO:194), or
xx) the amino acid sequence FPRGQGVPI and one or more or all of the amino acid sequences: NTASWFTALTQ (SEQ ID NO:195), FTALTQHGK (SEQ ID NO:196) and LPNNTASW (SEQ ID NO:197), or
xxi) the amino acid sequence YYRRATRRIRG and one or more or all of the amino acid sequences: SSPDDQIG (SEQ ID NO:198), IGYYRRATR (SEQ ID NO:199), GYYRRATRRIR (SEQ ID NO:200) and SPDDQIGYY (SEQ ID NO:201), or
xxii) the amino acid sequence LSPRWYFYY and one or more or all of the amino acid sequences: DLSPRWYFY (SEQ ID NO:202), DLSPRWYFYY (SEQ ID NO:203), GTGPEAGL (SEQ ID NO:204), MKDLSPRWYFY (SEQ ID NO:205), KDLSPRWYFY (SEQ ID NO:206), LSPRWYFYYL (SEQ ID NO:207), YLGTGPEA (SEQ ID NO:208), KMKDLSPRW (SEQ ID NO:209), KDLSPRWYFYY (SEQ ID NO:210), KMKDLSPRWY (SEQ ID NO:211), KMKDLSPRWYF (SEQ ID NO:212), KDLSPRWYF (SEQ ID NO:213) and SPRWYFYYLGT (SEQ ID NO:214), or
xxiii) the amino acid sequence LALLLLDRLNQL and one or both of the amino acid sequences: ALALLLLDRLN (SEQ ID NO:215) and RLNQLESKMSG (SEQ ID NO:216), or
xxiv) the amino acid sequence GMSRIGMEV and one or more or all of the amino acid sequences: VTPSGTWLTYT (SEQ ID NO:217), EVTPSGTWL (SEQ ID NO:218), GMSRIGMEVTP (SEQ ID NO:219), EVTPSGTWLT (SEQ ID NO:220), TPSGTWLTY (SEQ ID NO:221) and MEVTPSGTWL (SEQ ID NO:222), or
xxv) the amino acid sequence ILLNKHID and one or more or all of the amino acid sequences: LLNKHIDAY (SEQ ID NO:223), KTFPPTEPKKD (SEQ ID NO:224), LLNKHIDAYK (SEQ ID NO:225), KTFPPTEP (SEQ ID NO:226), KHIDAYKTF (SEQ ID NO:227) and VILLNKHIDAY (SEQ ID NO:228), or
xxvi) the amino acid sequence KTFPPTEPK and one or more or all of the amino acid sequences: LLNKHIDAY (SEQ ID NO:229), KTFPPTEPKKD (SEQ ID NO:230), LLNKHIDAYK (SEQ ID NO:231), KHIDAYKTF (SEQ ID NO:232), VILLNKHIDAY (SEQ ID NO:233), and ILLNKHID (SEQ ID NO:234), or
xxviii) the amino acid sequence KHWPQIAQFA and one or both of the amino acid sequences: QFAPSASA (SEQ ID NO:235) and RQGTDYKHW (SEQ ID NO:236), or xxix) the amino acid sequence TLACFVLAAV and one or both of the amino acid sequences: TLACFVLA (SEQ ID NO:237), WLLWPVTLA (SEQ ID NO:238), FVLAAVYRI (SEQ ID NO:239), LWLLWPVTL (SEQ ID NO:240), LWPVTLACF (SEQ ID NO:241), ACFVLAAVYRI (SEQ ID NO:242) and WPVTLACFVL (SEQ ID NO:243), or
xxx) the amino acid sequence SFNPETNIL and one or both of the amino acid sequences: SMWSFNPETN (SEQ ID NO:244), FNPETNILL (SEQ ID NO:245), LFARTRSMW (SEQ ID NO:246), LFARTRSMWSF (SEQ ID NO:247), RTRSMWSFNP (SEQ ID NO:248), ARTRSMWSFNP (SEQ ID NO:249), FARTRSMW (SEQ ID NO:250) and FARTRSMWSF (SEQ ID NO:251), or
xxxi) the amino acid sequence TVATSRTLSY and one or both of the amino acid sequences: VATSRTLSYY (SEQ ID NO:252), ATSRTLSYYKL (SEQ ID NO:253), ITVATSRTLSY (SEQ ID NO:254), VATSRTLSY (SEQ ID NO:255), TVATSRTLSYY (SEQ ID NO:256), ATSRTLSYYK (SEQ ID NO:257), ATSRTLSY (SEQ ID NO:258), VATSRTLSYYK (SEQ ID NO:259), TSRTLSYYKLG (SEQ ID NO:260), SRTLSYYKLGA (SEQ ID NO:261), ITVATSRTL (SEQ ID NO:262) and LPKEITVATSR (SEQ ID NO:263), or
xxxii) the amino acid sequence IAWNSNNLDSK, and one or more of all of the amino acid sequences: GCVIAWNSNNLDSKV (SEQ ID NO:264), KVGGNYNYLYRLFRK (SEQ ID NO:265), VGGNYNYLYRLFRKS (SEQ ID NO:266), GGNYNYLYRLFRKSN (SEQ ID NO:267), KVGGNYNYLY (SEQ ID NO:268), VIAWNSNNL (SEQ ID NO:269), KVGGNYNYL (SEQ ID NO:270), NYNYLYRLF (SEQ ID NO:271), SKVGGNYNYLY (SEQ ID NO:272), VGGNYNYLY (SEQ ID NO:273), NYNYLYRLFR (SEQ ID NO:274), YNYLYRLFR (SEQ ID NO:275) and YLYRLFRKS (SEQ ID NO:276), or
xxxiii) the amino acid sequence SKVGGNYNYLYRLFR, and one or more of all of the amino acid sequences: KVGGNYNYLYRLFRK (SEQ ID NO:277), VGGNYNYLYRLFRKS (SEQ ID NO:278), GGNYNYLYRLFRKSN (SEQ ID NO:279), GNYNYLYRLFRKSNL (SEQ ID NO:280), NYNYLYRLFRKSNLK (SEQ ID NO:281), GCVIAWNSNNLDSKV (SEQ ID NO:282), KVGGNYNYLY (SEQ ID NO:283), KVGGNYNYL (SEQ ID NO:284), NYNYLYRLF (SEQ ID NO:285), SKVGGNYNYLY (SEQ ID NO:286), VGGNYNYLY (SEQ ID NO:287), NYNYLYRLFR (SEQ ID NO:288), YNYLYRLFR (SEQ ID NO:289), YLYRLFRKS (SEQ ID NO:290) and VIAWNSNNL (SEQ ID NO:291), or
xxxiv) the amino acid sequence CNGVEGFNC, and one or more of all of the amino acid sequences: NGVEGFNCYFPLQSY (SEQ ID NO:292), GVEGFNCYFPLQSYG (SEQ ID NO:293), VEGFNCYFPLQSYGF (SEQ ID NO:294), EGFNCYFPLQSYGFQ (SEQ ID NO:295), GFNCYFPLQSYGFQP (SEQ ID NO:296), CYFPLQSY (SEQ ID NO:297), YFPLQSYGFQP (SEQ ID NO:298), NCYFPLQSYGF (SEQ ID NO:299) and FPLQSYGFQP (SEQ ID NO:300), or
xxxv) the amino acid sequence NCYFPLQSYGFQPTNGVGY and one or more of all of the amino acid sequences: NGVEGFNCYFPLQSY (SEQ ID NO:301), GVEGFNCYFPLQSYG (SEQ ID NO:302), VEGFNCYFPLQSYGF (SEQ ID NO:303), EGFNCYFPLQSYGFQ (SEQ ID NO:304) and GFNCYFPLQSYGFQP (SEQ ID NO:305), or
xxxvi) the amino acid sequence GNYNYLYRLFRKSNL and one or more of all of the amino acid sequences: KVGGNYNYLYRLFRK (SEQ ID NO:306), VGGNYNYLYRLFRKS (SEQ ID NO:307), GGNYNYLYRLFRKSN (SEQ ID NO:308), GNYNYLYRLFRKSNL (SEQ ID NO:309), NYNYLYRLFRKSNLK (SEQ ID NO:310), YNYLYRLFRKSNLKP (SEQ ID NO:311), NYLYRLFRKSNLKPF (SEQ ID NO:312), YLYRLFRKSNLKPFE (SEQ ID NO:313), LYRLFRKSNLKPFER (SEQ ID NO:314), YRLFRKSNLKPFERD (SEQ ID NO:315), KVGGNYNYLY (SEQ ID NO:316), KVGGNYNYL (SEQ ID NO:317), RLFRKSNLKP (SEQ ID NO:318), SKVGGNYNYLY (SEQ ID NO:319), VGGNYNYLY (SEQ ID NO:320), KSNLKPFER (SEQ ID NO:321) and YRLFRKSNLKP (SEQ ID NO:322).

In a further embodiment, the invention relates to an immunogenic peptide comprising a fragment of a SARS-CoV protein of less than 40 amino acids in length and wherein said fragment comprises one or more of the sequences set forth in: SEQ ID NO:325, SEQ ID NO:331 or SEQ ID NO:340.

In one embodiment, said peptide consists of said fragment of a SARS-CoV protein.

In one embodiment, said peptide is 20-40 amino acids in length, such as 20, 21, 22, 23, 24, 25, 26, 27, 28, 29, 30, 31, 32, 33, 34, 35, 36, 37, 38, 39 or 40 amino acids length, e.g. 20-34 amino acids in length, such as 20-33 amino acids in length, e.g. 20-32 amino acids in length, such as 20-31 amino acids in length, e.g. 20-30 amino acids in length, such as 20-29 amino acids in length, e.g. 20-28 amino acids in length, such as 20-27 amino acids in length, e.g. 20-26 or 20-25 amino acids in length.

In one embodiment, fragment is 20-40 amino acids in length, such as 20, 21, 22, 23, 24, 25, 26, 27, 28, 29, 30, 31, 32, 33, 34, 35, 36, 37, 38, 39 or 40 amino acids length, e.g. 20-34 amino acids in length, such as 20-33 amino acids in length, e.g. 20-32 amino acids in length, such as 20-32 amino acids in length, such as 20-31 amino acids in length, e.g. 20-30 amino acids in length, such as 20-29 amino acids in length, e.g. 20-28 amino acids in length, such as 20-27 amino acids in length, e.g. 20-26 or 20-25 amino acids in length.

In one embodiment, the peptide comprises:

a) the amino acid sequence ALNTLVKQLSNN and the amino acid sequence VLNDILSRLDKV, or
b) the amino acid sequence VLNDILSRLDKV and the amino acid sequence LITGRLQSLQTY.

In one embodiment, the peptide comprises or consists of a sequence selected from the group consisting of: SEQ ID NO:1-45 or SEQ ID NO:326 or SEQ ID NO:327 or SEQ ID NO:328 or SEQ ID NO:330 or SEQ ID NO:342 or SEQ ID NO:343.

In a further embodiment, the immunogenic peptide comprises or consist of a sequence selected from the group consisting of: SEQ ID NO: 5, 9, 10, 12, 15, 17, 19, 22, 24, 29, 30, 32, 33, 36, 37, 38, 39, 40, 44 and 45. In another embodiment, the immunogenic peptide comprises or consist of a sequence selected from the group consisting of: SEQ ID NO: 9, 10, 12, 15, 19, 22, 32, 33, 37, 38, 39 and 40. In a further embodiment, the immunogenic peptide comprises or consist of a sequence selected from the group consisting of: SEQ ID NO: 9, 19, 32, 33, 38 and 39. In a further embodiment, the immunogenic peptide comprises or consist of a sequence selected from the group consisting of: SEQ ID NO: 326, 19, 32, 33, 38 and 39. In a further embodiment, the immunogenic peptide comprises or consist of a sequence selected from the group consisting of: SEQ ID NO: 9, 19, 325, 33, 38 and 39. In an even further embodiment, the immunogenic peptide comprises or consist of a sequence selected from the group consisting of: SEQ ID NO: 19, 22 and 38. In a further embodiment, the immunogenic peptide comprises or consists of the sequence set forth in SEQ ID NO:323.

### Peptides Used in the Compositions and Methods of the Invention

Preferably, the immunogenic peptides of the invention are capable of inducing a potent combined antigen-directed CD4+T helper and CD8+ cytotoxic T cell response, when administered to a human subject. The peptides may be predicted to be immunogenic and/or may be proven to be immunogenic using in vitro or ex vivo assays or by doing in vivo tests appreciated in the art to establish immunogenicity. Preferably, the peptides can be used effectively in the prevention, partial clearance and/or treatment or full clearance of a SARS-CoV-related disease or condition in a subject, preferably as detectable by:
activation or an induction of the immune system and/or an increase in antigen-specific activated CD4+ and/or CD8+ T-cells in peripheral blood or in tissues as established by interferon-gamma ELISpot assay or by tetramer staining of CD4+ or CD8+ T cells or an increase of the cytokines (such as interferon-gamma, TNF-alpha, interleukin-2) produced by these T-cells as established by intracellular cytokine staining of CD4+ and CD8+ T cells in flow cytometry after at least one week of treatment; and/or
activation of an anti-viral antibody response with preferably virus-neutralizing capacity and demonstrable in serum/plasma by ELISA and/or virus neutralization assay and/or
activation of B cells and resulting (neutralizing) virus-specific antibody production of different immunoglobulin subclasses, including, but not limited to IgG and IgA.

In a preferred embodiment, the vaccine or (an) immunogenic composition(s) of the invention or used in the method of the invention comprise(s) a combination of peptides wherein said combination of peptides comprises epitopes capable of binding to at least 70%, 80%, 90%, 92%, 94%, 95%, 96%, 97%, 98%, 99% or 100% of the HLA class I molecules that are encoded by HLA alleles predominant in the population of human subjects to be treated. Preferred HLA class I epitopes in peptides according to the invention are epitopes capable of binding to: HLA-A0101; HLA-A0201; HLA-A0206; HLA-A0301; HLA-A1101; HLAA2301; HLA-A2402; HLA-A2501; HLA-A2601; HLA-A2902; HLA-A3001; HLAA3002; HLA-A3101; HLA-A3201; HLA-A3303; HLA-A6801; HLA-A6802; HLAA7401; HLA-B0702; HLA-B0801; HLA-B1301; HLA-B1302; HLA-B1402; HLAB1501; HLA-B1502; HLA-B1525; HLA-B1801; HLA-B2702; HLA-B2705; HLAB3501; HLA-B3503; HLA-B3701; HLA-B3801; HLA-B3901; HLA-B4001; HLAB4002; HLA-B4402; HLA-B4403; HLA-B4601; HLA-B4801; HLA-B4901; HLAB5001; HLA-B5101; HLA-B5201; HLA-B5301; HLA-B5501; HLA-B5601; HLAB5701; HLA-B5801 and HLA-B5802. In a preferred embodiment, the vaccine or (an) immunogenic composition(s) of the invention or used in the method of the invention comprise(s) a combination of peptides wherein said combination of peptides comprises epitopes capable of binding to at least 70%, 80%, 90%, 92%, 94%, 95%, 96%, 97%, 98%, 99% or 100% of the HLA class I and epitopes capable of binding to at least 20%, 30%, 40%, 42%, 44%, 45%, 46%, 47%, 48%, 49% or 50% of the HLA class II molecules that are encoded by HLA alleles predominant in the population of human subjects to be treated. In a preferred embodiment, a peptide used in the invention comprises a CTL epitope that shows binding affinity, preferably at least intermediate binding affinity, more preferably high binding affinity to an HLA class I molecules that is encoded by an HLA allele predominant in the population of human subjects to be treated. Preferably, a peptide used in the invention comprises a CTL epitope that shows binding affinity, preferably at least intermediate binding affinity, more preferably high binding affinity to at least one HLA class I molecule of the group of HLA class I molecules consisting of: HLA-A0101; HLA-A0201; HLA-A0206; HLA-A0301; HLA-A1101; HLA-A2301; HLA-A2402; HLA-A2501; HLA-A2601; HLA-A2902; HLA-A3001; HLA-A3002; HLA-A3101; HLA-A3201; HLA-A3303; HLA-A6801; HLA-A6802; HLA-A7401; HLA-B0702; HLA-B0801; HLA-B1301; HLA-B1302; HLA-B1402; HLA-B1501; HLA-B1502; HLA-B1525; HLA-B1801; HLA-B2702; HLA-B2705; HLA-B3501; HLA-B3503; HLA-B3701; HLA-B3801; HLA-B3901; HLA-B4001; HLA-B4002; HLA-B4402; HLA-B4403; HLA-B4601; HLA-B4801; HLA-B4901; HLA-B5001; HLA-B5101; HLA-B5201; HLA-B5301; HLA-B5501; HLA-B5601; HLA-B5701; HLA-B5801 and HLA-B5802.

In a preferred embodiment, an immunogenic peptide of the invention comprises a CTL epitope as described above and a T helper epitope that shows binding affinity, preferably at least intermediate binding affinity, more preferably high binding affinity to an HLA class II molecules that is encoded by an HLA allele predominant in the population of human subjects to be treated.

In a preferred embodiment, immunogenic peptides of the invention do not have a cysteine residue at the N- or C-terminus of the peptide. Furthermore, in another preferred embodiment, immunogenic peptides of the invention do not comprise more than two cysteine residues. In another preferred embodiment, immunogenic peptides of the invention do not comprise more than three methionines. In another preferred embodiment, immunogenic peptides of the invention do not have a glutamine at the N-terminus.

Preferably, an immunogenic peptide of the invention is an isolated peptide, wherein “isolated” does not reflect the extent to which the peptide is purified, but indicates that the peptide has been removed from its natural milieu (i.e., that has been subject to human manipulation), and may be a recombinantly produced peptide or a synthetically produced peptide.

The immunogenic peptide of the invention may not or may comprise a non-naturally occurring sequence as a result of comprising additional amino acids (N-terminal or C-terminal of the SARS-CoV protein fragment) not originating from the SARS-CoV protein antigen and/or as a result of comprising a modified amino acid and/or a non-naturally occurring amino acid and/or a covalently linked functional group such as a fluorinated group, a fluorocarbon group, a human toll-like receptor ligand and/or agonist, an oligonucleotide conjugate, PSA, a sugar chains or glycan, a Pam3cys and/or derivative thereof, preferably such as described in WO2013051936A1, CpG oligodeoxynucleotides (CpG-ODNs), cyclic dinucleotides (CDNs), a DC pulse cassette, a tetanus toxin derived peptide or a human HMGB1 derived peptide. The peptide of the invention may comprise aminobutyric acid (Abu, an isostere of cysteine). A cysteine of the peptide of the invention may be replaced by Abu. Peptides are typically produced synthetically. This may be done by solid phase peptide synthesis or by any other suitable method.

In a further aspect, the invention provides a polynucleotide encoding an immunogenic peptide of the invention as defined herein. A polynucleotide may be any polynucleotide comprising e.g. RNA, DNA and/or cDNA and may comprise nucleotide analogues and/or nucleotide equivalents such as a peptide nucleic acid or a morpholino nucleotide analogue. A polynucleotide may be codon-optimized for a host of choice to facilitate expression of the encoded peptide or polypeptide.

The polynucleotide according to the invention does not encode a full-length SARS-CoV protein, but rather encodes an immunogenic peptide according to the invention, as such, or flanked by amino acid sequences that are not contiguous with the SARS-CoV protein from which the immunogenic peptide is derived. Thus, in the polynucleotide of the invention, the sequence encoding the immunogenic peptide may be part of a larger open reading frame also containing flanking amino acids, provided that such flanking amino acids are not contiguous with the immunogenic peptide sequence in the SARS-CoV protein from which the immunogenic peptide is derived. Such flanking amino acids may for example be from proteins other than a SARS-CoV protein and/or they may be from other locations within a SARS-CoV protein that are not contiguous with the peptide they flank. Preferably, the polynucleotide encodes two or more immunogenic peptides according to the invention arranged as “beads-on-a-string”, whereby the peptides according to the invention (the “beads”) are linked directly together and/or are linked through linker sequences that are from proteins other than a SARS-CoV protein and/or from other locations within SARS-CoV that are not contiguous with the peptide they flank. The amino acid sequences flanking or linking the peptides may comprise proteolytic cleavage sites.

A polynucleotide according to the invention may be applied to deliver a peptide according to the invention in various ways. A polynucleotide according to the invention may e.g. be used in the production of recombinant protein or peptide in a suitable host cell (e.g. a bacterial host cell such as E. coli, a suitable yeast host cell such as S. cerevisiae, a suitable filamentous fungal such as an Aspergillus or mammalian host cell) from which the recombinant protein or peptide may be purified. Alternatively, the polynucleotide may be operably linked to expression regulatory sequences (promoters and the like) and incorporated in an expression construct for human cells. Such (autologous) cells may be transfected or transduced ex vivo to be (re)-administered to a subject in need thereof. Alternatively, such expression construct according to the invention may be incorporated into a suitable gene therapy vector. Suitable viral expression constructs include e.g. vectors that are based on adenovirus, adeno-associated virus (AAV), retroviruses or modified vaccinia Ankara (MVA). The polynucleotide according to the invention may also be operably linked to a sequence encoding and adjuvant such as a Toll-like receptor (TLR) ligand, a NOD ligand, or a RIG-I ligand.

In a further aspect, the present invention provides a cell comprising the polynucleotide according to the invention. Such a cell can be used for e.g. production of an immunogenic peptide according to the invention or for medical purposes such as prevention and/or treatment of a SARS-CoV related disease as defined elsewhere herein. Said cell may be any host cell.

### Further Methods of Treatment, Immunogenic Compositions and Vaccines

### Methods of Treatment

In a further main aspect, the invention relates to a method for the treatment (therapeutic treatment or prevention) of a SARS-CoV-related disease, comprising

(i) administering to a human subject (or immunizing a human subject with) one or more immunogenic peptides of the invention, and/or
(ii) administering to a human subject a population of antigen-loaded activated antigen presenting cells (APCs) or a population of expanded antigen-specific T cells, wherein said population of cells has been generated ex vivo using a plurality of peptides.

Similarly, in a main aspect, the invention relates to a method for the treatment, such as therapeutic treatment or prevention, of a SARS-CoV-related disease comprising administration to a human subject of one or more of the immunogenic peptides of the invention, such as 2 or more, e.g. 3 or more, such as 4 or more, e.g. 5 or more, such as 6 or more, e.g. 7 or more, such as 8 or more, e.g. 9 or more, such as 10 or more, e.g. 11 or more, such as 12 or more, e.g. 13, 14, 15, 16, 17, 18, 19, or 20 or more of the immunogenic peptides of the invention.

Similarly, in a further aspect, the invention relates to one or more of the immunogenic peptides of the invention, such as 2 or more, e.g. 3 or more, such as 4 or more, e.g. 5 or more, such as 6 or more, e.g. 7 or more, such as 8 or more, e.g. 9 or more, such as 10 or more, e.g. 11 or more, such as 12 or more, e.g. 13, 14, 15, 16, 17, 18, 19, or 20 or more of the immunogenic peptides of the invention, for use in the treatment, such as therapeutic treatment or prevention, of a SARS-CoV-related disease.

In one embodiment, the method comprises administration of 2 or more, e.g. 3 or more, such as 4 or more, e.g. 5 or more, such as 6 or more, e.g. 7 or more, such as 8 or more, e.g. 9 or more, such as 10 or more, e.g. 11 or more, such as 12 or more of the immunogenic peptides defined in embodiment 1(i) to 1(xxxi) or of the immunogenic peptides defined in embodiment 2 (i) to 2 (xxxi), or of the immunogenic peptides defined in embodiment 3.

In another embodiment, the method comprises administration of:
the immunogenic peptide defined in embodiment 1 (i) and the immunogenic peptide defined in embodiment 1 (ii), or
the immunogenic peptide defined in embodiment 1 (i) and the immunogenic peptide defined in embodiment 1 (v), or
the immunogenic peptide defined in embodiment 1 (i) and the immunogenic peptide defined in embodiment 1 (xvi), or
the immunogenic peptide defined in embodiment 1 (ii) and the immunogenic peptide defined in embodiment 1 (v), or
the immunogenic peptide defined in embodiment 1 (ii) and the immunogenic peptide defined in embodiment 1 (xvi), or
the immunogenic peptide defined in embodiment 1 (v) and the immunogenic peptide defined in embodiment 1 (xvi), or
the immunogenic peptide defined in embodiment 1 (i), the immunogenic peptide defined in embodiment 1 (ii) and the immunogenic peptides defined in embodiment 1 (v), or
the immunogenic peptide defined in embodiment 1 (i), the immunogenic peptide defined in embodiment 1 (ii) and the immunogenic peptides defined in embodiment 1 (xvi), or
the immunogenic peptide defined in embodiment 1 (i), the immunogenic peptide defined in embodiment 1 (v) and the immunogenic peptides defined in embodiment 1 (xvi), or
the immunogenic peptide defined in embodiment 1 (ii), the immunogenic peptide defined in embodiment 1 (v) and the immunogenic peptides defined in embodiment 1 (xvi), or
the immunogenic peptide defined in embodiment 1 (i), the immunogenic peptide defined in embodiment 1 (ii), the immunogenic peptides defined in embodiment 1 (v), and the immunogenic peptides defined in embodiment 1 (xvi), or
the immunogenic peptide defined in embodiment 1 (xix) and the immunogenic peptide defined in embodiment 1 (xx), or
the immunogenic peptide defined in embodiment 1 (xix) and the immunogenic peptide defined in embodiment 1 (viii), or
the immunogenic peptide defined in embodiment 1 (xix) and the immunogenic peptide defined in embodiment 1 (xxxiv), or
the immunogenic peptide defined in embodiment 1 (xx) and the immunogenic peptide defined in embodiment 1 (viii), or
the immunogenic peptide defined in embodiment 1 (xx) and the immunogenic peptide defined in embodiment 1 (xxxiv), or
the immunogenic peptide defined in embodiment 1 (viii) and the immunogenic peptide defined in embodiment 1 (xxxiv), or
the immunogenic peptide defined in embodiment 1 (xix), the immunogenic peptide defined in embodiment 1 (xx) and the immunogenic peptides defined in embodiment 1 (viii), or
the immunogenic peptide defined in embodiment 1 (xix), the immunogenic peptide defined in embodiment 1 (xx) and the immunogenic peptides defined in embodiment 1 (xxxiv), or
the immunogenic peptide defined in embodiment 1 (xix), the immunogenic peptide defined in embodiment 1 (viii) and the immunogenic peptides defined in embodiment 1 (xxxiv), or
the immunogenic peptide defined in embodiment 1 (xx), the immunogenic peptide defined in embodiment 1 (viii) and the immunogenic peptides defined in embodiment 1 (xxxiv), or
the immunogenic peptide defined in embodiment 1 (xix), the immunogenic peptide defined in embodiment 1 (xx), the immunogenic peptides defined in embodiment 1 (viii), and the immunogenic peptides defined in embodiment 1 (xxxiv).

In another embodiment, the method comprises administration of:
an immunogenic peptide as defined in embodiment 1 (xxx) and an immunogenic peptide defined in embodiment 1 (xxii), or
an immunogenic peptide as defined in embodiment 1 (xxx) and an immunogenic peptide as defined in embodiment 1 (xxviii), or
an immunogenic peptide as defined in embodiment 1 (xxii) and an immunogenic peptide as defined in embodiment 1 (xxviii), or
an immunogenic peptide as defined in embodiment 2 (xxx) and an immunogenic peptide as defined in embodiment 2 (xxii), or
an immunogenic peptide as defined in embodiment 2 (xxx) and an immunogenic peptide as defined in embodiment 2 (xxviii), or
an immunogenic peptide as defined in embodiment 2 (xxii) and an immunogenic peptide as defined in embodiment 2 (xxviii), or
an immunogenic peptide as defined in embodiment 3 (xxx) and an immunogenic peptide as defined in embodiment 3 (xxii), or
an immunogenic peptide as defined in embodiment 3 (xxx) and an immunogenic peptide as defined in embodiment 3 (xxviii), or
an immunogenic peptide as defined in embodiment 3 (xxii) and an immunogenic peptide as defined in embodiment 3 (xxviii), or
an immunogenic peptide as defined in embodiment 1 (xxx), an immunogenic peptide as defined in embodiment 1 (xxii), and an immunogenic peptide as defined in embodiment 1 (xxviii), or
an immunogenic peptide as defined in embodiment 2 (xxx), an immunogenic peptide as defined in embodiment 2 (xxii), and an immunogenic peptide as defined in embodiment 2 (xxviii), or
an immunogenic peptide as defined in embodiment 3 (xxx), an immunogenic peptide as defined in embodiment 3 (xxii), and an immunogenic peptide as defined in embodiment 3 (xxviii), or
two, three, four, five, six or all seven of: the immunogenic peptides as defined in embodiment 1 (ii), embodiment 1 (xxxiii), embodiment 1 (xxx), embodiment 1 (ix), embodiment 1 (x), embodiment 1 (xxi) and embodiment 1 (xxii), or
two, three, four, five, six or all seven of: the immunogenic peptides as defined in embodiment 2 (ii), embodiment 2 (xxxiii), embodiment 2 (xxx), embodiment 2 (ix), embodiment 2 (x), embodiment 2 (xxi) and embodiment 2 (xxii), or
two, three, four, five, six or all seven of: the immunogenic peptides as defined in embodiment 3 (ii), embodiment 3 (xxxiii), embodiment 3 (xxx), embodiment 3 (ix), embodiment 3 (x), embodiment 3 (xxi) and embodiment 3 (xxii), or
two, three, four, five, six, seven or all eight of: the immunogenic peptides as defined in embodiment 1 (ii), embodiment 1 (xxxiii), embodiment 1 (xxx), embodiment 1 (ix), embodiment 1 (x), embodiment 1 (xxi), embodiment 1 (xxii) and embodiment 1 (xxviii), or
two, three, four, five, six, seven or all eight of: the immunogenic peptides as defined in embodiment 2 (ii), embodiment 2 (xxxiii), embodiment 2 (xxx), embodiment 2 (ix), embodiment 2 (x), embodiment 2 (xxi), embodiment 2 (xxii) and embodiment 2 (xxviii), or
two, three, four, five, six, seven or all eight of: the immunogenic peptides as defined in embodiment 3 (ii), embodiment 3 (xxxiii), embodiment 3 (xxx), embodiment 3 (ix), embodiment 3 (x), embodiment 3 (xxi), embodiment 3 (xxii) and embodiment 3 (xxviii).

In another embodiment, the method comprises administration of the peptides set forth in:
SEQ ID NO:19 and SEQ ID NO:38, or
SEQ ID NO:19 and SEQ ID NO:323, or
SEQ ID NO:38 and SEQ ID NO:323, or
SEQ ID NO:19, SEQ ID NO:38 and SEQ ID NO:323, or
two, three or all of SEQ ID NO:19, 22, 38 and 323, or
two, three, four, five, six or all of: SEQ ID NO:9, 12, 19, 32, 33, 37 and 38 (preferably including at least SEQ ID NO:19 and 38), or
two, three, four, five, six or all of: SEQ ID NO:326, 12, 19, 32, 33, 37 and 38 (preferably including at least SEQ ID NO:19 and 38), or
two, three, four, five, six or all of: SEQ ID NO:9, 12, 19, 325, 33, 37 and 38 (preferably including at least SEQ ID NO:19 and 38), or
two, three, four, five, six or all of: SEQ ID NO:9, 12, 19, 32, 33, 328 and 38 (preferably including at least SEQ ID NO:19 and 38), or
two, three, four, five, six or all of: SEQ ID NO:9, 340, 19, 32, 33, 37 and 38 (preferably including at least SEQ ID NO:19 and 38), or
two, three, four, five, six, seven or all of: SEQ ID NO:9, 12, 19, 32, 33, 37, 38 and 323 (preferably including at least SEQ ID NO:19 and 38), or
two, three, four, five, six, seven or all of: SEQ ID NO:326, 12, 19, 32, 33, 37, 38 and 323 (preferably including at least SEQ ID NO:19 and 38), or
two, three, four, five, six, seven or all of: SEQ ID NO:9, 12, 19, 325, 33, 37, 38 and 323 (preferably including at least SEQ ID NO:19 and 38), or
two, three, four, five, six, seven or all of: SEQ ID NO:9, 12, 19, 32, 33, 328, 38 and 323 (preferably including at least SEQ ID NO:19 and 38), or
two, three, four, five, six, seven or all of: SEQ ID NO:9, 340, 19, 32, 33, 37, 38 and 323 (preferably including at least SEQ ID NO:19 and 38), or
two, three, four, five, six, seven, eight, nine or all of: SEQ ID NO:9, 12, 19, 22, 32, 33, 37, 38, 39 and 40 (preferably including at least SEQ ID NO:19 and 38), or
two, three, four, five, six, seven, eight, nine or all of: SEQ ID NO:326, 12, 19, 22, 32, 33, 37, 38, 39 and 40 (preferably including at least SEQ ID NO:19 and 38), or
two, three, four, five, six, seven, eight, nine or all of: SEQ ID NO:9, 12, 19, 22, 325, 33, 37, 38, 39 and 40 (preferably including at least SEQ ID NO:19 and 38), or
two, three, four, five, six, seven, eight, nine or all of: SEQ ID NO:9, 12, 19, 22, 32, 33, 328, 38, 39 and 40 (preferably including at least SEQ ID NO:19 and 38), or
two, three, four, five, six, seven, eight, nine or all of: SEQ ID NO:9, 340, 19, 22, 32, 33, 37, 38, 39 and 40 (preferably including at least SEQ ID NO:19 and 38), or
two, three, four, five, six, seven, eight, nine, ten or all of: SEQ ID NO:9, 12, 19, 22, 32, 33, 37, 38, 39, 40 and 323 (preferably including at least SEQ ID NO:19 and 38), or
two, three, four, five, six, seven, eight, nine, ten or all of: SEQ ID NO:326, 12, 19, 22, 32, 33, 37, 38, 39, 40 and 323 (preferably including at least SEQ ID NO:19 and 38), or
two, three, four, five, six, seven, eight, nine, ten or all of: SEQ ID NO:9, 12, 19, 22, 325, 33, 37, 38, 39, 40 and 323 (preferably including at least SEQ ID NO:19 and 38), or
two, three, four, five, six, seven, eight, nine, ten or all of: SEQ ID NO:9, 12, 19, 22, 32, 33, 328, 38, 39, 40 and 323 (preferably including at least SEQ ID NO:19 and 38), or
two, three, four, five, six, seven, eight, nine, ten or all of: SEQ ID NO:9, 340, 19, 22, 32, 33, 37, 38, 39, 40 and 323 (preferably including at least SEQ ID NO:19 and 38), or
two, three, four, five, six, seven, eight, nine, ten, eleven, twelve or all of: SEQ ID NO: 9, 10, 12, 15, 19, 22, 32, 33, 37, 38, 39, 40 and 323 (preferably including at least SEQ ID NO:19 and 38), or
two, three, four, five, six, seven, eight, nine, ten, eleven, twelve or all of: SEQ ID NO: 326, 327, 12, 330, 19, 22, 32, 33, 37, 38, 39, 40 and 323 (preferably including at least SEQ ID NO:19 and 38), or
two, three, four, five, six, seven, eight, nine, ten, eleven, twelve or all of: SEQ ID NO: 9, 10, 12, 331, 19, 22, 325, 33, 37, 38, 39, 40 and 323 (preferably including at least SEQ ID NO:19 and 38), or
two, three, four, five, six, seven, eight, nine, ten, eleven, twelve or all of: SEQ ID NO: 9, 10, 12, 331, 19, 22, 32, 33, 328, 38, 39, 40 and 323 (preferably including at least SEQ ID NO:19 and 38), or
two, three, four, five, six, seven, eight, nine, ten, eleven, twelve or all of: SEQ ID NO: 9, 10, 340, 15, 19, 22, 32, 33, 37, 38, 39, 40 and 323 (preferably including at least SEQ ID NO:19 and 38), or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 12, 15, 17, 19, 22, 24, 29, 30, 32, 33, 36, 37, 38, 39, 40, 44, 45 and 323 (preferably including at least SEQ ID NO:19 and 38), or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 340, 15, 342, 19, 22, 24, 343, 30, 32, 33, 36, 37, 38, 39, 40, 44, 45 and 323 (preferably including at least SEQ ID NO:19 and 38), or
two, three, four, five or all of SEQ ID NO:9, 19, 32, 33, 38 and 39 (preferably including at least SEQ ID NO:19 and 38).
two, three, four, five or all of: SEQ ID NO:326, 19, 32, 33, 38 and 39 (preferably including at least SEQ ID NO:19 and 38), or
two, three, four, five or all of: SEQ ID NO:9, 19, 325, 33, 38 and 39 (preferably including at least SEQ ID NO:19 and 38).

In another embodiment, the method comprises administration of 2, 3, 4, 5, 6, 7, 8, 9, 10 or all of the peptides selected from the group consisting of:
the immunogenic peptide defined in embodiment 1 (i),
the immunogenic peptide defined in embodiment 1 (ii),
the immunogenic peptide defined in embodiment 1 (iii),
the immunogenic peptide defined in embodiment 1 (iv),
the immunogenic peptide defined in embodiment 1 (v),
the immunogenic peptide defined in embodiment 1 (vii),
the immunogenic peptide defined in embodiment 1 (xiv),
the immunogenic peptide defined in embodiment 1 (xvi),
the immunogenic peptide defined in embodiment 1 (xxvi),
the immunogenic peptide defined in embodiment 1 (xxvii), and
the immunogenic peptide defined in embodiment 1 (xxviii).

In some embodiments, the method of the invention comprises administration of peptides derived from two or all three of: S protein, N protein and M protein. Using peptides based on multiple viral proteins can be an advantage to ensure protection against virus variants. For example, if a virus variant contains a mutation in the S protein, immune responses raised against the wild-type S protein might be less efficacious against the variant, but the responses raised against N or M based peptides would not be affected. Accordingly, in some embodiments, not all peptides are derived from the same protein, but the method comprises administration of peptides based on: the S protein and the N protein; the S protein and the M protein; the N protein and the M protein; or the S protein, the N protein and the S protein.

In some embodiments, the method of the invention comprises a modification to skew T cell responses towards antigen-specific interferon-gamma producing CD4+ and CD8+ T cells. In a preferred embodiment, said method elicits T cell immunity and/or an antibody response in said human subject.

In a further aspect, the invention relates to a method for the treatment, such as therapeutic treatment or prevention, of a SARS-CoV-related disease comprising administration to a human subject of one or more polynucleotides according the invention, wherein said polynucleotide(s) encode(s) the immunogenic peptides as defined herein above.

### Immunogenic Compositions

In a further main aspect, the invention relates to an immunogenic composition comprising one or more immunogenic peptides according to the invention or a polynucleotide according to the invention and a pharmaceutically-acceptable carrier. Pharmaceutically-acceptable carriers are well-known in the art.

Immunogenic compositions of the invention are preferably for, and therefore formulated to be suitable for, administration to a human subject. Preferably, the administration is parenteral, e.g. intravenous, subcutaneous, intramuscular, intradermal intracutaneous and/or intratumoral administration, i.e. by injection.

The immunogenic compositions are preferably chemically stable, i.e. the peptides in the composition do not chemically degrade or decompose. Thus, preferably, the amount of un-degraded, un-decomposed and/or unreacted peptides within the solution and/or composition is at least 90%, 91%, 92%, 93%, 94%, 95%, 96%, 97%, 98%, 99% or even 100% by weight as compared to its original, after storage of the solution or liquid composition for at least about 0.5, 1, 1.5, 2 or at least 3 hours at room temperature. Chemical stability can be assessed using any suitable technique known in the art, for instance using UPLC/MS as exemplified herein. When using UPLC/MS, a solution/composition is defined as chemically stable if the total % area of peaks that do not represent the desired peptide product in the UV spectrum after storage of at least about 0.5, 1, 1.5, 2 or at least 3 hours at room temperature is at most 20%, 15%, 10%, 9%, 8%, 7%, 6%, 5%, 4%, 3%, 2%, 1%, or 0% as compared to its original.

The immunogenic compositions are preferably also physically stable, i.e. the peptides in the composition do not precipitate or re-disperse. Physical stability can be assessed using any suitable technique known in the art, for instance by visual inspection or by particle distribution using a Malvern Mastersizer as exemplified herein, wherein average particle size is expressed in D(0.5). When using Malvern Mastersizer for assessing physical stability as exemplified herein, a solution/composition is defined as physically stable if the average D (0.5) after storage of at least about 0.5, 1, 1.5, 2 or at least 3 hours at room temperature is increased at most 50%, 40%, 30%, 20%, 10% or 5% as compared to its original (i.e. the freshly prepared solution directly after preparation). Preferably, a solution/composition is defined as physically stable if the average D(0.5) after storage of 3 hours at room temperature is increased at most 50%, 40%, 30%, 20%, 10% or 5%, preferably at most 20%, as compared to its original.

In one embodiment, the immunogenic composition comprises or consists of a mixture of dry or lyophilized peptides that are to be administered together.

### Vaccines

In a further main aspect, the invention relates to a vaccine (i.e. a vaccine product) comprising one or more immunogenic peptides of the invention or one or more immunogenic compositions of the invention.

In one embodiment, the vaccine product is a kit comprising two or more parts, e.g. two or more vials, wherein a plurality of immunogenic peptides of the invention are distributed over said two or more parts, e.g. distributed over two or more vials. For example, the peptides may be distributed over two or more immunogenic compositions. Embodiments of such immunogenic compositions are described herein below. In such an embodiment, the compositions may be mixed before administration of the vaccine to the patient or the compositions may be administered separately. In one embodiment, the vaccine comprises two or more compositions comprising dried or lyophilized peptides and the vaccine further comprises a reconstitution solution and optionally an adjuvant, wherein the adjuvant may be comprised within the reconstitution solution or be provided in a further separate vial.

In a preferred embodiment, the vaccine product comprises one or more immunogenic compositions, wherein the one or more immunogenic compositions comprise:
an immunogenic peptide as defined in embodiment 1 (xxx) and an immunogenic peptide as defined in embodiment 1 (xxii), or
an immunogenic peptide as defined in embodiment 1 (xxx) and an immunogenic peptide as defined in embodiment 1 (xxviii), or
an immunogenic peptide as defined in embodiment 1 (xxii) and an immunogenic peptide as defined in embodiment 1 (xxviii), or
an immunogenic peptide as defined in embodiment 2 (xxx) and an immunogenic peptide as defined in embodiment 2 (xxii), or
an immunogenic peptide as defined in embodiment 2 (xxx) and an immunogenic peptide as defined in embodiment 2 (xxviii), or
an immunogenic peptide as defined in embodiment 2 (xxii) and an immunogenic peptide as defined in embodiment 2 (xxviii), or
an immunogenic peptide as defined in embodiment 3 (xxx) and an immunogenic peptide as defined in embodiment 3 (xxii), or
an immunogenic peptide as defined in embodiment 3 (xxx) and an immunogenic peptide as defined in embodiment 3 (xxviii), or
an immunogenic peptide as defined in embodiment 3 (xxii) and an immunogenic peptide as defined in embodiment 3 (xxviii), or
an immunogenic peptide as defined in embodiment 1 (xxx), an immunogenic peptide as defined in embodiment 1 (xxii), and an immunogenic peptide as defined in embodiment 1 (xxviii), or
an immunogenic peptide as defined in embodiment 2 (xxx), an immunogenic peptide as defined in embodiment 2 (xxii), and an immunogenic peptide as defined in embodiment 2 (xxviii), or
an immunogenic peptide as defined in embodiment 3 (xxx), an immunogenic peptide as defined in embodiment 3 (xxii), and an immunogenic peptide as defined in embodiment 3 (xxviii), or
two, three, four, five, six or all seven of: the immunogenic peptides as defined in embodiment 1 (ii), embodiment 1 (xxxiii), embodiment 1 (xxx), embodiment 1 (ix), embodiment 1 (x), embodiment 1 (xxi) and embodiment 1 (xxii), or
two, three, four, five, six or all seven of: the immunogenic peptides as defined in embodiment 2 (ii), embodiment 2 (xxxiii), embodiment 2 (xxx), embodiment 2 (ix), embodiment 2 (x), embodiment 2 (xxi) and embodiment 2 (xxii), or
two, three, four, five, six or all seven of: the immunogenic peptides as defined in embodiment 3 (ii), embodiment 3 (xxxiii), embodiment 3 (xxx), embodiment 3 (ix), embodiment 3 (x), embodiment 3 (xxi) and embodiment 3 (xxii), or
two, three, four, five, six, seven or all eight of: the immunogenic peptides as defined in embodiment 1 (ii), embodiment 1 (xxxiii), embodiment 1 (xxx), embodiment 1 (ix), embodiment 1 (x), embodiment 1 (xxi), embodiment 1 (xxii) and embodiment 1 (xxviii), or
two, three, four, five, six, seven or all eight of: the immunogenic peptides as defined in embodiment 2 (ii), embodiment 2 (xxxiii), embodiment 2 (xxx), embodiment 2 (ix), embodiment 2 (x), embodiment 2 (xxi), embodiment 2 (xxii) and embodiment 2 (xxviii), or
two, three, four, five, six, seven or all eight of: the immunogenic peptides as defined in embodiment 3 (ii), embodiment 3 (xxxiii), embodiment 3 (xxx), embodiment 3 (ix), embodiment 3 (x), embodiment 3 (xxi), embodiment 3 (xxii) and embodiment 3 (xxviii).
   wherein the immunogenic peptides optionally are distributed over two or three immunogenic compositions.

In a further aspect, the invention relates to a vaccine product comprising one or more immunogenic compositions, wherein the one or more immunogenic compositions comprise the peptides set forth in:
SEQ ID NO:19 and SEQ ID NO:38, or
SEQ ID NO:19 and SEQ ID NO:323, or
SEQ ID NO:38 and SEQ ID NO:323, or
SEQ ID NO:19, SEQ ID NO:38 and SEQ ID NO:323, or
two, three or all of SEQ ID NO:19, 22, 38 and 323, or
two, three, four, five, six or all of: SEQ ID NO:9, 12, 19, 32, 33, 37 and 38 (preferably including at least SEQ ID NO:19 and 38), or
two, three, four, five, six or all of: SEQ ID NO:326, 12, 19, 32, 33, 37 and 38 (preferably including at least SEQ ID NO:19 and 38), or
two, three, four, five, six or all of: SEQ ID NO:9, 12, 19, 325, 33, 37 and 38 (preferably including at least SEQ ID NO:19 and 38), or
two, three, four, five, six or all of: SEQ ID NO:9, 12, 19, 32, 33, 328 and 38 (preferably including at least SEQ ID NO:19 and 38), or
two, three, four, five, six or all of: SEQ ID NO:9, 340, 19, 32, 33, 37 and 38 (preferably including at least SEQ ID NO:19 and 38), or
two, three, four, five, six, seven or all of: SEQ ID NO:9, 12, 19, 32, 33, 37, 38 and 323 (preferably including at least SEQ ID NO:19 and 38), or
two, three, four, five, six, seven or all of: SEQ ID NO:326, 12, 19, 32, 33, 37, 38 and 323 (preferably including at least SEQ ID NO:19 and 38), or
two, three, four, five, six, seven or all of: SEQ ID NO:9, 12, 19, 325, 33, 37, 38 and 323 (preferably including at least SEQ ID NO:19 and 38), or
two, three, four, five, six, seven or all of: SEQ ID NO:9, 12, 19, 32, 33, 328, 38 and 323 (preferably including at least SEQ ID NO:19 and 38), or
two, three, four, five, six, seven or all of: SEQ ID NO:9, 340, 19, 32, 33, 37, 38 and 323 (preferably including at least SEQ ID NO:19 and 38), or
two, three, four, five, six, seven, eight, nine or all of: SEQ ID NO:9, 12, 19, 22, 32, 33, 37, 38, 39 and 40 (preferably including at least SEQ ID NO:19 and 38), or
two, three, four, five, six, seven, eight, nine or all of: SEQ ID NO:326, 12, 19, 22, 32, 33, 37, 38, 39 and 40 (preferably including at least SEQ ID NO:19 and 38), or
two, three, four, five, six, seven, eight, nine or all of: SEQ ID NO:9, 12, 19, 22, 325, 33, 37, 38, 39 and 40 (preferably including at least SEQ ID NO:19 and 38), or
two, three, four, five, six, seven, eight, nine or all of: SEQ ID NO:9, 12, 19, 22, 32, 33, 328, 38, 39 and 40 (preferably including at least SEQ ID NO:19 and 38), or
two, three, four, five, six, seven, eight, nine or all of: SEQ ID NO:9, 340, 19, 22, 32, 33, 37, 38, 39 and 40 (preferably including at least SEQ ID NO:19 and 38), or
two, three, four, five, six, seven, eight, nine, ten or all of: SEQ ID NO:9, 12, 19, 22, 32, 33, 37, 38, 39, 40 and 323 (preferably including at least SEQ ID NO:19 and 38), or
two, three, four, five, six, seven, eight, nine, ten or all of: SEQ ID NO:326, 12, 19, 22, 32, 33, 37, 38, 39, 40 and 323 (preferably including at least SEQ ID NO:19 and 38), or
two, three, four, five, six, seven, eight, nine, ten or all of: SEQ ID NO:9, 12, 19, 22, 325, 33, 37, 38, 39, 40 and 323 (preferably including at least SEQ ID NO:19 and 38), or
two, three, four, five, six, seven, eight, nine, ten or all of: SEQ ID NO:9, 12, 19, 22, 32, 33, 328, 38, 39, 40 and 323 (preferably including at least SEQ ID NO:19 and 38), or
two, three, four, five, six, seven, eight, nine, ten or all of: SEQ ID NO:9, 340, 19, 22, 32, 33, 37, 38, 39, 40 and 323 (preferably including at least SEQ ID NO:19 and 38), or
two, three, four, five, six, seven, eight, nine, ten, eleven, twelve or all of: SEQ ID NO: 9, 10, 12, 15, 19, 22, 32, 33, 37, 38, 39, 40 and 323 (preferably including at least SEQ ID NO:19 and 38), or
two, three, four, five, six, seven, eight, nine, ten, eleven, twelve or all of: SEQ ID NO: 326, 327, 12, 330, 19, 22, 32, 33, 37, 38, 39, 40 and 323 (preferably including at least SEQ ID NO:19 and 38), or
two, three, four, five, six, seven, eight, nine, ten, eleven, twelve or all of: SEQ ID NO: 9, 10, 12, 331, 19, 22, 325, 33, 37, 38, 39, 40 and 323 (preferably including at least SEQ ID NO:19 and 38), or
two, three, four, five, six, seven, eight, nine, ten, eleven, twelve or all of: SEQ ID NO: 9, 10, 12, 331, 19, 22, 32, 33, 328, 38, 39, 40 and 323 (preferably including at least SEQ ID NO:19 and 38), or
two, three, four, five, six, seven, eight, nine, ten, eleven, twelve or all of: SEQ ID NO: 9, 10, 340, 15, 19, 22, 32, 33, 37, 38, 39, 40 and 323 (preferably including at least SEQ ID NO:19 and 38), or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 12, 15, 17, 19, 22, 24, 29, 30, 32, 33, 36, 37, 38, 39, 40, 44, 45 and 323 (preferably including at least SEQ ID NO:19 and 38), or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 340, 15, 342, 19, 22, 24, 343, 30, 32, 33, 36, 37, 38, 39, 40, 44, 45 and 323 (preferably including at least SEQ ID NO:19 and 38), or
two, three, four, five or all of SEQ ID NO:9, 19, 32, 33, 38 and 39 (preferably including at least SEQ ID NO:19 and 38).
two, three, four, five or all of: SEQ ID NO:326, 19, 32, 33, 38 and 39 (preferably including at least SEQ ID NO:19 and 38), or
two, three, four, five or all of: SEQ ID NO:9, 19, 325, 33, 38 and 39 (preferably including at least SEQ ID NO:19 and 38).
   wherein the immunogenic peptides optionally are distributed over two or three immunogenic compositions.

In a further aspect, the invention relates to a vaccine product comprising one or more immunogenic compositions, wherein the one or more immunogenic compositions comprise one or more polynucleotides of the invention, wherein said polynucleotide(s) encode(s) the immunogenic peptides as defined herein above.

SARS-CoV viruses continuously mutate and new variant strains start circulating in the human population. It is therefore envisaged that novel vaccines based on the sequences of the proteins of the variant virus may be required.

In a further aspect, the invention provides a method for adapting the immunogenic peptides, compositions and vaccine products to a new variant strain, based on a prediction as to whether replacing an original (reference) immunogenic peptide by a variant peptide would increase the total number of HLA ligands presented by a patient population wherein the variant virus is circulating (“variant background”).

Accordingly, in a further aspect, the invention relates to a method for selecting a peptide sequence for use in immunization against a SARS-CoV-related disease, comprising the steps of:

a) identify S, N and/or M variant protein sequences of a variant SARS-CoV strain circulating in the human population,
b) compare the reference peptide sequences according to the invention as described herein with the corresponding variant protein sequences, identify amino acid sequence differences and design a variant peptide sequence incorporating said amino acid difference(s),
c) analyze the effect of said amino acid sequence differences on predicted ligands by:
i) determining predicted ligand counts per HLA super-type for the reference peptide sequence in the variant background and for the variant peptide sequence in the variant background,
ii) assessing whether there is an increased total number of predicted ligands when using the variant peptide sequence in the variant background as compared to the number obtained with reference peptide sequence in the variant background,
   d) select the variant peptide sequence for use in immunization if an increased number of predicted ligands is obtained in step c) ii).

For step c) any suitable method for the prediction of ligands known in the art can be used. For example, ligands may be predicted using the MHCflurry method described by O'Donnell et al. (2018) Cell Systems 7, 129-132.

In a further step, the selected immunogenic peptide may be produced and optionally formulated into a composition for use in immunization (typically in combination with one or more of the other immunogenic peptides described herein).

Thus, in a further aspect, the invention relates to a method for producing an immunogenic peptide, said method comprising performing steps a), b), c) and d) as described here above and a further step of producing said variant peptide.

In one embodiment, said method comprises a further step of combining said variant peptide with one or more further immunogenic peptides as defined herein into a vaccine product or for combined use in immunization, wherein the one or more further immunogenic peptides do not include the reference peptide.

### Adjuvants

In one embodiment, an immunogenic composition or vaccine of the invention further comprises an adjuvant or the method of treatment further includes administration of an adjuvant.

The term “adjuvant” is used herein to refer to substances that have immune-potentiating effects and are co-administered, or added to, or co-formulated with an antigenic agent in order to enhance, induce, elicit, and/or modulate the immunological response against the antigenic agent when administered to a subject. In one embodiment, the adjuvant is physically linked, such as covalently linked, to the peptide(s) to be reconstituted.

In one embodiment, the adjuvant is an emulsifying adjuvant. In one embodiment, the adjuvant is an oil-based adjuvant. Oil-based adjuvants can be used to form emulsions (e.g. water-in-oil or oil-in-water emulsions) and are appreciated in the art to enhance and direct the immune response. Preferably the oil-based adjuvant is a mineral oil-based adjuvant. Non-limiting examples of oil-based adjuvants are bio-based oil adjuvants (based on vegetable oil/fish oil, etc.), squalene-based adjuvant (e.g. MF59), Syntex Adjuvant Formulation (SAF; Lidgate, Deborah M, Preparation of the Syntex Adjuvant Formulation (SAF, SAF-m, and SAF 1), In: Vaccine Adjuvants, Volume 42 of the series Methods in Molecular Medicine™ p 229-237, ISSN1543-1894), Freund's Complete Adjuvant (FCA), Freund's Incomplete Adjuvant (FIA), adjuvants based on peanut oil (e.g. Adjuvant 65), Lipovant (Byars, N. E., Allison, A. C., 1990. Immunologic adjuvants: general properties, advantages, and limitations. In: Zola, H. (Ed.), Laboratory Methods in Immunology. p 39-51), A504 (A. Tagliabue, R. Rappuoli Vaccine adjuvants: the dream becomes real Hum. Vaccine, 4 (5), 2008, p 347-349), Montanide adjuvants, which are based on purified squalene and squalene emulsified with highly purified mannide mono-oleate (e.g. Montanide ISA 25 VG, 28 VG, 35 VG, 50 V, 50 V2, 51 VG, 61 VG, 70 VG, 70 M VG, 71 VG, 720 VG, 760 VG, 763 A VG, 775 VG, 780 VG, 201 VG, 206 VG, 207 VG). More preferably, the oil-based adjuvant is Montanide ISA 51VG (Seppic), which is a mixture of Drakeol VR and mannide monooleate.

Other suitable adjuvants are adjuvants that are known to act via the Toll-like receptors and/or via a RIG-I (Retinoic acid-Inducible Gene-1) protein and/or via an endothelin receptor. Immune modifying compounds that are capable of activation of the innate immune system can be activated particularly well via Toll like receptors (TLRs), including TLRs 1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly(I:C). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLRS may be activated with bacterial flagellae or flagellin. TLR6 may be activated by mycobacterial lipoproteins and group B Streptococcus heat labile soluble factor (GBS-F) or Staphylococcus modulins. TLR7 may be activated by imidazoquinolines, such as imiquimod, resiquimod and derivatives imiquimod or resiquimod (e.g. 3M-052). TLR9 may be activated by unmethylated CpG DNA or chromatin-IgG complexes. Particularly preferred adjuvants comprise, but are not limited to, synthetically produced compounds comprising dsRNA, poly(I:C), poly I:CLC, unmethylated CpG DNA which trigger TLR3 and TLR9 receptors, IC31, a TLR 9 agonist, IMSAVAC, a TLR4 agonist, a water-in-oil emulsion comprising a mineral oil and a surfactant from the mannide monooleate family (e.g. Montanide ISA-51, Montanide ISA 720 an adjuvant produced by Seppic, France). RIG-I protein is known to be activated by ds-RNA just like TLR3 (Kato et al, (2005) Immunity, 1: 19-28).

A further particularly preferred TLR ligand is a Pam3cys and/or derivative thereof, preferably a Pam3cys lipopeptide or variant or derivative thereof, preferably such as described in WO2013051936A1 (incorporated herein by reference), more preferably U-Pam12 or U-Pam14 a.k.a. AMPLIVANT®.

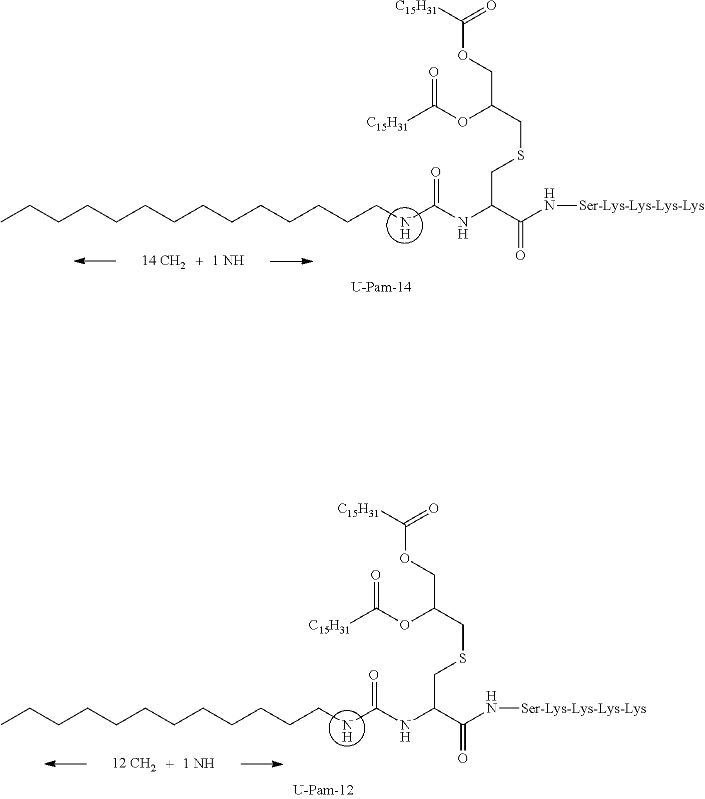

Pam3cys and/or derivatives thereof may optionally be covalently linked to the peptide antigen(s).

Further preferred adjuvants are Cyclic dinucleotides (CDNs), Muramyl dipeptide (MDP) and poly-ICLC. In a preferred embodiment, the adjuvants of the invention are non-naturally occurring adjuvants such as the Pam3cys lipopeptide derivative as described in WO2013051936A1, Poly-ICLC, imidazoquinoline such as imiquimod, resiquimod or derivatives thereof, CpG oligodeoxynucleotides (CpG-ODNs), such as class A-ODN (or K-type), class B-ODN (or D-type), class C-ODN as described in Sheiermann and Klinman, 2014 Vaccine 32(48): 6377-6389, more preferably class B-ODN (such as CpG7909 or 1018ISS) or class C-ODN (such as DV-281), having a non-naturally occurring sequence, and peptide-based adjuvants, such as muramyl dipeptide (MDP) or tetanus toxoid peptide, comprising non-naturally occurring amino acids.

Further preferred are adjuvants selected from the group consisting of: aluminum salts, Amplivax, AS 15, BCG, CP-870,893, CyaA, dSLIM, GM-CSF, IC30, IC31, ImuFact EV1P321, IS Patch, ISS, ISCOMATRIX, JuvImmune, LipoVac, MF59, monophosphoryl lipid A, Montanide IMS 1312, OK-432, OM-174, OM-197-MP-EC, ONTAK, PepTel®, vector system, PLGA microparticles, SRL172, Pam3Cys-GDPKHPKSF, YF-17D, VEGF trap, R848, beta-glucan, Aquila's QS21 stimulon, vadimezan, AsA404 (DMXAA), STING (stimulator of IFN genes) agonist (e.g. c-di-GMP VacciGrade™), PCI, NKT (natural killer T cell) agonist (e.g. alpha-galactosylceramide or alpha-GalCer, RNAdjuvant® (Curevac), retinoic acid inducible protein I ligands (e.g. 3pRNA or 5′-triphosphate RNA).

In a preferred embodiment, the method of the invention further comprises administration of an adjuvant, wherein said adjuvant preferably is:
a water-in-oil emulsion comprising a mineral oil and a surfactant from the mannide monooleate family, optionally combined with a TLR9 agonist, or
a TLR2 ligand.

### Dosages

Preferably, the vaccine or immunogenic composition of the invention comprises or consists of an amount of immunogenic peptides that constitutes a pharmaceutical dose. A pharmaceutical dose is defined herein as the amount of active ingredients (i.e. the total amounts of peptides in a peptide-based vaccine) that is applied to a subject at a given time point. A pharmaceutical dose may be applied to a subject in a single volume, i.e. a single shot, or may be applied in 2, 3, 4, 5 or more separate volumes that are applied preferably at different locations of the body, for instance in the right and the left limb. Reasons for applying a single pharmaceutical dose in separate volumes may be multiples, such as avoid negative side effects, avoiding antigenic competition and/or composition analytics considerations.

A pharmaceutical dose may be an effective amount or part of an effective amount. An “effective amount” is to be understood herein as an amount or dose of active ingredients required to prevent and/or reduce the symptoms of a disease relative to an untreated patient. The effective amount of active compound(s) used to practice the present invention for preventive and/or therapeutic treatment of a disease or condition varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective cellular T cell response or B cell response in the subject to be treated.

Preferably, pharmaceutical dose, or total amount of immunogenic peptides applied to a subject at a given time point, either in a single or in multiple injections at a certain time point, comprises an amount of peptides in the range from 0.1 μg to 20 mg, such as about 0.1 μg, 0.5 μg, 1 μg, 5 μg, 10 μg, 15 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 650 μg, 700 μg, 750 μg, 800 μg, 850 μg, 900 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg or about 20 mg or any value in between. Preferred ranges of pharmaceutical doses are from 0.1 μg to 20 mg, 1 μg to 10 mg, 10 μg to 5 mg, 0.5 mg to 2 mg, 0.5 mg to 10 mg or 1 mg to 5 mg or 2 to 4 mg.

In one embodiment, the vaccine or immunogenic composition of the invention is administered in a dose of between 1 μg and 300 μg, e.g. between 50 μg and 150 μg, such as approximately 100 μg of each peptide.

A single injection volume (i.e. volume applied on one location at a certain time point), comprising a total pharmaceutical dose, may between 100 μL and 2 mL, or between 100 μL and 1 mL. The single injection volume may be 100 μL, 200 μL, 300 μL, 400 μL, 500 μL, 600 μL, 700 μL, 800 μL, 900 μL, 1 mL, 1.1 mL, 1.2 mL, 1.3 mL, 1.4 mL, 1.5 mL, 1.6 mL, 1.7 mL, 1.8 mL, 1.9 mL, 2 mL, 3 mL or any value in between.

### Conditions to be Treated

Diseases to be treated therapeutically or prevented via the method of the invention or using the immunogenic peptides, composition or vaccines of the invention include all disorders or diseases that are related to an infection of a human subject with a SARS-CoV virus, including, but not limited to, SARS and COVID-19.

### Medical Treatment and Uses

As described above, the method of the invention involves treating a human subject with immunogenic peptides or immunogenic composition(s) or a vaccine according to the invention. If the immunogenic peptides to be used are divided over two or more compositions, these compositions may be mixed prior to administration and thus be co-administered, or they may be administered separately. Typically, all compositions, will be administered to the subject in a time interval of 24 hours, preferably within 4, 2 or 1 hour. If two or more compositions are administered, the administration may be at the same site, e.g. in the same limb, or at two or more different sites.

In the course of the treatment, the administration of the composition(s) may be carried out once or alternatively may be repeated (boosted) subsequently, such as, but not limited to, twice or three times. In one embodiment, a boost immunization is given after less than 28 days after the first immunization, such as less than 21 days, e.g. after between 5 and 20 days, such as after 7, 10 or 14 days. In one embodiment, the immunogenic composition used for the boost immunization has the same composition as the initially administered composition.

In another embodiment, the immunization is a single-dose vaccination, i.e. is not repeated within 6 months.

Preferably, the immunogenic compositions are administered in an effective amount as defined herein above. Preferably, administration is intravenous or subcutaneous, intracutaneous (intradermal) or intramuscular, optionally by injection or (slow) infusion. However, other administration routes can be envisaged, such as mucosal administration or respiratory inhalation.

The method of the invention may be part of a combination therapy, which may be provided as a separate treatment or added to the immunogenic composition of the invention. It is anticipated that antiviral therapies would have the greatest effect early in the course of disease, while immunosuppressive/anti-inflammatory therapies are likely to be more beneficial in the later stages of COVID-19. The method of the invention may e.g. be combined with drugs that affect viral replication (e.g. anti-viral compounds, neutralizing monoclonal antibody against the virus, chemotherapy, blood-derived immunoglobulin products, e.g. from convalescent plasma), modulate local or systemic immune responses (e.g. corticosteroids, kinase inhibitors, anti-chemokine therapies, interferons, anti-cytokine therapies or interleukin inhibitors such as antibodies against IL-6, IL-6R, TNF or IL-1), drugs that treat bacterial, viral or fungal co-infections (e.g. antibiotics, antifungals other antivirals), or anti-thrombotic therapy (e.g. anti-coagulant or anti-platelet therapy). An example for one of these combinations may be anti-IL-6 treatment to combat the cytokine release syndrome associated with acute respiratory distress (syndrome) or ARDS caused by SARS-CoV viruses in combination with the method of the invention, to stimulate virus-specific T and B cell responses at the same time, or sequentially. Another example for these combinations may be anti-SARS-CoV-2 antibody-based therapies to opsonize and eradicate virus particles, in the early stages of the disease, before the host has mounted an effective immune response against the infected cells, induced by the method of the invention. For patients requiring supplemental oxygen, combinations of the method of the invention with antiviral agents, like remdesivir, can be envisaged.

The method of the invention may be part of a combined prime-boost regimen, e.g. SLP treatment may be combined with a DNA vaccine format, RNA vaccine format, recombinant virus format or whole protein vaccine format based on (partial) sharing of SARS-CoV antigens. A combined prime-boost can e.g. be an initial vaccination with e.g. a DNA vaccine format (such as vaccination with a polynucleotide according to the invention or vaccination with plasmid DNA encoding a full-length SARS-CoV antigen) followed by one, two or more vaccinations with the immunogenic composition(s) of the invention or vice versa. In one embodiment, the boost immunization is given after less than 28 days after the first immunization, such as less than 21 days, e.g. after between 5 and 20 days, such as after 5, 7, 10 or 14 days.

In one embodiment, where the method is for the therapeutic treatment of a SARS-CoV-related disease, the subject to be treated is a subject who has been diagnosed to be infected with a SARS-CoV virus, such as SARS-CoV-2 or SARS-CoV-1. Thus, in such patients the objective is clearance of the infection and/or prevention of infection-related symptoms and/or treatment of an infection-related disease. In one embodiment, the method is for the therapeutic treatment of a SARS-CoV-related disease and a boost immunization is given after less than 28 days after the first immunization, such as less than 21 days, e.g. after between 5 and 20 days, such as after 5, 7, 10 or 14 days. In one embodiment, the immunogenic composition used for the boost immunization has the same composition as the initially administered composition.

In another embodiment, the invention provides a method of prevention of symptomatic infection by a SARS-CoV by administering to a human subject (or immunizing a human subject with) one or more immunogenic peptides with the aim to induce an immune response that prevents symptomatic infection in healthy human beings. In one embodiment, the method is for the prevention of symptomatic infection by a SARS-CoV and a boost immunization is given after less than 28 days after the first immunization, such as less than 21 days, e.g. after between 5 and 20 days, such as after 5, 7, 10 or 14 days. In one embodiment, the immunogenic composition used for the boost immunization has the same composition as the initially administered composition.

In another embodiment, the method is for the therapeutic treatment of a SARS-CoV-related disease, wherein the subject to be treated is a subject who has been previously been vaccinated with a prophylactic SARS-CoV vaccine, but who nevertheless has contracted a SARS-CoV infection, such as a SARS-CoV2 infection, for example due to an insufficient immune response to the prophylactic vaccine or due to an infection with a variant not or insufficiently recognized by a prophylactic SARS-CoV vaccine immune response, such as a SARS-CoV-2 vaccine immune response.

Thus, when method is for prevention, i.e. prophylactic treatment, of a SARS-CoV-related disease, the subject may be not-infected, but be a person at higher risk of getting infected with a SARS-CoV virus, such as medical staff or nursing home care staff or a person at higher risk of developing serious illness from infection with a SARS-CoV virus, such as an elderly person or a person with underlying medical problems, such as hypertension, cardiovascular disease, diabetes, chronic respiratory disease, obesity, auto-immune diseases, immunodeficiency syndromes or cancer. In such embodiments, the objective is prevention of infection, re-infection and/or disease for a certain minimum period of time, such as at least 3 months, such as at least 1 year, such as at least 3 years or lifelong immunity.

In another embodiment, the subject to be immunised is an individual belonging to higher risk category of people who may develop a severe form of the SARS-CoV2 induced pneumonia and/or complications, e.g.
a. people aged 65 years of age and older, with or without comorbidities;
b. adults of any age with the following clinical conditions:
   i. cancer;
   ii. chronic kidney disease or chronic renal failure;
   iii. Chronic Obstructive Pulmonary Disease (COPD);
   iv. cardiovascular or cerebrovascular conditions;
   v. chronic inflammation associated with autoimmune disease, heart disease, diabetes, cancer, arthritis, renal disease, and bowel diseases like Crohn's disease and ulcerative colitis;
   vi. immunocompromised patients, such as, but not limited to, cancer patients eligible to receive/on chemotherapy, autoimmune disease patients, HIV patients, transplant recipients;
   vii. obese people with BMI >30 kg/m2;
   viii. sickle cell disease patients;
   ix. heavy smokers;
   x. diabetes mellitus;
   xi. asthma;
   xii. cystic fibrosis;
   xiii. liver disease;
   xiv. fibrotic diseases of the lung(s), such as idiopathic pulmonary fibrosis;
   xv. interstitial lung disease;
   xvi. lung emphysema;
   xvii. chronic bronchitis;
   xviii. auto-immune conditions that require treatment with Disease Modifying Anti Rheumatic Drugs (DMARDS) and/or immunosuppressive agents.
c. adults of any age with immunodeficiencies or immunosuppression due to specific inherited or acquired diseases and/or due to treatment of these diseases, such as:
   i. patients with solid cancers treated with chemotherapy or irradiation or therapeutic antibodies/antibody drug conjugates (ADCs), e.g. causing durable immunosuppression;
   ii. patients with advanced or metastatic solid cancers due to disease-induced generalized immunosuppression by myeloid suppressor cells and other immunosuppressive mechanisms;
   iii. patients with hematologic malignancies with immunosuppression by the disease itself or by treatment with chemotherapy, including hematologic malignancy patients with B-cell-derived malignancies with e.g.:
      1. B-cell depleting monoclonal antibodies e.g. anti-CD20 antibody therapy, like rituximab;
      2. CAR T cells, targeting e.g. CD19, CD20 or CD22 or
      3. bi-specific antibodies, targeting e.g. CD19 and/or CD20;
   iv. Non-Hodgkin Lymphoma patients, such as Follicular Lymphoma or Diffuse large B-cell lymphoma (DLBCL) patients treated with anti-CD20 antibodies;
   v. patients with inherited or acquired immunodeficiencies, in particular B cell deficiencies, for example characterized by their reduced or absent capacity to produce IgG (neutralizing) antibodies against viruses in general and against SARS-CoV-2 in particular;
   vi. patients with auto-immune diseases treated with immunosuppressive medication, such as rheumatoid arthritis patients treated with B cell-depleting anti-CD20 antibody therapy (e.g. rituximab), with methotrexate or with anti-TNF;
   vii. patients with other autoimmune diseases treated with corticosteroids (prednisone, dexamethasone), cyclosporine or azathioprine.
   viii. patients with persistent HIV infections, especially those with low T cell numbers in the blood (<600/mm3)
   ix. patients with solid organ transplants in need of permanent treatment with immunosuppressive medication
   x. patients recovering from hemopoietic stem cell transplantation or bone marrow transplantation, particularly before they have recovered full immunocompetence

In one embodiment, the medical treatment is for therapeutic treatment or prevention of disease related to an infection with a virus of the Coronaviridae which has an S protein more than >65% sequence identity, such as more than 75% or more than 90% sequence identity with the S protein of SARS-CoV-2.

In one embodiment, the medical treatment is for therapeutic treatment or prevention of disease related to an infection with a virus of the Coronaviridae which has an N protein more than >75% sequence identity, such as more than 90% sequence identify with the N protein of SARS-CoV-2.

In one embodiment, the medical treatment is for therapeutic treatment or prevention of disease related to an infection with a virus of the Coronaviridae which has an M protein more than >75% sequence identity, such as more than 90% sequence identify with the M protein of SARS-CoV-2.

### Preparation of Immunogenic Compositions

Immunogenic compositions and vaccines of the invention may be prepared by any suitable method. In some embodiment, the immunogenic composition(s) are prepared from dried, preferably lyophilized, immunogenic peptides.

For example, the composition may be prepared by a method comprising the following steps:
a) providing a vial comprising dried, preferably lyophilized, peptides;
b) thawing the peptides, preferably for about 5-30 min;
c) adding a reconstitution composition to the vial comprising the peptides, preferably without swirling the vial;
d) allowing to admix, preferably for about 0.5-5 minutes; and
e) swirling until a clear solution is obtained, preferably for about 1-3 minutes.

Preferably, steps b) to e) are performed at room temperature.

Preferably, said vial comprises peptides in an amount for injection as a single volume in a method of treatment as defined herein, i.e. a single pharmaceutical dosage unit, or part thereof in case of multiple injections at difference locations of the subject's body at substantially the same time point.

In one embodiment, the reconstitution composition of step c) comprises or consists of DMSO and/or water-for-injection. In another embodiment, the reconstitution composition of step c) of the method for reconstituting peptides comprises or consists of about 60-80% v/v aqueous solution comprising an organic acid, about 5-10% v/v propylene glycol (CAS no. 57-55-6), about 10-20% v/v lower alcohol and about 5-10% v/v non-ionic hydrophilic surfactant. In one embodiment, the organic acid is citric acid and the citric acid is present in the aqueous solution in a concentration of about 0.05-0.1M. In one embodiment, the lower alcohol is ethanol. In one embodiment, the non-ionic hydrophilic surfactant:

a. is a mono-, di or triglyceride, preferably an ethoxylated triglyceride, and/or
b. has a hydrophilic-lipophilic balance (HLB) value between 9 and 14. In a further embodiment, the non-ionic hydrophilic surfactant is ethoxylated castor oil, preferably polyoxyethyleneglyceroltriricinoleate 35 (CAS no. 61791-12-6).

In one embodiment, the composition comprises or consists of about 75% v/v aqueous solution comprising about 0.1M citric acid, about 6.25% v/v propylene glycol (CAS no. 57-55-6), about 12.5% v/v ethanol and about 6.25% v/v polyoxyethyleneglyceroltriricinoleate 35 (CAS no. 61791-12-6).

Preferably, the amount of reconstitution composition in step c) is in a range of from about 0.5 and 2 mL, preferably 1 mL. Preferably, the amount of reconstituted peptides in step (a) is the total amount of reconstituted peptides as obtained after step e), i.e. within the clear solution obtained after step e).

In one embodiment, the reconstituted composition comprises or consists of about 1-2 mg/mL peptides, 0.038M citric acid, about 3.13% v/v propylene glycol (CAS no. 57-55-6), about 6.25% v/v ethanol, about 3.13% v/v polyoxyethyleneglyceroltriricinoleate 35 (CAS no. 61791-12-6) and about 50% of an oil-based adjuvant, preferably Montanide ISA 51 VG (Seppic), in water.

Dried peptides may be peptides free of further constituents but may also comprise buffer components such as trifluoroacetic acid (TFA), salts such as sodium, potassium or phosphate salts (e.g. NaCl, KCl and NaPO₄). The amount of further constituents is preferably less than 30%, more preferably less than 25%, of the total weight of the dry peptides to be reconstituted. Dried peptides to be reconstituted may be in a physical dried state as can be obtained by processes such as, but not limited to, rotor evaporation, lyophilization (freeze drying) and spray drying.

All patent and literature references cited in the present specification are hereby incorporated by reference in their entirety.

### EXAMPLES

### Example 1: SLP Design

A number of synthetic long peptides (SLPs) (Table 1) were designed by predicting the presence of HLA-I ligands in the SARS-CoV-2 S protein (SEQ ID NO:46), N protein (SEQ ID NO:47) and M protein (SEQ ID NO:48) sequences in silico by a machine learning algorithm. This prediction model had been trained on properties of 49119 unique data points. The properties were predictions of peptide processing stages by different tools, including predictions for: binding affinity to HLA molecules, binding affinity to the TAP protein complex binding affinity to HLA molecules, binding affinity to the TAP protein complex (Dönnes and Kohlbacher, Protein Sci. 2005 August; 14(8):2132-40) and proteasomal cleavage sites (Nielsen et al. (2005) Immunogenetics 57:33; Lazaro et al. (2015) Mol Immunol 68:72; Singh and Mishra (2016) Hum Immunol 77:295. To train the prediction model, a ligand training set was compiled from publicly available data in the Immune Epitope Database and Analysis Resource (www.iedb.org). The MHC ligand database was downloaded and filtered for 1) ligands detected by ligand elution and subsequent Mass Spectrometry, 2) ligands from human proteins and 3) length 8-11 amino acids. Sequences surrounding the identified ligands in the source proteins were used as false ligands. A logistic regression model was fit and performance was evaluated on an external ligand data set, compiled from MS of eluted ligands from a set of tumor samples from melanoma patients.

Additional criteria that were taken into account in SLP design included sequence conservation between SARS viruses, in particular SARS-CoV-2 and SARS-CoV-1 (S protein SEQ ID NO:49; N protein SEQ ID NO:50; M protein SEQ ID NO:51); SLP length with a preference for shorter SLPs for ease of manufacturing; presence of amino acids, ideally three amino acids, C-terminal of a predicted epitope to facilitate cleavage by the proteasome; and presence of epitopes described in literature, in particular Grifoni et al. (2020) Cell Host & Microbe 27:1-10.

Furthermore, ideal SLPs were selected by removing SLPs which are prone to degradation (i.e. loss of the original features of the purified peptide batch) based on their amino acid sequence. Peptides that contain one or more cysteines are susceptible to oxidation and may form disulfide bonds. Although the degree of oxidation during synthesis may be suppressed by introduction of thiol protecting groups, oxidation after the thiol deprotection step is difficult to control. Such features presumably affect the stability of synthesized peptide batches, rendering them less attractive for drug development. Therefore, SLPs with more than two cysteines or a cysteine at the N- or C-terminal or at both N- and C-terminals were not prioritized. Oxidation of the thioether moiety of methionines during manufacturing or storage may result into a sulfoxide or sulfone moiety. To minimalize this reaction, cleavage and deprotection was performed under an inert atmosphere and SLPs with more than three methionines were excluded of the ideal SLP sets. Lastly, peptides with an N-terminal glutamine were not prioritized for the ideal SLP sets as these SLPs are prone to pyroglutamate formation in the acidic aqueous media used during purification.

The following SLPs were designed:

TABLE 1A

| SLP # | | |
|---|---|---|
| (viral | | In silico predicted HLA-I |
| protein) | Sequence | ligands |
| 1 (S) | NAQALNTLVKQLSSNFGAISSV (SEQ ID NO: 1) | ALNTLVKQL |
| 2 (S) | LSSNFGAISSVLNDILSRLDKVEAE (SEQ ID | VLNDILSRL, SVLNDILSRL, |
| | NO: 2) | SVLNDILSR |
| 3 (S) | DKVEAEVQIDRLITGRLQSLQTY (SEQ ID NO: 3) | ITGRLQSLQTY, |
| | | RLITGRLQSLQ, LITGRLQSLQ, |
| | | GRLQSLQTY |
| 4 (S) | TQQLIRAAEIRASANLAATKMSE (SEQ ID NO: 4) | ASANLAATK, QQLIRAAEI |
| 5 (S) | KEIDRLNEVAKNLNESLIDLQELGK (SEQ ID | RLNEVAKNLNE, NLNESLIDL, |
| | NO: 5) | KEIDRLNEV, EVAKNLNESLI |
| 6 (S) | WPWYIWLGFIAGLIAIVMVT (SEQ ID NO: 6) | FIAGLIAIV, GLIAIVMV, |
| | | IWLGFIAGL, WPWYIWLGFIA |
| 7 (S) | DLQELGKYEQYIKWPWYIWLGFIAGLIAIVMVT | FIAGLIAIV, GLIAIVMV, |
| | (SEQ ID NO: 7) | QYIKWPWYI, QYIKWPWYIW, |
| | | KWPWYIWLGF, |
| | | QYIKWPWYIWL, KYEQYIKW, |
| | | KWPWYIWL, IWLGFIAGL, |
| | | YIKWPWYIW, |
| | | WPWYIWLGFIA, YEQYIKWPW |
| 8 (S) | ALQIPFAMQMAYRFNGIGVTQNVLYENQK (SEQ | LQIPFAMQM, |
| | ID NO: 8) | FAMQMAYRFNG, |
| | | QIPFAMQMAY, NGIGVTQNV, |
| | | IPFAMQMAY, LQIPFAMQMAY, |
| | | IPFAMQMAYRF |
| 9 (S) | ALNTLVKQLSSNFGAISSVLNDILSRLDKV (SEQ | VLNDILSRL, ALNTLVKQL, |
| | ID NO: 9) | SVLNDILSRL, SVLNDILSR |
| 10 (S) | VLNDILSRLDKVEAEVQIDRLITGRLQSLQTY | ITGRLQSLQTY, VLNDILSRL, |
| | (SEQ ID NO: 10) | RLITGRLQSLQ, RLDKVEAEV, |
| | | LITGRLQSLQ, GRLQSLQTY |
| 30 (S) | HSTQDLFLPFFSNVTWFHAIHVSGTN (SEQ ID | VTWFHAIHV, FLPFFSNVTWF, |
| | NO: 30) | LFLPFFSNVTW, |
| | | STQDLFLPFFS, |
| | | LPFFSNVTWFH, |
| | | NVTWFHAIHVS, |
| | | NVTWFHAIHV, LPFFSNVT, |
| | | FLPFFSNV |
| 31 (S) | GINITRFQTLLALHRSYLTPGDSSSG (SEQ ID | TLLALHRSYLT, LLALHRSYL, |
| | NO: 31) | NITRFQTLLAL, TLLALHRSYL, |
| | | INITRFQTLL, NITRFQTLLA |
| 32 (S) | PLVDLPIGINITRFQTLLALHRSYLTPGDSSSG | TLLALHRSYLT, LLALHRSYL, |
| | (SEQ ID NO: 32) | NITRFQTLLAL, TLLALHRSYL, |
| | | LPIGINITRFQ, INITRFQTLL, |
| | | NITRFQTLLA, LPIGINIT |
| 33 (S) | VEKGIYQTSNFRVQPTESIVRFPNITNL (SEQ ID | IYQTSNFRVQ, |
| | NO: 33) | KGIYQTSNFRV, |
| | | QPTESIVRFPN |
| 34 (S) | ASQSIIAYTMSLGAENSVAYSNNSI (SEQ ID | SIIAYTMSLG, SQSIIAYTMSL, |
| | NO: 34) | SVAYSNNSI, LGAENSVAYSN |
| 35 (S) | LDSKTQSLLIVNNATNVVIKVCEFQF (SEQ ID | IVNNATNVVIK, VIKVCEFQF, |
| | NO: 35) | NATNVVIKVCE, NVVIKVCEF |
| 21 (N) | KQRRPQGLPNNTASWFTALTQHGKE (SEQ ID | FTALTQHGK, NTASWFTALTQ, |
| | NO: 21) | GLPNNTASWFT, |
| | | LPNNTASWFT, LPNNTASW |
| 22 (N) | GANKDGIIWVATEGALNTPKDHIGTR (SEQ ID | IIWVATEGA, ALNTPKDHIG |
| | NO: 22) | |
| 23 (N) | TGPEAGLPYGANKDGIIWVATEGALNTPKDHIGTR | IIWVATEGA, ALNTPKDHIG |
| | (SEQ ID NO: 23) | |
| 24 (N) | VLQLPQGTTLPKGFYAEGSRGGSQASSRSSSRS | GTTLPKGFY, LQLPQGTTLPK, |
| | (SEQ ID NO: 24) | TTLPKGFYA |
| 25 (N) | VLQLPQGTTLPKGFYAEGSRGGSQASSRS (SEQ | GTTLPKGFY, LQLPQGTTLPK, |
| | ID NO: 25) | TTLPKGFYA |
| 26 (N) | GDAALALLLLDRLNQLESKMSGK (SEQ ID | LLLDRLNQL, ALALLLLDRLN, |
| | NO: 26) | LLLLDRLNQL, RLNQLESKMSG |
| 27 (N) | GMSRIGMEVTPSGTWLTYTGAIKL (SEQ ID | VTPSGTWLTYT, EVTPSGTWL, |
| | NO: 27) | GMSRIGMEVTP, TWLTYTGAI, |
| | | EVTPSGTWLT, TPSGTWLTY, |
| | | MEVTPSGTWL |
| 28 (N) | AFFGMSRIGMEVTPSGTWLTYT (SEQ ID NO: 28) | VTPSGTWLTYT, EVTPSGTWL, |
| | | GMSRIGMEVTP, |
| | | EVTPSGTWLT, TPSGTWLTY, |
| | | MEVTPSGTWL |
| 36 (N) | LPNNTASWFTALTQHGKEDLKFPRGQGVPINTN | FTALTQHGK, NTASWFTALTQ, |
| | (SEQ ID NO: 36) | FPRGQGVPI, LPNNTASWFT, |
| | | LPNNTASW |
| 37 (N) | SSPDDQIGYYRRATRRIRGGDGKMKDLS (SEQ | SSPDDQIG, YYRRATRRIRG, |
| | ID NO: 37) | IGYYRRATR, GYYRRATRRIR, |
| | | SPDDQIGYY |
| 38 (N) | KMKDLSPRWYFYYLGTGPEAGLPYGANK (SEQ | LSPRWYFYY, DLSPRWYFY, |
| | ID NO: 38) | DLSPRWYFYY, GTGPEAGL, |
| | | MKDLSPRWYFY, |
| | | KDLSPRWYFY, LSPRWYFYYL, |
| | | YLGTGPEA, KMKDLSPRW, |
| | | KDLSPRWYFYY, |
| | | KMKDLSPRWY, |
| | | KMKDLSPRWYF, |
| | | KDLSPRWYF, SPRWYFYYLGT |
| 39 (N) | GNGGDAALALLLLDRLNQLESKMSGKGQ (SEQ | LLLDRLNQL, ALALLLLDRLN, |
| | ID NO: 39) | LLLLDRLNQL, RLNQLESKMSG |
| 40 (N) | APSASAFFGMSRIGMEVTPSGTWLTYT (SEQ ID | VTPSGTWLTYT, EVTPSGTWL, |
| | NO: 40) | GMSRIGMEVTP, |
| | | EVTPSGTWLT, TPSGTWLTY, |
| | | MEVTPSGTWL |
| 41 (N) | PNFKDQVILLNKHIDAYKTFPPTEPKKDKKK (SEQ | LLNKHIDAY, KTFPPTEPKKD, |
| | ID NO: 41) | LLNKHIDAYK, KTFPPTEP, |
| | | KHIDAYKTF, VILLNKHIDAY, |
| | | ILLNKHID |
| 42 (N) | VILLNKHIDAYKTFPPTEPKKDKKK (SEQ ID | LLNKHIDAY, KTFPPTEPKKD, |
| | NO: 42) | LLNKHIDAYK, KTFPPTEP, |
| | | KHIDAYKTF, VILLNKHIDAY, |
| | | ILLNKHID |
| 43 (N) | NKHIDAYKTFPPTEPKKDKKK (SEQ ID NO: 43) | KTFPPTEPKKD, KTFPPTEP, |
| | | KHIDAYKTF |
| 44 (N) | YNVTQAFGRRGPEQTQGNFGDQ (SEQ ID | NVTQAFGRRGP |
| | NO: 44) | |
| 45 (N) | IRQGTDYKHWPQIAQFAPSASAFF (SEQ ID | QFAPSASA, KHWPQIAQFA, |
| | NO: 45) | RQGTDYKHW |
| 18 (M) | LWLLWPVTLACFVLAAVYRI (SEQ ID NO: 18) | TLACFVLA, WLLWPVTLA, |
| | | FVLAAVYRI, LWLLWPVTL, |
| | | LWPVTLACF, ACFVLAAVYRI, |
| | | WPVTLACFVL, LACFVLAA |
| 19 (M) | LFARTRSMWSFNPETNILLNV (SEQ ID NO: 19) | SMWSFNPETN, FNPETNILL, |
| | | LFARTRSMW, SFNPETNIL, |
| | | LFARTRSMWSF, |
| | | RTRSMWSFNP, |
| | | ARTRSMWSFNP, FARTRSMW, |
| | | FARTRSMWSF |
| 20 (M) | IKDLPKEITVATSRTLSYYKLGA (SEQ ID NO: 20) | TVATSRTLSY, VATSRTLSYY, |
| | | ATSRTLSYYKL, |
| | | ITVATSRTLSY, VATSRTLSY, |
| | | TVATSRTLSYY, ATSRTLSYYK, |
| | | ATSRTLSY, VATSRTLSYYK, |
| | | TSRTLSYYKLG, |
| | | SRTLSYYKLGA, ITVATSRTL, |
| | | LPKEITVATSR |
| 46 (N) | IRQGTDYKHWPQIAQFAPSASAFFG (SEQ ID | QFAPSASA, KHWPQIAQFA, |
| | NO: 323) | RQGTDYKHW |

Furthermore, on the basis of newly emerged variants of SARS-CoV2 circulating in the human population, the following SLPs were designed:

TABLE IB

| SLP # | | |
|---|---|---|
| (viral | | In silico predicted HLA-1 |
| protein) | Sequence | ligands (SEQ ID NO:) |
| 30-1 | HSTQDLFLPFFSNVTWFHAISGTN (SEQ ID | STQDLFLPFF (344), |
| | NO: 324 | FLPFFSNVTWF (345), |
| | | LFLPFFSNV (346), |
| | | PFFSNVTWFH (347), |
| | | FLPFFSNV (348), FFSNVTWF |
| | | (349), LFLPFFSNVTW (350), |
| | | STQDLFLPF (351), |
| | | TQDLFLPFF (352), |
| | | LPFFSNVTWF (353), |
| | | SNVTWFHAI (354), |
| | | FFSNVTWFHA (355), |
| | | LPFFSNVTWFH (356) |
| 32-1 | PLVDLPIGINITRFQTLHISYLTPGDSSSG (SEQ | FQTLHISYL (357), |
| | ID NO: 325) | RFQTLHISYLT (358), |
| | | RFQTLHISYL (359), |
| | | ITRFQTLHI (360), |
| | | INITRFQTLHI (361), |
| | | ITRFQTLHIS (362), |
| | | LPIGINITRF (363), |
| | | LPIGINITRFQ (364), |
| | | NITRFQTLHI (365), |
| | | GINITRFQ (366) |
| 9-1 | ALNTLVKQLSSNFGAISSVLNDILARLDKV (SEQ | VLNDILARL (367), |
| | ID NO: 326) | SVLNDILARL (368), |
| | | LVKQLSSNFGA (369), |
| | | SVLNDILAR (370) |
| 10-1 | VLNDILARLDKVEAEVQIDRLITGRLQSLQTY | ITGRLQSLQTY (371), |
| | (SEQ ID NO: 327) | RLDKVEAEV (372), |
| | | RLITGRLQSLQ (373), |
| | | RLQSLQTYV (374), |
| | | LITGRLQSLQ (375), |
| | | GRLQSLQTY (376) |
| 37-1 | SSRDDQIGYYRRATRRIRGGDGKMKDLS (SEQ | SSRDDQIG (377), |
| | ID NO: 328) | YYRRATRRIRG (378), |
| | | GYYRRATR (379), |
| | | SSRDDQIGY (380), |
| | | IGYYRRATR (381), |
| | | GYYRRATRRIR (382), |
| | | SSRDDQIGYYR (383) |
| 39-1 | GNGGDAALALLLLDRLNQLESKMFGKGQ (SEQ | LLLDRLNQL (384), |
| | ID NO: 329) | ALALLLLDRLN (385), |
| | | LLLLDRLNQL (386), |
| | | RLNQLESKMFG (387), |
| | | DAALALLLLDR (388) |

In addition, the following SLPs were designed with the aim of inducing B cell/antibody responses and potentially also T cell responses against the receptor-binding domain of the S protein.

A structural analysis of the receptor binding domain (RBD) of the S protein and the angiotensin converting enzyme 2 (ACE2) is reported by Yu Chen et al. (Biochem. Biophys. Res. Commun., https://doi.org/10.1016/j.bbrc.2020.02.071) and was used to identify amino acids residues that are part of RBD and ACE2 interaction in SARS-Coy. SARS-CoV-2 specific SLPs were designed based on the analogue amino acids in SARS-CoV-2 sequence that may have the same interaction. HLA II epitopes were predicted by NetMHCII 2.3 (Jensen et al., Immunology. 2018 July; 154(3):394-406) and NetMHCIIpan 3.2 (Jensen et al., Immunology. 2018 July; 154(3):394-406) HLA II ligand prediction tools. The combined output of these tools was used to determine potential epitopes:

TABLE 2A

| SLP # | | |
|---|---|---|
| (viral | | Predicted HLA-II and I |
| protein) | Sequence | ligands |
| 11 (S) | TGCVIAWNSNNLDSKVGGNYNYLYRLFR (SEQ | HLA-II: GCVIAWNSNNLDSKV |
| | ID NO: 11) | HLA-I: |
| | | KVGGNYNYLY, VIAWNSNNL, |
| | | KVGGNYNYL, NYNYLYRLF, |
| | | SKVGGNYNYLY, VGGNYNYLY, |
| | | NYNYLYRLFR, YNYLYRLFR |
| 12 (S) | SNNLDSKVGGNYNYLYRLFRKSNLK (SEQ ID | HLA-II: KVGGNYNYLYRLFRK, |
| | NO: 12) | VGGNYNYLYRLFRKS, |
| | | GGNYNYLYRLFRKSN, |
| | | GNYNYLYRLFRKSNL, |
| | | NYNYLYRLFRKSNLK |
| | | HLA-I: KVGGNYNYLY, |
| | | KVGGNYNYL, NYNYLYRLF, |
| | | SKVGGNYNYLY, VGGNYNYLY, |
| | | NYNYLYRLFR, YNYLYRLFR, |
| | | YLYRLFRKS |
| 13 (S) | TEIYQAGSTPCNGVEGFNCYFPL (SEQ ID | — |
| | NO: 13) | |
| 14 (S) | FNCYFPLQSYGFQPTNGVGYQPY (SEQ ID | HLA-I: CYFPLQSY, |
| | NO: 14) | YFPLQSYGFQP, |
| | | NCYFPLQSYGF, FPLQSYGFQP |
| 15 (S) | STPCNGVEGFNCYFPLQSYGFQPTNGVGY (SEQ | HLA-II: NGVEGFNCYFPLQSY, |
| | ID NO: 15) | GVEGFNCYFPLQSYG, |
| | | VEGFNCYFPLQSYGF, |
| | | EGFNCYFPLQSYGFQ, |
| | | GFNCYFPLQSYGFQP |
| | | HLA-I: CYFPLQSY, |
| | | YFPLQSYGFQP, |
| | | NCYFPLQSYGF, FPLQSYGFQP |
| 16 (S) | AGSTPCNGVEGFNCYFPLQSYGFQPTNGVGYQPY | HLA-II: NGVEGFNCYFPLQSY, |
| | (SEQ ID NO: 16) | GVEGFNCYFPLQSYG, |
| | | VEGFNCYFPLQSYGF, |
| | | EGFNCYFPLQSYGFQ, |
| | | GFNCYFPLQSYGFQP |
| | | HLA-I: CYFPLQSY, |
| | | YFPLQSYGFQP, |
| | | NCYFPLQSYGF, FPLQSYGFQP |
| 17 (S) | TGCVIAWNSNNLDSKVGGNYNYLYRLFRKSN | HLA-II: |
| | (SEQ ID NO: 17) | GCVIAWNSNNLDSKV, |
| | | KVGGNYNYLYRLFRK, |
| | | VGGNYNYLYRLFRKS, |
| | | GGNYNYLYRLFRKSN |
| | | HLA-I: KVGGNYNYLY, |
| | | VIAWNSNNL, KVGGNYNYL, |
| | | NYNYLYRLF, SKVGGNYNYLY, |
| | | VGGNYNYLY, NYNYLYRLFR, |
| | | YNYLYRLFR, YLYRLFRKS |
| 29 (S) | SKVGGNYNYLYRLFRKSNLKPFERDIS (SEQ ID | HLA-II: KVGGNYNYLYRLFRK, |
| | NO: 29) | VGGNYNYLYRLFRKS, |
| | | GGNYNYLYRLFRKSN, |
| | | GNYNYLYRLFRKSNL, |
| | | NYNYLYRLFRKSNLK, |
| | | YNYLYRLFRKSNLKP, |
| | | NYLYRLFRKSNLKPF, |
| | | YLYRLFRKSNLKPFE, |
| | | LYRLFRKSNLKPFER, |
| | | YRLFRKSNLKPFERD |
| | | HLA-I: KVGGNYNYLY, |
| | | KVGGNYNYL, RLFRKSNLKP, |
| | | NYNYLYRLF, SKVGGNYNYLY, |
| | | VGGNYNYLY, KSNLKPFER, |
| | | NYNYLYRLFR, YNYLYRLFR, |
| | | YLYRLFRKS, YRLFRKSNLKP |

Furthermore, on the basis of newly emerged variants of SARS-CoV2 circulating in the human population, the following SLPs were designed:

TABLE 2B

| SLP # | | |
|---|---|---|
| (viral | | In silico predicted HLA-I |
| protein) | Sequence | ligands (SEQ ID NO:) |
| 15-1 | STPCNGVEGFNCYFPLQSYGFQPTYGVGY (SEQ | LQSYGFQPTYG (389), |
| | ID NO: 330) | QSYGFQPTYG (390), |
| | | YGFQPTYGV (391), |
| | | CYFPLQSY (392), |
| | | YFPLQSYGFQP (393), |
| | | SYGFQPTY (394), |
| | | SYGFQPTYGV (395), |
| | | GVEGFNCYFP (396), |
| | | NCYFPLQSYGF (397), |
| | | FPLQSYGFQP (398) |
| 15-2 | STPCNGVKGFNCYFPLQSYGFQPTYGVGY (SEQ | LQSYGFQPTYG (399), |
| | ID NO: 331) | QSYGFQPTYG (400), |
| | | YGFQPTYGV (401), |
| | | KGFNCYFPL (402), |
| | | CYFPLQSY (403), |
| | | YFPLQSYGFQP (404), |
| | | SYGFQPTY (405), |
| | | SYGFQPTYGV (406), |
| | | GVKGFNCYFP (407), |
| | | NCYFPLQSYGF (408), |
| | | FPLQSYGFQP (409), |
| | | TPCNGVKGFNC (410) |
| 12-1 | SNNLDSKVGGNYNYRYRLFRKSNLK (SEQ ID | KVGGNYNYRY (411), |
| | NO: 340) | YRLFRKSN (412), RYRLFRKS |
| | | (413), NYNYRYRLF (414), |
| | | DSKVGGNYNYR (415), |
| | | SKVGGNYNYRY (416), |
| | | VGGNYNYRY (417), |
| | | KVGGNYNYR (418), |
| | | YNYRYRLFR (419), |
| | | NYNYRYRLFR (420), |
| | | GNYNYRYRL (421), |
| | | NYRYRLFRKSN (422) |
| 15-3 | STPCNGVQGFNCYFPLQSYGFQPTNGVGY (SEQ | CYFPLQSY (423), |
| | ID NO: 341) | YFPLQSYGFQP (424), |
| | | GVQGFNCYFP (425), |
| | | QGFNCYFPL (426), |
| | | NCYFPLQSYGF (427), |
| | | TPCNGVQGFNC (428), |
| | | FPLQSYGFQP (429) |
| 17-1 | TGCVIAWNSNNLDSKVGGNYNYRYRLFRKSN | KVGGNYNYRY (430), |
| | (SEQ ID NO: 342) | VIAWNSNNL (431), |
| | | NYNYRYRLF (432), |
| | | DSKVGGNYNYR (433), |
| | | SKVGGNYNYRY (434), |
| | | VGGNYNYRY (435), |
| | | KVGGNYNYR (436), |
| | | YNYRYRLFR (437), |
| | | NYNYRYRLFR (438), |
| | | GNYNYRYRL(439) |
| 29-1 | SKVGGNYNYRYRLFRKSNLKPFERDIS | KVGGNYNYRY (440), |
| | (SEQ ID NO: 343) | RLFRKSNLKP (441), |
| | | YRLFRKSN (442), RYRLFRKS |
| | | (443), NYNYRYRLF (444), |
| | | SKVGGNYNYRY (445), |
| | | VGGNYNYRY (446), |
| | | KVGGNYNYR (447), |
| | | YNYRYRLFR (448), |
| | | KSNLKPFER (449), |
| | | NYNYRYRLFR (450), |
| | | RLFRKSNLK (451), |
| | | GNYNYRYRL (452), |
| | | NYRYRLFRKSN (453), |
| | | YRLFRKSNLKP (454), |
| | | RLFRKSNL (455) |

### Example 2: In Vivo Studies in Mice—T Cell Responses

In wildtype C57BL/6 mice, prime-boost vaccinations were done with 6 or 20 SLPs. Pools of lyophilized SLP were dissolved in 10% DMSO/water for injection (WFI) at a concentration of 500 μg/SLP/mL and then emulsified with an equal amount of Montanide ISA 51 VG. 200 μl was used for vaccination per animal. Mice were vaccinated as indicated below, with 50 μg of each SLP.

Standard regimen: The 20 SLPs were divided in 4 non-overlapping pools of 5 SLP per group (see Table 3). Each mouse was vaccinated with 2 peptide pools (either pool 1 and 2 or pool 3 and 4); one in the left flank the other in the right flank. Vaccination was performed subcutaneously on day 0 and day 14. On day 21, mice were sacrificed, and serum and spleen were obtained.

TABLE 3
SLP # (SeqID #)

| Pool 1 | Pool 2 |
|---|---|
| SLP 30 (30) | SLP 32 (32) |
| SLP 12 (12) | SLP 15 (15) |
| SLP 10 (10) | SLP 36 (36) |
| SLP 38 (38) | SLP 24 (24) |
| SLP 44 (44) | SLP 40 (40) |
| Pool 3 | Pool 4 |
| SLP 33 (33) | SLP 17 (17) |
| SLP 29 (29) | SLP 9 (9) |
| SLP 5 (5) | SLP 37 (37) |
| SLP 22 (22) | SLP 39 (39) |
| SLP 45 (45) | SLP 19 (19) |

T cell responses: A single cell suspension was made from each spleen that was stimulated overnight in vitro with the individual SLPs. The next day, intracellular cytokine staining (ICS) was performed to detect IFNγ secreted by activated T cells in combination with staining for CD3, CD4 and CD8 surface markers by flow cytometry. The cells of interest were gated and depicted as percentage of their parent gate (i.e. CD4 T cells or CD8 T cells).

### Results T Cell Responses:

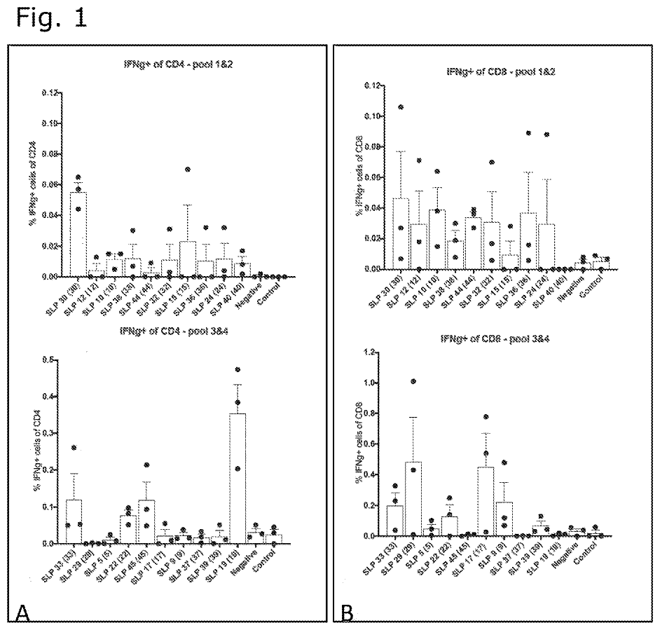

The immunization of wildtype C57BL/6 mice demonstrates that the majority of the designed SLPs to target an immune response against SARS-CoV-2 infected cells have biological activity in an in vivo model system. This was demonstrated by the capacity to elicit both CD4+ and CD8+ T cell responses after immunization. Both CD4⁺ and CD8⁺ T cells responses were observed in mice vaccinated with either pool 1&2 or with pool 3&4 (FIG. 1). The responses observed in these mice are higher than the background of the negative (non-stimulated) cells (Negative) and the cells stimulated with control SLP (Control). Some SLPs did not elicit CD4+ and CD8+ T cell responses. However, that does not necessarily mean that they are not immunogenic as MHC molecules capable of presenting ligands processed from the SLPs might not have been present in the mice.

### Example 3: In Vivo Studies in Mice—T Cell Responses

Six (6) of the twenty (20) SLPs that demonstrated a T cell response in Example 2 using C57BL/6 mice were used to determine the vaccination schedule limitations to induce meaningful immune responses in the shortest time frame.

TABLE 4

| | | | | | | | Day of |
|---|---|---|---|---|---|---|---|
| | | Dose | | | Dose of | | ICS |
| | Test | per | Dose of | Dose of | OX-40 | Days of | Read-out |
| Regimen | substance | SLP | Montanide | CpG | Ab | vaccination | (FACS) |
| Single | SLP in | 50 μg | 100 μL | 20 μg | — | 0 | 7 |
| shot | Montanide + | | | | | | |
| | CpG | | | | | | |
| Quick | SLP in | 50 μg | 100 μL | 20 μg | — | 0, 7 | 14 |
| | Montanide + | | | | | | |
| | CpG | | | | | | |
| Quick | SLP in | 50 μg | 100 μL | 20 μg | 150 μg | 0, 7 | 14 |
| | Montanide + | | | | | | |
| | CpG + OX- | | | | | | |
| | 40 Ab | | | | | | |
| Standard | SLP in | 50 μg | 100 μL | 20 μg | 150 μg | 0, 14 | 21 |
| | Montanide + | | | | | | |
| | CpG + OX- | | | | | | |
| | 40 Ab | | | | | | |

The selection of 6 SLPs were lyophilized as a pool. The lyophilized SLP pools were dissolved in 10% DMSO/water for injection (WFI) (10 μL DMSO and 90 μL WFI per mouse) and CpG was added (CpG ODN1826, 20 μg/mouse/injection) and then emulsified with an equal amount (100 μL) of Montanide ISA 51 VG. 200 μl was used for vaccination per animal. For the boost vaccination, OX-40 antibody (clone OX86, Panagioti et al, 2017) was subcutaneously injected in the neck in a volume of 200 μl to the indicated groups.

Standard regimen: Mice were vaccinated with 6 SLPs in one flank. Vaccination was performed subcutaneously on day 0 and day 14. On day 21, mice were sacrificed, and serum and spleen were obtained.

Quick regimen: Mice were vaccinated with 6 SLPs in one flank. Vaccination was performed subcutaneously on day 0 and day 7. On day 14, mice were sacrificed, and serum and spleen were obtained.

Single shot regimen: Mice were vaccinated with 6 SLPs in one flank. Vaccination was performed on day 0. On day 7, mice were sacrificed, and serum and spleen were obtained.

T cell responses: A single cell suspension was made from each spleen which was stimulated overnight in vitro with the individual SLPs. The next day, intracellular cytokine staining (ICS) was performed to detect IFNγ secreted by activated T cells in combination with staining for CD3, CD4 and CD8 surface markers by flow cytometry. The cells of interest were gated and depicted as percentage of their parent gate (i.e. CD4 T cells or CD8 T cells).

Results T Cell Responses:

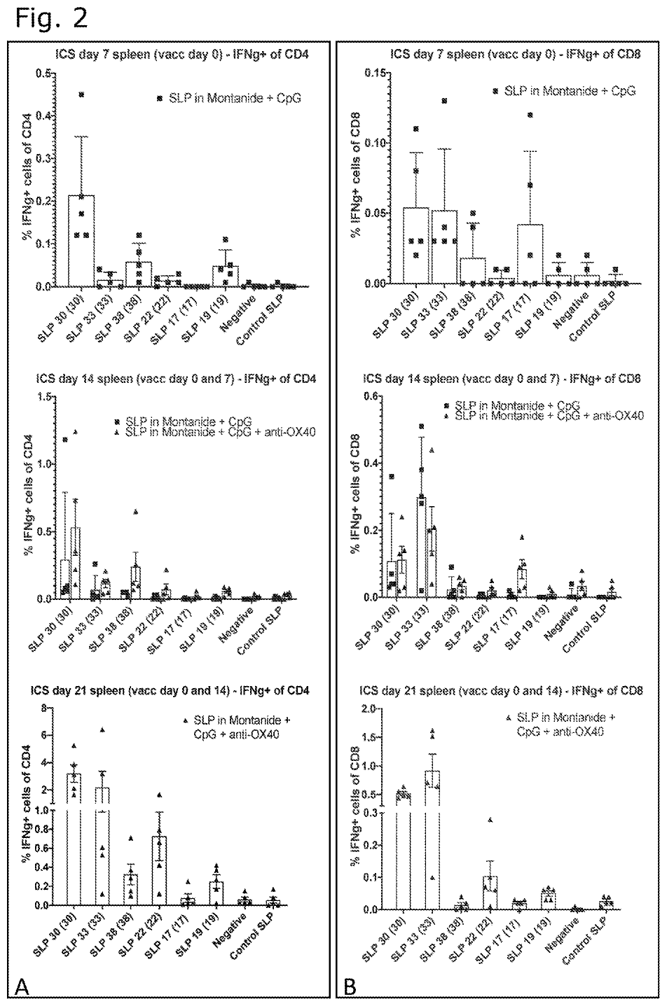

The immunization of wildtype C57BL/6 mice with the different regimens demonstrates that the designed SLPs are capable to elicit an antigen specific T cell response after one single vaccination with the SLPs. An alternative, quick regimen (boost at day 7 instead of day 14) demonstrates similar responses as compared to the standard regimen, supporting the capacity to elicit protective T cell immunity after one single vaccine injection and the option to adopt a flexible booster dosing scheme. This was demonstrated by the capacity to detect both CD4+ and CD8+ specific T cell responses amongst splenocytes, isolated 7 days after the last injection (FIG. 2).

### Example 4: In Vivo Studies in Mice—B Cell Responses

To assess the capacity of SLPs to elicit Spike protein binding antibodies, BALB/c mice, as a model to induce humoral responses, were vaccinated with the peptides of the invention.

Mice were vaccinated with 2 peptide pools. CpG ODN1826 was admixed with the resolved peptide pools and an agonistic OX-40 monoclonal antibody was co-injected to maximize the immune responses (as demonstrated in Panagioti et al 2017 Frontiers Immunol 8 and van Duikeren et al 2012 J Immunol 189). One pool was injected in the left flank and the other in the right flank. Vaccination was performed subcutaneously on day 0 and day 14. During the booster vaccination OX40 (clone OX86) was administered subcutaneous in the neck. On day 21, mice were sacrificed, and serum was obtained.

The serum obtained from mice was used to determine whether antibodies were able to recognize and bind to Spike protein (51 ectodomain and S2 ectodomain) using ELISA. Briefly, an ELISA was performed to determine presence of 51 and S2 extracellular domain-specific IgG antibodies in the serum of the vaccinated mice. Briefly, a NUNC MaxiSorp plate was coated overnight at 4 degrees with 100μl of the S1+S2ECD protein at a final concentration of 1 ug/mL in coating buffer. The next day, the plate was washed and blocked for 1 hour at room temperature with 200 uL blocking buffer (PBS/(1% BSA/0.05% Tween) per well. The plate was washed and 100 μl mouse serum was added in the indicated dilutions. Dilution was done in blocking buffer. This was incubated for 1 hour at room temperature. Thereafter the plate was washed and goat-anti-mouse IgG-HRP was added (1:4000 dilution, 100 μl/well). This was incubated for 1 hour at room temperature and thereafter washed. Next, the plates were developed by addition of 50 μL of TMB to each well and incubated at room temperature in the dark. 50 μL of stop solution (1M H2SO4) was added to stop the reaction. Optical density was measured within 5 minutes at 450 nm on20x in MQ diluted samples.

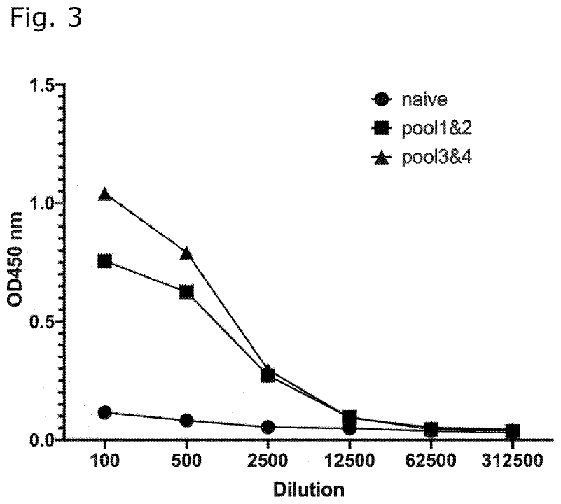

The results demonstrate that both pool combinations induce an antibody response against S. Addition of CpG (groups 3 and 5) greatly increases the level of IgG antibodies recognizing Spike protein (FIG. 3).

### Example 5: Ex Vivo Studies with Cells from Human Donors

### Induction of Primary T Cell Responses

Using cells from healthy donors obtained from the Sanquin Blood bank in the Netherlands, we studied whether it is possible to induce a primary T cell response in vitro with the designed SLPs. Cells from 2 healthy donors (HD I and HD II) were used for this study. Ten SLPs were selected based on the presence of predicted ligands for the HLA types of the donors. These 10 SLPs were divided in 2 pools. Pool 1 contained SLP 37, SLP 24, SLP 30, SLP 33 and SLP 5. Pool 2 contained: SLP 38, SLP 39, SLP 32, SLP 9 and SLP 19. Autologous monocyte-derived DCs were prepared and loaded overnight with a pool of 5 SLP (2 pools were tested). The next day these cells were used to stimulate autologous T cells. This was cultured for 10 days after which the PBMC were re-stimulated every week with freshly generated autologous monocyte-derived DCs loaded with SLPs. At the second and third re-stimulation, test 1 and test 2 were started, respectively. Here the T cell responses to the 10 individual SLPs were determined by IFNγ ELISpot, IFNγ ELISA and proliferation assay (³H-thymidine incorporation).

For testing, 0.25*10⁵ T cells were plated per well of a 96-well plate, in duplicate. Thereafter, 0.025*10⁵ autologous DCs loaded with individual SLPs were added. One plate was used for the IFNγ ELISpot analysis and one plate for the IFNγ ELISA and proliferation analyses. Plates were subsequently, incubated for 2 days at 37° C. After 2 days, 50μl culture supernatant was taken from each well for the IFNγ ELISA assay. Next, 50 μl of ³H-thymidine was added per well. After overnight incubation at 37° C., the plate was harvested and radioactive uptake was measured on a beta-counter to determine the extent of proliferation. For IFNγ ELISpot, the cells were transferred to a coated and blocked IFNγ ELISpot plate, after 2 days of incubation. ELISpot plates were incubated overnight at 37° C. and stained for IFNγ producing cells.

A summary of the results of this study are depicted in Table 5. An SLP was classified as capable to induce a primary T cell response when it had positive responses at the same test (i.e. test 1 or test 2) in the same donor detected with 2 different techniques or when testing positive at the two time points in the same donor with the same technique. Overall, positive primary T cell responses were detected to 6 of the 10 SLPs demonstrating that primary T cell responses can be induced to the designed SLPs. Some SLPs did not elicit responses. However, that does not necessarily mean that they are not immunogenic as HLA molecules capable of presenting ligands processed from the SLPs might not have been present in the donors.

TABLE 5
Detection of memory T cell responses in PBMC of
individuals recovered from SARS-CoV-2 infection.

| | | | | | | | positive response | |
|---|---|---|---|---|---|---|---|---|
| | | | | | | | detected on | |
| | | | | | | | one time | two time |
| | | | | | | | point with | points |
| | | | | | | | two | with the |
| | HD I: test 1 | | | HD I: test 2 | | | different | same |
| | ELISpot | ELISA | Proliferation | ELISpot | ELISA | Proliferation | assays | assay |
| SLP 37 | | | + | | | | | |
| SLP 24 | | | | | | | | |
| SLP 30 | | + | | | | | | |
| SLP 33 | | | + | | + | + | + | + |
| SLP 5 | | | | | | | | |
| SLP 38 | | | + | | | | | |
| SLP 39 | | | + | | | | | |
| SLP 32 | | | + | | | | | |
| SLP 9 | + | + | | | | | + | |
| SLP 19 | | | + | | | | | |
| | | | | | | | positive response | |
| | | | | | | | detected on | |
| | | | | | | | one time | two time |
| | | | | | | | point with | points |
| | | | | | | | two | with the |
| | HD II: test 1 | | | HD II: test 2 | | | different | same |
| | ELISpot | ELISA | Proliferation | ELISpot | ELISA | Proliferation | assays | assay |
| SLP 37 | | + | | | | | | |
| SLP 24 | | | | | | | | |
| SLP 30 | | | | | | | | |
| SLP 33 | | + | | | | | | |
| SLP 5 | | | | | | | | |
| SLP 38 | | + | | | + | | | + |
| SLP 39 | + | + | | | + | | + | + |
| SLP 32 | | + | | | + | | | + |
| SLP 9 | | | | | + | + | + | |
| SLP 19 | | + | | | + | + | + | + |

The memory T cell responses to 21 individual SLPs of the invention were tested in PMBCs that were obtained from 11 subjects that had cleared the SARS-CoV-2 virus (convalescent donors) and 4 healthy subjects (healthy donors). For this analysis a 4-day IFNγ ELISpot assay was used. In short, cryopreserved PBMC from healthy donors and SARS-CoV-2 convalescent donors were thawed. 2*10⁶ cells were incubated with each individual SLP in a concentration of 3 μM in a 24 wells plate or with 0.175 μg/mL of Candida antigen. When enough cells were available, SARS-CoV-2 S1, S, N and M peptivator mixes (Miltenyi Biotec B.V., Leiden, the Netherlands) were used as a control for the presence of a SARS-CoV-2 memory response. Two days later, Multiscreen plates were coated with an IFNγ coating antibody overnight at 4° C. The next day, the plates were washed 4× with PBS and blocking was done using IMDM 8% human serum (HS) at 37° C. for at least one hour. Cells were harvested, washed and counted. Around 100,000 cells were added per well of the coated Multiscreen plate and each sample was tested in triplicate. As a positive control, PHA (1 μg/mL) was added to cells that were not stimulated with SLP after thawing. The plate was cultured overnight at 37° C. thereafter, cells were discarded and the plate was washed using PBS/0.05% Tween-20. The IFNγ detection antibody (mAB7-B6-1-D1K, Mabtech) was diluted and added to each well and incubated for 2 hours at room temperature. Next, the plates were washed using PBS/0.05% Tween-20 and then incubated with streptavidin-ALP for 1 hour at room temperature. Each plate was washed with PBS/0.05% Tween-20. BCIP/NPT ALP substrate was filtered and added per well for 10-20 minutes at room temperature. Negative controls were non-stimulated PBMCs and PBMCs stimulated with a control (non-SARS-CoV-2 derived) SLP.

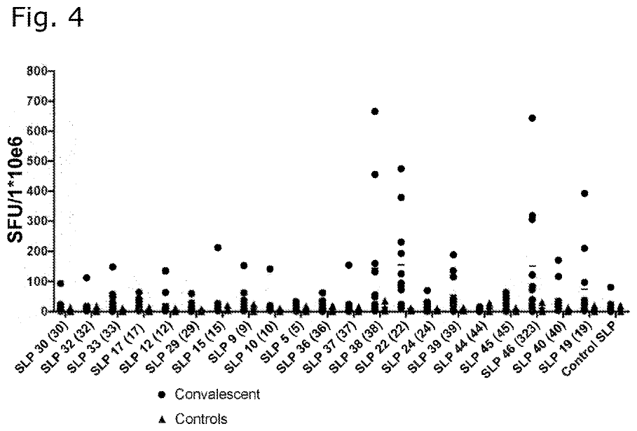

The detailed results of the outcome of this analysis are shown in FIG. 4 and Table 6A and B. The figure displays the spots per million PBMC while the table displays a stimulation index. Memory T cell responses were present in all presumed convalescent donor samples and absent in the healthy donor PBMC samples (as tested using Peptivator mixes, not shown). The IFNγ ELISpot results for the individual SLPs demonstrate that positive responses were detected to 13 of the 21 tested SLPs (FIG. 4), with a threshold set at 100 spots per 1,000,000 convalescent donor PMBC. Rendering the response to SLP 30 being just below the set threshold. In particular, SLP19, SLP22, SLP38 and SLP323 yielded high responses (FIG. 4). Responses to the SLPs in the healthy control samples remained below an average of 7.49 [ranging from 0.00-35.56] SFU/1*10e6 PMBC for all SLPs tested. Responses in the convalescent donor samples to the control SLP, based on an HBV antigen sequence, remained below an average of 15.08 [ranging from 0.00-82.22] SFU/1*10e6 PMBC.

The Stimulation Index (SI), calculated as relative responses of the SARS-CoV SLP versus the control SLP for each convalescent donor demonstrates that all SARS-CoV SLPs tested are positive in at least two donors, with a threshold set at SI>2.00 (indicated in bold in Table 6A and B). A positive response means that the SFU per 1*10e6 PBMC observed are at least 2 times higher compared to the SFU per 1*10e6 PBMC observed to the SLP control in that particular donor. Due to an SFU of zero to the control SLP in some donors, the SI could not be calculated for 2 convalescent donors and one healthy donor. The SI indexes for the control donor samples was negative for all SLPs tested (Table 6B)

TABLE 6A
Stimulation Index (SI) in convalescent donor samples

| | Convalescent Donors (numbered) | | | | | | | | | | |
|---|---|---|---|---|---|---|---|---|---|---|---|
| SLP ID (SeqID) | 1001 | 1002 | 1003 | 1004 | 1005 | 1006 | 1007 | 1008 | 1009 | 1010 | 1108 |
| SLP 30 (30) | 2.20 | N/A | 0.00 | 0.14 | 0.33 | 2.20 | 4.20 | 1.00 | N/A | 0.27 | 8.00 |
| SLP 32 (32) | 1.00 | N/A | 8.00 | 0.11 | 1.67 | 1.00 | 5.10 | 0.50 | N/A | 0.73 | 4.00 |
| SLP 33 (33) | 2.80 | N/A | 0.00 | 0.70 | 0.33 | 2.80 | 6.70 | 0.25 | N/A | 0.64 | 22.00 |
| SLP 17 (17) | 3.00 | N/A | 19.00 | 0.08 | 1.67 | 3.00 | 0.90 | 2.25 | N/A | 0.18 | 1.00 |
| SLP 12 (12) | 5.80 | N/A | 6.00 | 0.08 | 1.00 | 5.80 | 0.70 | 0.25 | N/A | 0.45 | 4.00 |
| SLP 29 (29) | 5.40 | N/A | 3.00 | 0.35 | 1.67 | 5.40 | 0.50 | 0.50 | N/A | 0.00 | 0.00 |
| SLP 15 (15) | 1.20 | N/A | 4.00 | 0.32 | 0.33 | 1.20 | 9.60 | 1.00 | N/A | 0.64 | 5.00 |
| SLP 9 (9) | 5.80 | N/A | 2.00 | 0.27 | 0.67 | 5.60 | 1.70 | 1.25 | N/A | 6.27 | 11.00 |
| SLP 10 (10) | 1.20 | N/A | 4.00 | 0.22 | 0.33 | 1.20 | 0.90 | 1.00 | N/A | 0.64 | 64.00 |
| SLP 5 (5) | 0.20 | N/A | 0.00 | 0.41 | 1.00 | 0.20 | 0.30 | 2.75 | N/A | 0.27 | 7.00 |
| SLP 36 (36) | 2.20 | N/A | 0.00 | 0.43 | 0.33 | 2.20 | 2.80 | 0.50 | N/A | 0.18 | 5.00 |
| SLP 37 (37) | 0.40 | N/A | 3.00 | 0.19 | 0.67 | 0.40 | 0.90 | 2.75 | N/A | 0.18 | 4.00 |
| SLP 38 (38) | 1.00 | N/A | 7.00 | 0.59 | 45.67 | 1.00 | 0.40 | 15.00 | N/A | 1.00 | 300.00 |
| SLP 22 (22) | 34.20 | N/A | 11.00 | 2.35 | 12.67 | 34.20 | 21.40 | 1.50 | N/A | 0.55 | 104.00 |
| SLP 24 (24) | 0.20 | N/A | 0.00 | 0.86 | 0.67 | 0.20 | 1.40 | 1.50 | N/A | 0.73 | 12.00 |
| SLP 39 (39) | 17.00 | N/A | 1.00 | 0.38 | 13.67 | 17.00 | 1.00 | 0.00 | N/A | 1.45 | 3.00 |
| SLP 44 (44) | 0.00 | N/A | 2.00 | 0.08 | 0.33 | 0.00 | 0.60 | 1.75 | N/A | 0.09 | 4.00 |
| SLP 45 (45) | 2.40 | N/A | 0.00 | 0.05 | 0.33 | 2.40 | 2.30 | 1.00 | N/A | 0.36 | 18.00 |
| SLP 46 (323) | 28.80 | N/A | 0.00 | 0.49 | 4.00 | 28.80 | 29.00 | 2.75 | N/A | 0.55 | 138.00 |
| SLP 40 (40) | 10.60 | N/A | 5.00 | 2.08 | 1.33 | 10.60 | 1.10 | 2.00 | N/A | 0.73 | 14.00 |
| SLP 19 (19) | 2.60 | N/A | 1.00 | 0.46 | 0.33 | 2.60 | 17.70 | 2.75 | N/A | 4.00 | 11.00 |
| Control SLP | 1.00 | (0 SFU) | 1.00 | 1.00 | 1.00 | 1.00 | 1.00 | 1.00 | (0 SFU) | 1.00 | 1.00 |
| (HBV antigen) | | | | | | | | | | | |

TABLE 6B
Stimulation Index (SI) in control donor samples

Healthy Donors (numbered)

| SLP ID (SeqID) | 2001 | 2002 | 2003 | 2004 |
|---|---|---|---|---|
| SLP 30 (30) | 1.33 | 0.00 | 0.56 | N/A |
| SLP 32 (32) | 0.67 | 0.33 | 0.89 | N/A |
| SLP 33 (33) | 0.33 | 0.00 | 0.56 | N/A |
| SLP 17 (17) | 0.00 | 0.00 | 0.67 | N/A |
| SLP 12 (12) | 0.00 | 0.67 | 0.56 | N/A |
| SLP 29 (29) | 0.33 | 0.00 | 0.44 | N/A |
| SLP 15 (15) | 2.00 | 0.67 | 1.00 | N/A |
| SLP 9 (9) | 0.00 | 0.00 | 1.22 | N/A |
| SLP 10 (10) | 0.00 | 0.00 | 0.56 | N/A |
| SLP 5 (5) | 0.00 | 0.00 | 0.89 | N/A |
| SLP 36 (36) | 0.00 | 0.33 | 1.00 | N/A |
| SLP 37 (37) | 0.00 | 0.67 | 0.78 | N/A |
| SLP 38 (38) | 1.33 | 0.67 | 1.78 | N/A |
| SLP 22 (22) | 0.33 | 1.00 | 0.56 | N/A |
| SLP 24 (24) | 1.00 | 0.33 | 0.44 | N/A |
| SLP 39 (39) | 0.33 | 0.67 | 0.67 | N/A |
| SLP 44 (44) | 0.00 | 0.33 | 0.89 | N/A |
| SLP 45 (45) | 0.00 | 0.00 | 0.56 | N/A |
| SLP 46 (323) | 1.00 | 0.00 | 1.56 | N/A |
| SLP 40 (40) | 0.33 | 0.00 | 0.67 | N/A |
| SLP 19 (19) | 0.00 | 0.00 | 1.00 | N/A |
| Control SLP | 1.00 | 1.00 | 1.00 | (0 SFU) |
| (HBV antigen) | | | | |

These data demonstrate that the positive SLPs contain T cell epitopes to which during a natural infection with SARS-CoV-2 an immune response was induced that was associated with clearance of the viral infection. In particular, SLP46 was remarkably immunogenic compared to the other SLPs.

Overall, the data demonstrate the immunogenic potential of the immunogenic peptides of the invention. Vaccines based on the peptides of the invention, which induce both CD4+ and CD8+ T cell responses, are expected to increase the duration of immunity compared to the relatively short-lived duration of prophylactic vaccines, which predominantly elicit protective neutralizing antibody immunity, often with low underlying T helper cell responses only directed against the Spike protein.

SEQ ID NO: 46
SARS-CoV-2 S protein / >sp|P0DTC2|SPIKE_SARS2 Spike glycoprotein
OS = Severe acute respiratory syndrome coronavirus 2 OX = 2697049 GN = S PE = 1 SV = 1
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
SEQ ID NO: 47
SARS-CoV-2 N protein / >sp|P0DTC9|NCAP_SARS2 Nucleoprotein
OS = Severe acute respiratory syndrome coronavirus 2 OX = 2697049 GN = N PE = 1 SV = 1
MSDNGPQNQRNAPRITFGGPSDSTGSNQNGERSGARSKQRRPQGLPNNTASWFTALTQHG
KEDLKFPRGQGVPINTNSSPDDQIGYYRRATRRIRGGDGKMKDLSPRWYFYYLGTGPEAG
LPYGANKDGIIWVATEGALNTPKDHIGTRNPANNAAIVLQLPQGTTLPKGFYAEGSRGGS
QASSRSSSRSRNSSRNSTPGSSRGTSPARMAGNGGDAALALLLLDRLNQLESKMSGKGQQ
QQGQTVTKKSAAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQVILLNKHIDAY
KTFPPTEPKKDKKKKADETQALPQRQKKQQTVTLLPAADLDDFSKQLQQSMSSADSTQA
SEQ ID NO: 48
SARS-CoV-2 M protein / >sp|P0DTC5|VME1_SARS2 Membrane protein
OS = Severe acute respiratory syndrome coronavirus 2 OX = 2697049 PE = 3 SV = 1
MADSNGTITVEELKKLLEQWNLVIGFLFLTWICLLQFAYANRNRFLYIIKLIFLWLLWPV
TLACFVLAAVYRINWITGGIAIAMACLVGLMWLSYFIASFRLFARTRSMWSFNPETNILL
NVPLHGTILTRPLLESELVIGAVILRGHLRIAGHHLGRCDIKDLPKEITVATSRTLSYYK
LGASQRVAGDSGFAAYSRYRIGNYKLNTDHSSSSDNIALLVQ
SEQ ID NO: 49
SARS-CoV-1 S protein / >sp|P59594|SPIKE_CVHSA Spike glycoprotein
OS = Human SARS coronavirus OX = 694009 GN = S PE = 1 SV = 1
MFIFLLFLTLTSGSDLDRCTTFDDVQAPNYTQHTSSMRGVYYPDEIFRSDTLYLTQDLFL
PFYSNVTGFHTINHTFGNPVIPFKDGIYFAATEKSNVVRGWVFGSTMNNKSQSVIIINNS
TNVVIRACNFELCDNPFFAVSKPMGTQTHTMIFDNAFNCTFEYISDAFSLDVSEKSGNFK
HLREFVFKNKDGFLYVYKGYQPIDVVRDLPSGFNTLKPIFKLPLGINITNFRAILTAFSP
AQDIWGTSAAAYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVADYSVLYNSTF
FSTFKCYGVSATKLNDLCFSNVYADSFVVKGDDVRQIAPGQTGVIADYNYKLPDDFMGCV
LAWNTRNIDATSTGNYNYKYRYLRHGKLRPFERDISNVPFSPDGKPCTPPALNCYWPLND
YGFYTTTGIGYQPYRVVVLSFELLNAPATVCGPKLSTDLIKNQCVNFNFNGLTGTGVLTP
SSKRFQPFQQFGRDVSDFTDSVRDPKTSEILDISPCSFGGVSVITPGTNASSEVAVLYQD
VNCTDVSTAIHADQLTPAWRIYSTGNNVFQTQAGCLIGAEHVDTSYECDIPIGAGICASY
HTVSLLRSTSQKSIVAYTMSLGADSSIAYSNNTIAIPTNFSISITTEVMPVSMAKTSVDC
NMYICGDSTECANLLLQYGSFCTQLNRALSGIAAEQDRNTREVFAQVKQMYKTPTLKYFG
GFNFSQILPDPLKPTKRSFIEDLLFNKVTLADAGFMKQYGECLGDINARDLICAQKFNGL
TVLPPLLTDDMIAAYTAALVSGTATAGWTFGAGAALQIPFAMQMAYRFNGIGVTQNVLYE
NQKQLANQHNKAISQIQESLTTTSTALGKLQDVVNQNAQALNTLVKQLSSNFGAISSVLN
DILSRLDKVEAEVQIDRLITGRLQSLQTYVTQQLIRAAEIRASANLAATKMSECVLGQSK
RVDFCGKGYHLMSFPQAAPHGVVFLHVTYVPSQERNFTTAPAICHEGKAYFPREGVFVFN
GTSWFITQRNFFSPQIITTDNTFVSGNCDVVIGIINNTVYDPLQPELDSFKEELDKYFKN
HTSPDVDLGDISGINASVVNIQKEIDRLNEVAKNLNESLIDLQELGKYEQYIKWPWYVWL
GFIAGLIAIVMVTILLCCMTSCCSCLKGACSCGSCCKFDEDDSEPVLKGVKLHYT
SEQ ID NO: 50
SARS-CoV-1 N protein / >sp|P59595/NCAP_CVHSA Nucleoprotein
OS = Human SARS coronavirus OX = 694009 GN = N PE = 1 SV = 1
MSDNGPQSNQRSAPRITFGGPTDSTDNNQNGGRNGARPKQRRPQGLPNNTASWFTALTQH
GKEELRFPRGQGVPINTNSGPDDQIGYYRRATRRVRGGDGKMKELSPRWYFYYLGTGPEA
SLPYGANKEGIVWVATEGALNTPKDHIGTRNPNNNAATVLQLPQGTTLPKGFYAEGSRGG
SQASSRSSSRSRGNSRNSTPGSSRGNSPARMASGGGETALALLLLDRLNQLESKVSGKGQ
QQQGQTVTKKSAAEASKKPRQKRTATKQYNVTQAFGRRGPEQTQGNFGDQDLIRQGTDYK
HWPQIAQFAPSASAFFGMSRIGMEVTPSGTWLTYHGAIKLDDKDPQFKDNVILLNKHIDA
YKTFPPTEPKKDKKKKTDEAQPLPQRQKKQPTVTLLPAADMDDFSRQLQNSMSGASADST
QA
SEQ ID NO: 51
SARS-CoV-1 M protein / >sp|P59596|VMEl_CVHSA Membrane protein
OS = Human SARS coronavirus OX = 694009 GN = M PE = 1 SV = 1
MADNGTITVEELKQLLEQWNLVIGFLFLAWIMLLQFAYSNRNRFLYIIKLVFLWLLWPVT
LACFVLAAVYRINWVTGGIAIAMACIVGLMWLSYFVASFRLFARTRSMWSFNPETNILLN
VPLRGTIVTRPLMESELVIGAVIIRGHLRMAGHSLGRCDIKDLPKEITVATSRTLSYYKL
GASQRVGTDSGFAAYNRYRIGNYKLNTDHAGSNDNIALLVQ

### Example 6: Design and Production of Variant Vaccines

Upcoming SARS-CoV-2 variants with the potential to become prevalent were identified through literature study and monitoring online resources such as NextStrain (https://nextstrain.org/ncov/global), PANGOLIN Coy-lineages (https://cov-lineages.org/), outbreak. info (https://outbreak.info/) and Virological (https://virological.org/). Currently variant vaccines are designed for lineages B.1.1.7, B.1.351 and P.1, in this example referred to as the UK, SA and BR variants. Lineage B.1.1.7, known as the UK variant, is an example of a SARS-CoV-2 variant that rapidly became prevalent in large parts of Europe. Lineage defining mutations for this variant and its parent lineages were collected (https://virological.org/t/563, https://virological.org/t/592, https://virological.org/t/605, https://cov-lineages.org/global_report_B.1.1.7.html, Ferrareze et al. 2021 https://doi.org/10.1101/2021.01.27.426895) and applied to the corresponding reference protein sequences of S protein (SEQ ID NO:46), N protein (SEQ ID NO:47) and M protein (SEQ ID NO:48), taking positional shifts that occur when introducing insertions or deletions into account. Similarly, lineage defining mutations for the SA, BR and IN variants and their parent lineages were collected. This analysis resulted in the variant N and S protein sequences set forth in SEQ ID NO:332-339.

Twenty-one reference SLP sequences from this invention (those listed in Table 6) were checked for overlap with the regions containing mutations and alternative variant SLP sequences containing the sequence differences were generated, creating variant-specific SLP sequences for SLP9, SLP10, SLP12, SLP15, SLP17, SLP29, SLP30, SLP-32, SLP-37 and SLP39 named SLP9-1, SLP10-1, SLP12-1, SLP15-1, SLP-15-2, SLP15-3, SLP17-1, SLP29-1, SLP30-1, SLP-32-1, SLP-37-1 and SLP39-1 (see Table 1B and 2B).

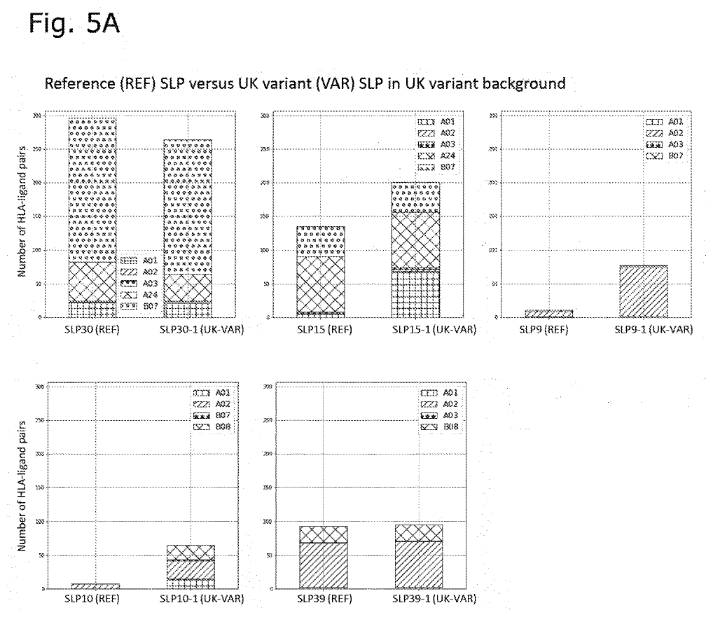

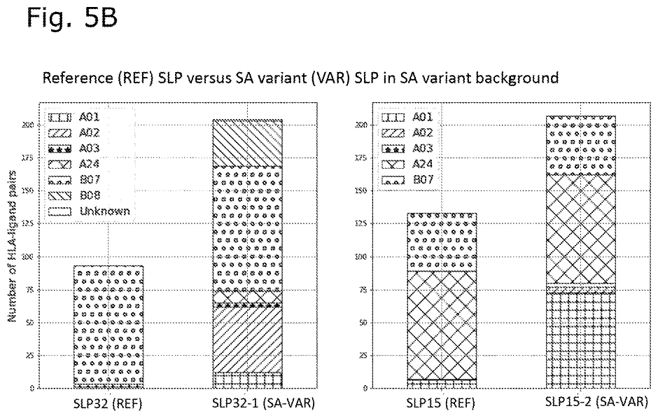

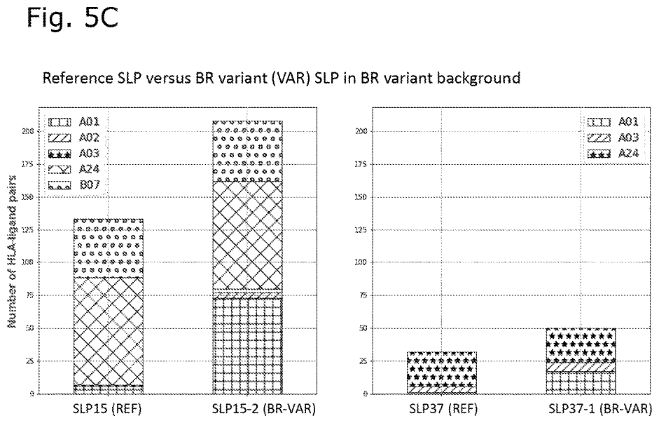

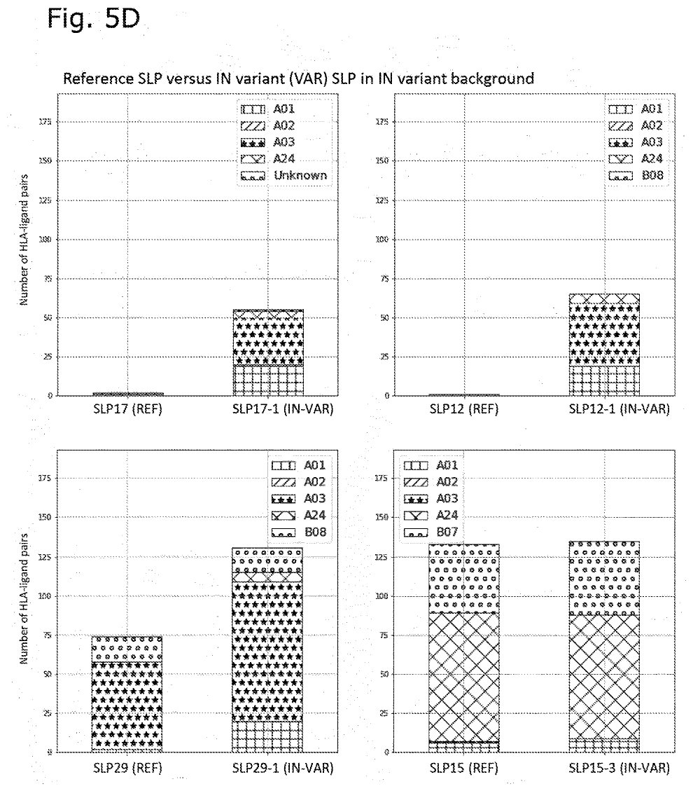

To quantify the effect of the mutations on the change in predicted immunogenicity, the full-length variant-specific protein sequences were analyzed with a range of algorithms that predict the likelihood that a ligand sequence will pass the various stages of antigen processing. The results of these tools were analyzed by a machine learning model to determine whether a ligand will be processed correctly and bind to an HLA molecule (for a more in-depth explanation, see “Example 1: SLP design” in this document). The predicted ligand content of each specific reference SLP and its variant counterpart were extracted from the resulting full-length variant-specific protein ligand dataset through mapping positions of the SLP (minus three C-terminal amino acids to account for a C-terminal proteasomal cleavage site). The HLA allele of each HLA-ligand combination is mapped back to its HLA supertype (A01, A02, A03, A24, B07 and B08 count) for both the variant dataset (i.e. SLP variant in variant background) and the reference dataset (i.e. the reference SLP in variant background), using the data of supplementary table 1 from Sidney et al. (BMC Immunol 9, 1 (2008)) to perform the supertype mapping. Lastly, an intersection is made with the ligand content from the reference SLP and its variant-specific counterpart in the variant background. The predicted ligand contents and the intersection counts for the set of 21 reference SLPs in three different variant settings (UK, SA, BR and IN) are provided for in Table 7A-D and depicted in FIG. 5A-D. These results demonstrate whether there is an increased total number of predicted ligands when using the variant peptide sequence in the variant background as compared to the number obtained with reference peptide sequence in the variant background. Such method assesses to which extent the number of ligands for the HLA supertypes is affected when a reference SLP is applied to a variant background (i.e. when an individual infected with the variant strain would be immunized with the reference SLP). For example, the difference in ligand count between SLP39 and SLP39-1 is one ligand and the effect on the HLA supertype distribution is therefore negligible. In this case, replacing the reference SLP for the variant-specific SLP for immunization of a patient infected with the variant virus would not be significantly beneficial. Based on the clear differences in ligand count, the alternatives for SLP9, SLP10, SLP12, SLP15, SLP17, SLP29, SLP32, SLP37: SLP9-1, SLP10-1, SLP12-1, SLP15-1, SLP-15-2, SLP17-1, SLP29-1, SLP32-1 and SLP-37-1 resp. would be beneficial when used in a certain immunogenic composition of the invention that aims to prevent or treat infection with a particular circulating variant strain.

In the above example, an HLA ligand prediction method that includes antigen processing was briefly described. Other, open-source methods to perform this type of analysis that include antigen processing exist. One notable example is MHCflurry (O'Donnell et al., Cell Systems, Oct. 21, 2020, https://github.com/openvax/mhcflurry), which combines all of these steps in one software package.

TABLE 7A
predicted ligand counts in SARX-CoV2 B.1.1.7 lineage (UK variant) background

| SLP | analysis setting | A01_count | A02_count | A03_count | A24_count | B07_count | B08_count |
|---|---|---|---|---|---|---|---|
| SLP 30 | SLP 30-1 in UK variant | 21 | 1 | 2 | 40 | 200 | 0 |
| | SLP 30 in UK variant | 22 | 1 | 1 | 58 | 214 | 0 |
| | intersection | −1 | 0 | +1 | −18 | −14 | 0 |
| SLP 15 | SLP 15-1 in UK variant | 68 | 3 | 3 | 82 | 45 | 0 |
| | SLP 15 in UK variant | 6 | 0 | 2 | 82 | 45 | 0 |
| | intersection | +62 | +3 | +1 | 0 | 0 | 0 |
| SLP 9 | SLP 9-1 in UK variant | 2 | 72 | 2 | 0 | 1 | 0 |
| | SLP 9 in UK variant | 1 | 8 | 0 | 0 | 2 | 0 |
| | intersection | +1 | +64 | +2 | 0 | −1 | 0 |
| SLP 10 | SLP 10-1 in UK variant | 15 | 27 | 0 | 0 | 1 | 22 |
| | SLP 10 in UK variant | 0 | 7 | 0 | 0 | 0 | 0 |
| | intersection | +15 | +20 | 0 | 0 | +1 | +22 |
| SLP 39 | SLP 39 in UK variant | 3 | 67 | 1 | 0 | 0 | 24 |
| | SLP 39 in UK variant | 2 | 66 | 1 | 0 | 0 | 24 |
| | intersection | +1 | +1 | 0 | 0 | 0 | 0 |

TABLE 7B
predicted ligand counts in SARX-CoV2 B.1.351 lineage (SA variant) background

| SLP | analysis setting | A01_count | A02_count | A03_count | A24_count | B07_count | B08_count |
|---|---|---|---|---|---|---|---|
| SLP 32 | SLP 32-1 in SA variant | 12 | 50 | 3 | 9 | 95 | 34 |
| | SLP 32 in SA variant | 0 | 1 | 2 | 0 | 90 | 0 |
| | intersection | +12 | +49 | +1 | +9 | +5 | +34 |
| SLP 15 | SLP 15-2 in SA variant | 73 | 4 | 3 | 82 | 45 | 0 |
| | SLP 15 in SA variant | 6 | 0 | 1 | 82 | 44 | 0 |
| | intersection | +67 | +4 | +2 | 0 | +2 | 0 |

TABLE 7C
predicted ligand counts in SARX-CoV2 P1 lineage (BR variant) background

| SLP | analysis setting | A01_count | A02_count | A03_count | A24_count | B07_count |
|---|---|---|---|---|---|---|
| SLP 15 | SLP 15-2 in BR variant | 73 | 4 | 3 | 82 | 46 |
| | SLP 15 in BR variant | 6 | 0 | 1 | 82 | 44 |
| | intersection | +67 | +4 | +2 | 0 | +2 |
| SLP 37 | SLP 37-1 in BR variant | 17 | 0 | 7 | 26 | 0 |
| | SLP 37 in BR variant | 1 | 0 | 5 | 26 | 0 |
| | intersection | +16 | 0 | +2 | 0 | 0 |

TABLE 7D
predicted ligand counts in SARX-CoV2 B.1.617 lineage (IN variant) background

| SLP | analysis setting | A01_count | A02_count | A03_count | A24_count | B07_count | B08_count |
|---|---|---|---|---|---|---|---|
| SLP 17 | SLP 17-1 in IN variant | 19 | 1 | 30 | 4 | 0 | 0 |
| | SLP 17 in IN variant | 1 | 1 | 0 | 0 | 0 | 0 |
| | intersection | +18 | 0 | +30 | +4 | 0 | 0 |
| SLP 12 | SLP 12-1 in IN variant | 19 | 0 | 40 | 6 | 0 | 0 |
| | SLP 12 in IN variant | 1 | 0 | 0 | 0 | 0 | 0 |
| | intersection | +18 | 0 | +40 | +6 | 0 | 0 |
| SLP 29 | SLP 29-1 in IN variant | 20 | 0 | 89 | 6 | 0 | 16 |
| | SLP 29 in IN variant | 2 | 0 | 56 | 0 | 0 | 16 |
| | intersection | +18 | 0 | +33 | +6 | 0 | 0 |
| SLP 15 | SLP 15-3 in UK variant | 7 | 0 | 2 | 79 | 47 | 0 |
| | SLP 15 in UK variant | 6 | 0 | 1 | 82 | 44 | 0 |
| | intersection | +1 | 0 | +1 | −3 | +3 | 0 |

N protein - SARS-CoV-2 UK variant
(SEQ ID NO: 332)
MSLNGPQNQRNAPRITFGGPSDSTGSNQNGERSGARSKQRRPQGLPNNTASWFTALTQHG
KEDLKFPRGQGVPINTNSSPDDQIGYYRRATRRIRGGDGKMKDLSPRWYFYYLGTGPEAGLP
YGANKDGIIWVATEGALNTPKDHIGTRNPANNAAIVLQLPQGTTLPKGFYAEGSRGGSQASS
RSSSRSRNSSRNSTPGSSKRTSPARMAGNGGDAALALLLLDRLNQLESKMFGKGQQQQGQ
TVTKKSAAEASKKPRQKRTATKAYNVTQAFGRRGPEQTQGNFGDQELIRQGTDYKHWPQIA
QFAPSASAFFGMSRIGMEVTPSGTWLTYTGAIKLDDKDPNFKDQVILLNKHIDAYKTFPPTEP
KKDKKKKADETQALPQRQKKQQTVTLLPAADLDDFSKQLQQSMSSADSTQA
S protein - SARS-CoV-2 UK variant
(SEQ ID NO: 333)
MFVFLVLLPLVSSQCVNLTTRTQLPPAYTNSFTRGVYYPDKVFRSSVLHSTQDLFLPFFSNVTW
FHAISGTNGTKRFDNPVLPFNDGVYFASTEKSNIIRGWIFGTTLDSKTQSLLIVNNATNVVIKV
CEFQFCNDPFLGVYHKNNKSWMESEFRVYSSANNCTFEYVSQPFLMDLEGKQGNFKNLREF
VFKNIDGYFKIYSKHTPINLVRDLPQGFSALEPLVDLPIGINITRFQTLLALHRSYLTPGDSSSG
WTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVS
PTKLNDLCFTNVYADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSYGFQPTYGVGYQPY
RVVVLSFELLHAPATVCGPKKSTNLVKNKCVNFNFNGLTGTGVLTESNKKFLPFQQFGRDIDD
TTDAVRDPQTLEILDITPCSFGGVSVITPGTNTSNQVAVLYQGVNCTEVPVAIHADQLTPTWR
VYSTGSNVFQTRAGCLIGAEHVNNSYECDIPIGAGICASYQTQTNSHRRARSVASQSIIAYTM
SLGAENSVAYSNNSIAIPINFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIEDLLFNK
VTLADAGFIKQYGDCLGDIAARDLICAQKFNGLTVLPPLLTDEMIAQYTSALLAGTITSGWTFG
AGAALQIPFAMQMAYRFNGIGVTQNVLYENQKLIANQFNSAIGKIQDSLSSTASALGKLQDV
VNQNAQALNTLVKQLSSNFGAISSVLNDILARLDKVEAEVQIDRLITGRLQSLQTYVTQQLIR
AAEIRASANLAATKMSECVLGQSKRVDFCGKGYHLMSFPQSAPHGVVFLHVTYVPAQEKNFT
TAPAICHDGKAHFPREGVFVSNGTHWFVTQRNFYEPQIITTHNTFVSGNCDVVIGIVNNTVYD
PLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVL
KGVKLHYT
N protein - SARS-CoV-2 SA variant
(SEQ ID NO: 334)
MSDNGPQNQRNAPRITFGGPSDSTGSNQNGERSGARSKQRRPQGLPNNTASWFTALTQHG
KEDLKFPRGQGVPINTNSSPDDQIGYYRRATRRIRGGDGKMKDLSPRWYFYYLGTGPEAGLP
YGANKDGIIWVATEGALNTPKDHIGTRNPANNAAIVLQLPQGTTLPKGFYAEGSRGGSQASS
RSSSRSRNSSRNSTPGSSRGISPARMAGNGGDAALALLLLDRLNQLESKMSGKGQQQQGQ
TVTKKSAAEASKKPRQKRTATKAYNVTQAFGRRGPEQTQGNFGDQELIRQGTDYKHWPQIA
QFAPSASAFFGMSRIGMEVTPSGTWLTYTGAIKLDDKDPNFKDQVILLNKHIDAYKTFPPTEP
KKDKKKKADETQALPQRQKKQQTVTLLPAADLDDFSKQLQQSMSSADSTQA
S protein - SARS-CoV-2 SA variant
(SEQ ID NO: 335)
MFVFLVLLPLVSSQCVNFTTRTQLPPAYTNSFTRGVYYPDKVFRSSVLHSTQDLFLPFFSNVTW
FHAIHVSGTNGTKRFANPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQGNFKNL
REFVFKNIDGYFKIYSKHTPINLVRGLPQGFSALEPLVDLPIGINITRFQTLHISYLTPGDSSSG
WTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVS
PTKLNDLCFTNVYADSFVIRGDEVRQIAPGQTGNIADYNYKLPDDFTGCVIAWNSNNLDSKV
GGNYNYLYRLFRKSNLKPFERDISTEIYQAGSTPCNGVKGFNCYFPLQSYGFQPTYGVGYQPY
RVVVLSFELLHAPATVCGPKKSTNLVKNKCVNFNFNGLTGTGVLTESNKKFLPFQQFGRDIAD
TTDAVRDPQTLEILDITPCSFGGVSVITPGTNTSNQVAVLYQGVNCTEVPVAIHADQLTPTWR
VYSTGSNVFQTRAGCLIGAEHVNNSYECDIPIGAGICASYQTQTNSPRRARSVASQSIIAYTM
SLGVENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIEDLLFNK
VTLADAGFIKQYGDCLGDIAARDLICAQKFNGLTVLPPLLTDEMIAQYTSALLAGTITSGWTFG
AGAALQIPFAMQMAYRFNGIGVTQNVLYENQKLIANQFNSAIGKIQDSLSSTASALGKLQDV
VNQNAQALNTLVKQLSSNFGAISSVLNDILSRLDKVEAEVQIDRLITGRLQSLQTYVTQQLIR
AAEIRASANLAATKMSECVLGQSKRVDFCGKGYHLMSFPQSAPHGVVFLHVTYVPAQEKNFT
TAPAICHDGKAHFPREGVFVSNGTHWFVTQRNFYEPQIITTDNTFVSGNCDVVIGIVNNTVYD
PLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVL
KGVKLHYT
N protein - SARS-CoV-2 BR variant
(SEQ ID NO: 336)
MSDNGPQNQRNAPRITFGGPSDSTGSNQNGERSGARSKQRRPQGLPNNTASWFTALTQHG
KEDLKFPRGQGVPINTNSSRDDQIGYYRRATRRIRGGDGKMKDLSPRWYFYYLGTGPEAGLP
YGANKDGIIWVATEGALNTPKDHIGTRNPANNAAIVLQLPQGTTLPKGFYAEGSRGGSQASS
RSSSRSRNSSRNSTPGSSKRTSPARMAGNGGDAALALLLLDRLNQLESKMSGKGQQQQGQ
TVTKKSAAEASKKPRQKRTATKAYNVTQAFGRRGPEQTQGNFGDQELIRQGTDYKHWPQIA
QFAPSASAFFGMSRIGMEVTPSGTWLTYTGAIKLDDKDPNFKDQVILLNKHIDAYKTFPPTEP
KKDKKKKADETQALPQRQKKQQTVTLLPAADLDDFSKQLQQSMSSADSTQA
S protein - SARS-CoV-2 BR variant
(SEQ ID NO: 337)
MFVFLVLLPLVSSQCVNFTNRTQLPSAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATNV
VIKVCEFQFCNYPFLGVYYHKNNKSWMESEFRVYSSANNCTFEYVSQPFLMDLEGKQGNFKN
LSEFVFKNIDGYFKIYSKHTPINLVRDLPQGFSALEPLVDLPIGINITRFQTLLALHRSYLTPGDS
SSGWTAGAAAYYVGYLQPRTFLLKYNENGTITDAVDCALDPLSETKCTLKSFTVEKGIYQTSN
FRVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGTIADYNYKLPDDFTGCVIAWNSNNLD
SKVGGNYNYLYRLFRKSNLKPFERDISTEIYQAGSTPCNGVKGFNCYFPLQSYGFQPTYGVGY
QPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQGVNCTEVPVAIHADQLTPT
WRVYSTGSNVFQTRAGCLIGAEYVNNSYECDIPIGAGICASYQTQTNSPRRARSVASQSIIAY
TMSLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDLLF
NKVTLADAGFIKQYGDCLGDIAARDLICAQKFNGLTVLPPLLTDEMIAQYTSALLAGTITSGWT
FGAGAALQIPFAMQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITGRLQSLQTYVTQQL
IRAAEIRASANLAAIKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVNNTV
YDPLQPELDSFKEELDKYFKNHTSPDVDLGDISGINASFVNIQKEIDRLNEVAKNLNESLIDLQ
ELGKYEQYIKWPWYIWLGFIAGLIAIVMVTIMLCCMTSCCSCLKGCCSCGSCCKFDEDDSEP
VLKGVKLHYT
N protein - SARS-CoV-2 IN variant
(SEQ ID NO: 338)
MSDNGPQNQRNAPRITFGGPSDSTGSNQNGERSGARSKQRRPQGLPNNTASWFTALTQHG
KEDLKFPRGQGVPINTNSSPDDQIGYYRRATRRIRGGDGKMKDLSPRWYFYYLGTGPEAGLP
YGANKDGIIWVATEGALNTPKDHIGTRNPANNAAIVLQLPQGTTLPKGFYAEGSRGGSQASS
RSSSRSRNSSRNSTPGSSMGTSPARMAGNGGDAALALLLLDRLNQLESKMSGKGQQQQGQ
TVTKKSAAEASKKPRQKRTATKAYNVTQAFGRRGPEQTQGNFGDQELIRQGTDYKHWPQIA
QFAPSASAFFGMSRIGMEVTPSGTWLTYTGAIKLDDKDPNFKDQVILLNKHIDAYKTFPPTEP
KKDKKKKAYETQALPQRQKKQQTVTLLPAADLDDFSKQLQQSMSSADSTQA
S protein - SARS-CoV-2 IN variant
(SEQ ID NO: 339)
MFVFLVLLPLVSSQCVNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQGNFKNL
REFVFKNIDGYFKIYSKHTPINLVRDLPQGFSALEPLVDLPIGINITRFQTLLALHRSYLTPGDSS
SGWTAGAAAYYVGYLQPRTFLLKYNENGTITDAVDCALDPLSETKCTLKSFTVEKGIYQTSNF
RVQPTESIVRFPNITNLCPFGEVFNATRFASVYAWNRKRISNCVADYSVLYNSASFSTFKCYG
VSPTKLNDLCFTNVYADSFVIRGDEVRQIAPGQTGKIADYNYKLPDDFTGCVIAWNSNNLDS
KVGGNYNYRYRLFRKSNLKPFERDISTEIYQAGSTPCNGVQGFNCYFPLQSYGFQPTNGVGY
QPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQGVNCTEVPVAIHADQLTPT
WRVYSTGSNVFQTRAGCLIGAEHVNNSYECDIPIGAGICASYQTQTNSRRRARSVASQSIIAY
TMSLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDLLF
NKVTLADAGFIKQYGDCLGDIAARDLICAQKFNGLTVLPPLLTDEMIAQYTSALLAGTITSGWT
FGAGAALQIPFAMQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITGRLQSLQTYVTQQL
IRAAEIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDDSEP
VLKGVKLHYT

1. An immunogenic peptide comprising a fragment of a SARS-CoV protein, wherein said fragment is 20-40 amino acids in length and wherein said fragment comprises one or more of the sequences set forth in:
o) the amino acid sequence LSPRWYFYY (SEQ ID NO:73) and one or more or all of the amino acid sequences: DLSPRWYFY (SEQ ID NO:202), DLSPRWYFYY (SEQ ID NO:203), GTGPEAGL (SEQ ID NO:204), MKDLSPRWYFY (SEQ ID NO:205), KDLSPRWYFY (SEQ ID NO:206), LSPRWYFYYL (SEQ ID NO:207), YLGTGPEA (SEQ ID NO:208), KMKDLSPRW (SEQ ID NO:209), KDLSPRWYFYY (SEQ ID NO:210), KMKDLSPRWY (SEQ ID NO:211), KMKDLSPRWYF (SEQ ID NO:212), KDLSPRWYF (SEQ ID NO:213) and SPRWYFYYLGT (SEQ ID NO:214),
i) the amino acid sequence ALNTLVKQL (SEQ ID NO:52) (S protein),
ii) the amino acid sequence VLNDILSRL (SEQ ID NO:53) (S protein),
iii) the amino acid sequence LITGRLQSL (SEQ ID NO:54) (S protein),
iv) the amino acid sequence QLIRAAEIRASANLAATK (SEQ ID NO:55) (S protein),
v) the amino acid sequence RLNEVAKNL (SEQ ID NO:56) (S protein),
vi) the amino acid sequence FIAGLIAIV (SEQ ID NO:57) (S protein),
vii) the amino acid sequence AYRFNGIGVTQNVLY (SEQ ID NO:58) (S protein),
viii) the amino acid sequence FLPFFSNVTWF (SEQ ID NO:59) (S protein),
ix) the amino acid sequence LLALHRSYL (SEQ ID NO:60) (S protein),
x) the amino acid sequence VRFPNITNL (SEQ ID NO:61) (S protein),
xi) the amino acid sequence IYQTSNFRVQ (SEQ ID NO:62) (S protein),
xii) the amino acid sequence SIIAYTMSLG (SEQ ID NO:63) (S protein),
xiii) the amino acid sequence IVNNATNVVIK (SEQ ID NO:64) (S protein),
xiv) the amino acid sequence LPNNTASW (SEQ ID NO:65) (N protein),
xv) the amino acid sequence ALNTPKDHI (SEQ ID NO:66) (N protein),
xvi) the amino acid sequence LQLPQGTTL (SEQ ID NO:67) (N protein),
xvii) the amino acid sequence LALLLLDRLNQL (SEQ ID NO:68) (N protein),
xviii) the amino acid sequence GMSRIGMEV (SEQ ID NO:69) (N protein),
xix) the amino acid sequence FTALTQHGK (SEQ ID NO:70) (N protein),
xx) the amino acid sequence FPRGQGVPI (SEQ ID NO:71) (N protein),
xxi) the amino acid sequence YYRRATRRIRG (SEQ ID NO:72) (N protein),
xxii) the amino acid sequence LSPRWYFYY (SEQ ID NO:73) (N protein),
xxiii) the amino acid sequence LALLLLDRLNQL (SEQ ID NO:74) (N protein),
xxiv) the amino acid sequence GMSRIGMEV (SEQ ID NO:75) (N protein),
xxv) the amino acid sequence ILLNKHID (SEQ ID NO:76) (N protein),
xxvi) the amino acid sequence KTFPPTEPK (SEQ ID NO:77) (N protein),
xxvii) the amino acid sequence NVTQAFGRRGP (SEQ ID NO:78) (N protein),
xxviii) the amino acid sequence KHWPQIAQFA (SEQ ID NO:79) (N protein),
xxix) the amino acid sequence TLACFVLAAV (SEQ ID NO:80) (M protein),
xxx) the amino acid sequence SFNPETNIL (SEQ ID NO:81) (M protein),
xxxi) the amino acid sequence TVATSRTLSY (SEQ ID NO:82) (M protein),
xxxii) the amino acid sequence IAWNSNNLDSK (SEQ ID NO:83) (RBD),
xxxiii) the amino acid sequence SKVGGNYNYLYRLFR (SEQ ID NO:84) (RBD),
xxxiv) the amino acid sequence CNGVEGFNC (SEQ ID NO:85) (RBD),
xxxv) the amino acid sequence NCYFPLQSYGFQPTNGVGY (SEQ ID NO:86) (RBD),
xxxvi) the amino acid sequence GNYNYLYRLFRKSNL (SEQ ID NO:87) (RBD),
xxxvii) the amino acid sequence set forth in SEQ ID NO:325 (S protein),
xxxviii) the amino acid sequence set forth in SEQ ID NO:331 (RBD), or
xxxix) the amino acid sequence set forth in SEQ ID NO:340 (RBD).

2. The immunogenic peptide according to claim 1, wherein said fragment comprises one or more of the sequences set forth in:
i) the amino acid sequence ALNTLVKQLSSN (SEQ ID NO:88) (S protein),
ii) the amino acid sequence VLNDILSRLDKV (SEQ ID NO:89) (S protein),
iii) the amino acid sequence LITGRLQSLQTY (SEQ ID NO:90) (S protein),
iv) the amino acid sequence QLIRAAEIRASANLAATK (SEQ ID NO:91) (S protein),
v) the amino acid sequence RLNEVAKNLNES (SEQ ID NO:92) (S protein),
vi) the amino acid sequence FIAGLIAIVMVT (SEQ ID NO:93) (S protein),
vii) the amino acid sequence AYRFNGIGVTQNVLY (SEQ ID NO:94) (S protein),
viii) the amino acid sequence FLPFFSNVTWFHAI (SEQ ID NO:95) (S protein),
ix) the amino acid sequence LLALHRSYLTPG (SEQ ID NO:96) (S protein),
x) the amino acid sequence VRFPNITNLCPF (SEQ ID NO:97) (S protein),
xi) the amino acid sequence IYQTSNFRVQPTE (SEQ ID NO:98) (S protein),
xii) the amino acid sequence SIIAYTMSLGAEN (SEQ ID NO:99) (S protein),
xiii) the amino acid sequence IVNNATNVVIKVCE (SEQ ID NO:100) (S protein),
xiv) the amino acid sequence LPNNTASWFTA (SEQ ID NO:101) (N protein),
xv) the amino acid sequence ALNTPKDHIGTR (SEQ ID NO:102) (N protein),
xvi) the amino acid sequence LQLPQGTTLPKG (SEQ ID NO:103) (N protein),
xvii) the amino acid sequence LALLLLDRLNQLESK (SEQ ID NO:104) (N protein),
xviii) the amino acid sequence GMSRIGMEVTPSGTWLTYT (SEQ ID NO:105) (N protein),
xix) the amino acid sequence FTALTQHGKEDL (SEQ ID NO:106) (N protein),
xx) the amino acid sequence FPRGQGVPINTN (SEQ ID NO:107) (N protein),
xxi) the amino acid sequence YYRRATRRIRGGDG (SEQ ID NO:108) (N protein),
xxii) the amino acid sequence LSPRWYFYYLGT (SEQ ID NO:109) (N protein),
xxiii) the amino acid sequence LALLLLDRLNQLESK (SEQ ID NO:110) (N protein),
xxiv) the amino acid sequence GMSRIGMEVTPS (SEQ ID NO:111) (N protein),
xxv) the amino acid sequence ILLNKHIDAYK (SEQ ID NO:112) (N protein),
xxvi) the amino acid sequence KTFPPTEPKKDK (SEQ ID NO:113) (N protein),
xxvii) the amino acid sequence NVTQAFGRRGPEQT (SEQ ID NO:114) (N protein),
xxviii) the amino acid sequence KHWPQIAQFAPSA (SEQ ID NO:115) (N protein),
xxix) the amino acid sequence TLACFVLAAVYRI (SEQ ID NO:116) (M protein),
xxx) the amino acid sequence SFNPETNILLNV (SEQ ID NO:117) (M protein),
xxxi) the amino acid sequence TVATSRTLSYYKL (SEQ ID NO:118) (M protein),
xxxii) the amino acid sequence IAWNSNNLDSKVGG (SEQ ID NO:119) (RBD),
xxxiii) the amino acid sequence SKVGGNYNYLYRLFR (SEQ ID NO:120) (RBD),
xxxiv) the amino acid sequence CNGVEGFNC (SEQ ID NO:121) (RBD),
xxxv) the amino acid sequence NCYFPLQSYGFQPTNGVGY (SEQ ID NO:122) (RBD),
xxxvi) the amino acid sequence GNYNYLYRLFRKSNL (SEQ ID NO:123) (RBD).

3. The immunogenic peptide according to claim 1, wherein said fragment comprises:
ii) the amino acid sequence VLNDILSRL and one or both of the amino acid sequences: SVLNDILSRL (SEQ ID NO:124) and SVLNDILSR (SEQ ID NO:125),
iii) the amino acid sequence LITGRLQSL and one or more or all of the amino acid sequences: ITGRLQSLQTY (SEQ ID NO:126), RLITGRLQSLQ (SEQ ID NO:127), LITGRLQSLQ (SEQ ID NO:128) and GRLQSLQTY (SEQ ID NO:129),
v) the amino acid sequence RLNEVAKNL and one or more or all of the amino acid sequences: RLNEVAKNLNE (SEQ ID NO:130), NLNESLIDL (SEQ ID NO:131), KEIDRLNEV (SEQ ID NO:132), and EVAKNLNESLI (SEQ ID NO:133),
vi) the amino acid sequence FIAGLIAIV and one or more or all of the amino acid sequences: GLIAIVMV (SEQ ID NO:134), QYIKWPWYI (SEQ ID NO:135), QYIKWPWYIW (SEQ ID NO:136), KWPWYIWLGF (SEQ ID NO:137), QYIKWPWYIWL (SEQ ID NO:138), KYEQYIKW (SEQ ID NO:139), KWPWYIWL (SEQ ID NO:140), IWLGFIAGL (SEQ ID NO:141), YIKWPWYIW (SEQ ID NO:142), WPWYIWLGFIA (SEQ ID NO:143) and YEQYIKWPW (SEQ ID NO:144),
vii) the amino acid sequence AYRFNGIGVTQNVLY and one or more or all of the amino acid sequences: LQIPFAMQM (SEQ ID NO:145), FAMQMAYRFNG (SEQ ID NO:146), QIPFAMQMAY (SEQ ID NO:147), IPFAMQMAY (SEQ ID NO:148), LQIPFAMQMAY (SEQ ID NO:149) and IPFAMQMAYRF (SEQ ID NO:150),
viii) the amino acid sequence FLPFFSNVTWF and one or more or all of the amino acid sequences: VTWFHAIHV (SEQ ID NO:151), LFLPFFSNVTW (SEQ ID NO:152), STQDLFLPFFS (SEQ ID NO:153), LPFFSNVTWFH (SEQ ID NO:154), NVTWFHAIHVS (SEQ ID NO:155), NVTWFHAIHV (SEQ ID NO:156), LPFFSNVT (SEQ ID NO:157) and FLPFFSNV (SEQ ID NO:158),
ix) the amino acid sequence LLALHRSYL and one or more or all of the amino acid sequences: TLLALHRSYLT (SEQ ID NO:159), NITRFQTLLAL (SEQ ID NO:160), TLLALHRSYL (SEQ ID NO:161), LPIGINITRFQ (SEQ ID NO:162), INITRFQTLL (SEQ ID NO:163), NITRFQTLLA (SEQ ID NO:164) and LPIGINIT (SEQ ID NO:165),
x) the amino acid sequence VRFPNITNL and one or more or all of the amino acid sequences: IYQTSNFRVQ (SEQ ID NO:166), KGIYQTSNFRV (SEQ ID NO:167) and QPTESIVRFPN (SEQ ID NO:168),
xi) the amino acid sequence IYQTSNFRVQ and one or both of the amino acid sequences: KGIYQTSNFRV (SEQ ID NO:169) and QPTESIVRFPN (SEQ ID NO:170),
xii) the amino acid sequence SIIAYTMSLG and one or more or all of the amino acid sequences: SQSIIAYTMSL (SEQ ID NO:171), SVAYSNNSI (SEQ ID NO:172) and LGAENSVAYSN (SEQ ID NO:173),
xiii) the amino acid sequence IVNNATNVVIK and one or more or all of the amino acid sequences: VIKVCEFQF (SEQ ID NO:174), NATNVVIKVCE (SEQ ID NO:175) and NVVIKVCEF (SEQ ID NO:176),
xiv) the amino acid sequence LPNNTASW and one or more or all of the amino acid sequences: FTALTQHGK (SEQ ID NO:177), NTASWFTALTQ (SEQ ID NO:178), GLPNNTASWFT (SEQ ID NO:179) and LPNNTASWFT (SEQ ID NO:180),
xv) the amino acid sequence ALNTPKDHI and one or both of the amino acid sequences: IIWVATEGA (SEQ ID NO:181) and ALNTPKDHIG (SEQ ID NO:182),
xvi) the amino acid sequence LQLPQGTTL and one or more or all of the amino acid sequences: GTTLPKGFY (SEQ ID NO:183), LQLPQGTTLPK (SEQ ID NO:184) and TTLPKGFYA (SEQ ID NO:185),
xvii) the amino acid sequence LALLLLDRLNQL and one or both of the amino acid sequences: ALALLLLDRLN (SEQ ID NO:186) and RLNQLESKMSG (SEQ ID NO:187),
xviii) the amino acid sequence GMSRIGMEVTPSGTWL and one or more or all of the amino acid sequences: VTPSGTWLTYT (SEQ ID NO:188), TWLTYTGAI (SEQ ID NO:189), EVTPSGTWLT (SEQ ID NO:190) and TPSGTWLTY (SEQ ID NO:191),
xix) the amino acid sequence FTALTQHGK and one or more or all of the amino acid sequences: NTASWFTALTQ (SEQ ID NO:192), FPRGQGVPI (SEQ ID NO:193) and LPNNTASW (SEQ ID NO:194),
xx) the amino acid sequence FPRGQGVPI and one or more or all of the amino acid sequences: NTASWFTALTQ (SEQ ID NO:195), FTALTQHGK (SEQ ID NO:196) and LPNNTASW (SEQ ID NO:197),
xxi) the amino acid sequence YYRRATRRIRG and one or more or all of the amino acid sequences: SSPDDQIG (SEQ ID NO:198), IGYYRRATR (SEQ ID NO:199), GYYRRATRRIR (SEQ ID NO:200) and SPDDQIGYY (SEQ ID NO:201),
xxii) the amino acid sequence LSPRWYFYY and one or more or all of the amino acid sequences: DLSPRWYFY (SEQ ID NO:202), DLSPRWYFYY (SEQ ID NO:203), GTGPEAGL (SEQ ID NO:204), MKDLSPRWYFY (SEQ ID NO:205), KDLSPRWYFY (SEQ ID NO:206), LSPRWYFYYL (SEQ ID NO:207), YLGTGPEA (SEQ ID NO:208), KMKDLSPRW (SEQ ID NO:209), KDLSPRWYFYY (SEQ ID NO:210), KMKDLSPRWY (SEQ ID NO:211), KMKDLSPRWYF (SEQ ID NO:212), KDLSPRWYF (SEQ ID NO:213) and SPRWYFYYLGT (SEQ ID NO:214),
xxiii) the amino acid sequence LALLLLDRLNQL and one or both of the amino acid sequences: ALALLLLDRLN (SEQ ID NO:215) and RLNQLESKMSG (SEQ ID NO:216),
xxiv) the amino acid sequence GMSRIGMEV and one or more or all of the amino acid sequences: VTPSGTWLTYT (SEQ ID NO:217), EVTPSGTWL (SEQ ID NO:218), GMSRIGMEVTP (SEQ ID NO:219), EVTPSGTWLT (SEQ ID NO:220), TPSGTWLTY (SEQ ID NO:221) and MEVTPSGTWL (SEQ ID NO:222),
xxv) the amino acid sequence ILLNKHID and one or more or all of the amino acid sequences: LLNKHIDAY (SEQ ID NO:223), KTFPPTEPKKD (SEQ ID NO:224), LLNKHIDAYK (SEQ ID NO:225), KTFPPTEP (SEQ ID NO:226), KHIDAYKTF (SEQ ID NO:227) and VILLNKHIDAY (SEQ ID NO:228),
xxvi) the amino acid sequence KTFPPTEPK and one or more or all of the amino acid sequences: LLNKHIDAY (SEQ ID NO:229), KTFPPTEPKKD (SEQ ID NO:230), LLNKHIDAYK (SEQ ID NO:231), KHIDAYKTF (SEQ ID NO:232), VILLNKHIDAY (SEQ ID NO:233), and ILLNKHID (SEQ ID NO:234),
xxviii) the amino acid sequence KHWPQIAQFA and one or both of the amino acid sequences: QFAPSASA (SEQ ID NO:235) and RQGTDYKHW (SEQ ID NO:236),
xxix) the amino acid sequence TLACFVLAAV and one or more or all of the amino acid sequences: TLACFVLA (SEQ ID NO:237), WLLWPVTLA (SEQ ID NO:238), FVLAAVYRI (SEQ ID NO:239), LWLLWPVTL (SEQ ID NO:240), LWPVTLACF (SEQ ID NO:241), ACFVLAAVYRI (SEQ ID NO:242) and WPVTLACFVL (SEQ ID NO:243),
xxx) the amino acid sequence SFNPETNIL and one or more or all of the amino acid sequences: SMWSFNPETN (SEQ ID NO:244), FNPETNILL (SEQ ID NO:245), LFARTRSMW (SEQ ID NO:246), LFARTRSMWSF (SEQ ID NO:247), RTRSMWSFNP (SEQ ID NO:248), ARTRSMWSFNP (SEQ ID NO:249), FARTRSMW (SEQ ID NO:250) and FARTRSMWSF (SEQ ID NO:251),
xxxi) the amino acid sequence TVATSRTLSY and one or more or all of the amino acid sequences: VATSRTLSYY (SEQ ID NO:252), ATSRTLSYYKL (SEQ ID NO:253), ITVATSRTLSY (SEQ ID NO:254), VATSRTLSY (SEQ ID NO:255), TVATSRTLSYY (SEQ ID NO:256), ATSRTLSYYK (SEQ ID NO:257), ATSRTLSY (SEQ ID NO:258), VATSRTLSYYK (SEQ ID NO:259), TSRTLSYYKLG (SEQ ID NO:260), SRTLSYYKLGA (SEQ ID NO:261), ITVATSRTL (SEQ ID NO:262) and LPKEITVATSR (SEQ ID NO:263),
xxxii) the amino acid sequence IAWNSNNLDSK, and one or more or all of the amino acid sequences: GCVIAWNSNNLDSKV (SEQ ID NO:264), KVGGNYNYLYRLFRK (SEQ ID NO:265), VGGNYNYLYRLFRKS (SEQ ID NO:266), GGNYNYLYRLFRKSN (SEQ ID NO:267), KVGGNYNYLY (SEQ ID NO:268), VIAWNSNNL (SEQ ID NO:269), KVGGNYNYL (SEQ ID NO:270), NYNYLYRLF (SEQ ID NO:271), SKVGGNYNYLY (SEQ ID NO:272), VGGNYNYLY (SEQ ID NO:273), NYNYLYRLFR (SEQ ID NO:274), YNYLYRLFR (SEQ ID NO:275) and YLYRLFRKS (SEQ ID NO:276),
xxxiii) the amino acid sequence SKVGGNYNYLYRLFR, and one or more or all of the amino acid sequences: KVGGNYNYLYRLFRK (SEQ ID NO:277), VGGNYNYLYRLFRKS (SEQ ID NO:278), GGNYNYLYRLFRKSN (SEQ ID NO:279), GNYNYLYRLFRKSNL (SEQ ID NO:280), NYNYLYRLFRKSNLK (SEQ ID NO:281), GCVIAWNSNNLDSKV (SEQ ID NO:282), KVGGNYNYLY (SEQ ID NO:283), KVGGNYNYL (SEQ ID NO:284), NYNYLYRLF (SEQ ID NO:285), SKVGGNYNYLY (SEQ ID NO:286), VGGNYNYLY (SEQ ID NO:287), NYNYLYRLFR (SEQ ID NO:288), YNYLYRLFR (SEQ ID NO:289), YLYRLFRKS (SEQ ID NO:290) and VIAWNSNNL (SEQ ID NO:291),
xxxiv) the amino acid sequence CNGVEGFNC, and one or more or all of the amino acid sequences: NGVEGFNCYFPLQSY (SEQ ID NO:292), GVEGFNCYFPLQSYG (SEQ ID NO:293), VEGFNCYFPLQSYGF (SEQ ID NO:294), EGFNCYFPLQSYGFQ (SEQ ID NO:295), GFNCYFPLQSYGFQP (SEQ ID NO:296), CYFPLQSY (SEQ ID NO:297), YFPLQSYGFQP (SEQ ID NO:298), NCYFPLQSYGF (SEQ ID NO:299) and FPLQSYGFQP (SEQ ID NO:300),
xxxv) the amino acid sequence NCYFPLQSYGFQPTNGVGY and one or more or all of the amino acid sequences: NGVEGFNCYFPLQSY (SEQ ID NO:301), GVEGFNCYFPLQSYG (SEQ ID NO:302), VEGFNCYFPLQSYGF (SEQ ID NO:303), EGFNCYFPLQSYGFQ (SEQ ID NO:304) and GFNCYFPLQSYGFQP (SEQ ID NO:305),
xxxvi) the amino acid sequence GNYNYLYRLFRKSNL and one or more or all of the amino acid sequences: KVGGNYNYLYRLFRK (SEQ ID NO:306), VGGNYNYLYRLFRKS (SEQ ID NO:307), GGNYNYLYRLFRKSN (SEQ ID NO:308), GNYNYLYRLFRKSNL (SEQ ID NO:309), NYNYLYRLFRKSNLK (SEQ ID NO:310), YNYLYRLFRKSNLKP (SEQ ID NO:311), NYLYRLFRKSNLKPF (SEQ ID NO:312), YLYRLFRKSNLKPFE (SEQ ID NO:313), LYRLFRKSNLKPFER (SEQ ID NO:314), YRLFRKSNLKPFERD (SEQ ID NO:315), KVGGNYNYLY (SEQ ID NO:316), KVGGNYNYL (SEQ ID NO:317), RLFRKSNLKP (SEQ ID NO:318), SKVGGNYNYLY (SEQ ID NO:319), VGGNYNYLY (SEQ ID NO:320), KSNLKPFER (SEQ ID NO:321) and YRLFRKSNLKP (SEQ ID NO:322).

4. The immunogenic peptide according to claim 1, wherein said peptide consists of said fragment of a SARS-CoV protein.

5. The immunogenic peptide according to claim 1, wherein said peptide is 20-40 amino acids in length, such as 20, 21, 22, 23, 24, 25, 26, 27, 28, 29, 30, 31, 32, 33, 34, 35, 36, 37, 38, 39 or 40 amino acids length, e.g. 20-34 amino acids in length, such as 20-33 amino acids in length, e.g. 20-32 amino acids in length, such as 20-31 amino acids in length, e.g. 20-30 amino acids in length, such as 20-29 amino acids in length, e.g. 20-28 amino acids in length, such as 20-27 amino acids in length, e.g. 20-26 or 20-25 amino acids in length.

6. The immunogenic peptide according to claim 1, wherein said fragment is 20-40 amino acids in length, such as 20, 21, 22, 23, 24, 25, 26, 27, 28, 29, 30, 31, 32, 33, 34, 35, 36, 37, 38, 39 or 40 amino acids length, e.g. 20-34 amino acids in length, such as 20-33 amino acids in length, e.g. 20-32 amino acids in length, such as 20-32 amino acids in length, such as 20-31 amino acids in length, e.g. 20-30 amino acids in length, such as 20-29 amino acids in length, e.g. 20-28 amino acids in length, such as 20-27 amino acids in length, e.g. 20-26 or 20-25 amino acids in length.

7. The immunogenic peptide according to claim 1, wherein the peptide comprises or consists of a sequence selected from the group consisting of: SEQ ID NO:1-45 or SEQ ID NO:326 or SEQ ID NO:327 or SEQ ID NO:328 or SEQ ID NO:330 or SEQ ID NO:342 or SEQ ID NO:343.

8. The immunogenic peptide according to claim 7, wherein the immunogenic peptide comprises or consist of a sequence selected from the group consisting of: SEQ ID NO: 5, 9, 10, 12, 15, 17, 19, 22, 24, 29, 30, 32, 33, 36, 37, 38, 39, 40, 44 and 45, preferably wherein the immunogenic peptide comprises or consist of a sequence selected from the group consisting of: SEQ ID NO:9, 10, 12, 15, 19, 22, 32, 33, 37, 38, 39 and 40, more preferably wherein the immunogenic peptide comprises or consist of a sequence selected from the group consisting of: SEQ ID NO:9, 19, 32, 33, 38 and 39, most preferably wherein the immunogenic peptide comprises or consist of a sequence selected from the group consisting of: SEQ ID NO:19, 22 and 38.

9. The immunogenic peptide according to claim 1, wherein the immunogenic peptide comprises or consists of the sequence set forth in SEQ ID NO:323.

10. A polynucleotide encoding an immunogenic peptide according to claim 1, wherein optionally the sequence encoding the immunogenic peptide is part of a larger open reading frame also containing flanking amino acids, provided that such flanking amino acids are not contiguous with the immunogenic peptide sequence in the SARS-CoV protein from which the immunogenic peptide is derived.

11. An immunogenic composition comprising one or more immunogenic peptides according to claim 1 and a pharmaceutically-acceptable carrier.

12. A vaccine product comprising one or more immunogenic compositions, wherein the one or more immunogenic compositions comprise:
an immunogenic peptide as defined in claim 1 (xxx) and an immunogenic peptide as defined in claim 1 (xxii), or
an immunogenic peptide as defined in claim 1 (xxx) and an immunogenic peptide as defined in claim 1 (xxviii), or
an immunogenic peptide as defined in claim 1 (xxii) and an immunogenic peptide as defined in claim 1 (xxviii), or
an immunogenic peptide as defined in claim 1 (xxx), and an immunogenic peptide as defined in claim 1 (xxii), and an immunogenic peptide as defined in claim 1 (xxviii), or
two, three, four, five, six or all seven of: the immunogenic peptides as defined in claim 1 (ii), claim 1 (xxxiii), claim 1 (xxx), claim 1 (ix), claim 1 (x), claim 1 (xxi) and claim 1 (xxii), or
two, three, four, five, six, seven or all eight of: the immunogenic peptides as defined in claim 1 (ii), claim 1 (xxxiii), claim 1 (xxx), claim 1 (ix), claim 1 (x), claim 1 (xxi), claim 1 (xxii) and claim 1 (xxviii),
wherein the immunogenic peptides optionally are distributed over two or three immunogenic compositions.

13. A vaccine product comprising one or more immunogenic compositions, wherein the one or more immunogenic compositions comprise the peptides set forth in:
SEQ ID NO:19 and SEQ ID NO:38, or
SEQ ID NO:19 and SEQ ID NO:323, or
SEQ ID NO:38 and SEQ ID NO:323, or
SEQ ID NO:19, SEQ ID NO:38 and SEQ ID NO:323, or
two, three or all of SEQ ID NO:19, 22, 38 and 323, or
two, three, four, five, six or all of: SEQ ID NO:9, 12, 19, 32, 33, 37 and 38, or
two, three, four, five, six or all of: SEQ ID NO:326, 12, 19, 32, 33, 37 and 38, or
two, three, four, five, six or all of: SEQ ID NO:9, 12, 19, 325, 33, 37 and 38, or
two, three, four, five, six or all of: SEQ ID NO:9, 12, 19, 32, 33, 328 and 38, or
two, three, four, five, six or all of: SEQ ID NO:9, 340, 19, 32, 33, 37 and 38, or
two, three, four, five, six, seven or all of: SEQ ID NO:9, 12, 19, 32, 33, 37, 38 and 323, or
two, three, four, five, six, seven or all of: SEQ ID NO:326, 12, 19, 32, 33, 37, 38 and 323, or
two, three, four, five, six, seven or all of: SEQ ID NO:9, 12, 19, 325, 33, 37, 38 and 323, or
two, three, four, five, six, seven or all of: SEQ ID NO:9, 12, 19, 32, 33, 328, 38 and 323, or
two, three, four, five, six, seven or all of: SEQ ID NO:9, 340, 19, 32, 33, 37, 38 and 323, or
two, three, four, five, six, seven, eight, nine or all of: SEQ ID NO:9, 12, 19, 22, 32, 33, 37, 38, 39 and 40, or
two, three, four, five, six, seven, eight, nine or all of: SEQ ID NO:326, 12, 19, 22, 32, 33, 37, 38, 39 and 40, or
two, three, four, five, six, seven, eight, nine or all of: SEQ ID NO:9, 12, 19, 22, 325, 33, 37, 38, 39 and 40, or
two, three, four, five, six, seven, eight, nine or all of: SEQ ID NO:9, 12, 19, 22, 32, 33, 328, 38, 39 and 40, or
two, three, four, five, six, seven, eight, nine or all of: SEQ ID NO:9, 340, 19, 22, 32, 33, 37, 38, 39 and 40, or
two, three, four, five, six, seven, eight, nine, ten or all of: SEQ ID NO:9, 12, 19, 22, 32, 33, 37, 38, 39, 40 and 323, or
two, three, four, five, six, seven, eight, nine, ten or all of: SEQ ID NO:326, 12, 19, 22, 32, 33, 37, 38, 39, 40 and 323, or
two, three, four, five, six, seven, eight, nine, ten or all of: SEQ ID NO:9, 12, 19, 22, 325, 33, 37, 38, 39, 40 and 323, or
two, three, four, five, six, seven, eight, nine, ten or all of: SEQ ID NO:9, 12, 19, 22, 32, 33, 328, 38, 39, 40 and 323, or
two, three, four, five, six, seven, eight, nine, ten or all of: SEQ ID NO:9, 340, 19, 22, 32, 33, 37, 38, 39, 40 and 323, or
two, three, four, five, six, seven, eight, nine, ten, eleven, twelve or all of: SEQ ID NO: 9, 10, 12, 15, 19, 22, 32, 33, 37, 38, 39, 40 and 323, or
two, three, four, five, six, seven, eight, nine, ten, eleven, twelve or all of: SEQ ID NO: 326, 327, 12, 330, 19, 22, 32, 33, 37, 38, 39, 40 and 323, or
two, three, four, five, six, seven, eight, nine, ten, eleven, twelve or all of: SEQ ID NO: 9, 10, 12, 331, 19, 22, 325, 33, 37, 38, 39, 40 and 323, or
two, three, four, five, six, seven, eight, nine, ten, eleven, twelve or all of: SEQ ID NO: 9, 10, 12, 331, 19, 22, 32, 33, 328, 38, 39, 40 and 323, or
two, three, four, five, six, seven, eight, nine, ten, eleven, twelve or all of: SEQ ID NO: 9, 10, 340, 15, 19, 22, 32, 33, 37, 38, 39, 40 and 323, or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 12, 15, 17, 19, 22, 24, 29, 30, 32, 33, 36, 37, 38, 39, 40, 44, 45 and 323, or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 340, 15, 342, 19, 22, 24, 343, 30, 32, 33, 36, 37, 38, 39, 40, 44, 45 and 323, or
two, three, four, five or all of: SEQ ID NO:9, 19, 32, 33, 38 and 39, or
two, three, four, five or all of: SEQ ID NO:326, 19, 32, 33, 38 and 39, or
two, three, four, five or all of: SEQ ID NO:9, 19, 325, 33, 38 and 39.

14. A vaccine product comprising one or more immunogenic compositions, wherein the one or more immunogenic compositions comprise one or more polynucleotides according to claim 10.

15. A method for the treatment, such as therapeutic treatment or prevention, of a SARS-CoV-related disease comprising administration to a human subject of one or more of the immunogenic peptides defined in claim 1.

16. The method according to claim 15, comprising administration of 2 or more, e.g. 3 or more, such as 4 or more, e.g. 5 or more, such as 6 or more, e.g. 7 or more, such as 8 or more, e.g. 9 or more, such as 10 or more, e.g. 11 or more, such as 12 or more of the immunogenic peptides defined in claim 1(i) to 1(xxxi).

17. The method according to claim 15, comprising administration of:
the immunogenic peptide defined in claim 1 (i) and the immunogenic peptide defined in claim 1 (ii), or
the immunogenic peptide defined in claim 1 (i) and the immunogenic peptide defined in claim 1 (v), or
the immunogenic peptide defined in claim 1 (i) and the immunogenic peptide defined in claim 1 (xvi), or
the immunogenic peptide defined in claim 1 (ii) and the immunogenic peptide defined in claim 1 (v), or
the immunogenic peptide defined in claim 1 (ii) and the immunogenic peptide defined in claim 1 (xvi), or
the immunogenic peptide defined in claim 1 (v) and the immunogenic peptide defined in claim 1 (xvi), or
the immunogenic peptide defined in claim 1 (i), the immunogenic peptide defined in claim 1 (ii) and the immunogenic peptides defined in claim 1 (v), or
the immunogenic peptide defined in claim 1 (i), the immunogenic peptide defined in claim 1 (ii) and the immunogenic peptides defined in claim 1 (xvi), or
the immunogenic peptide defined in claim 1 (i), the immunogenic peptide defined in claim 1 (v) and the immunogenic peptides defined in claim 1 (xvi), or
the immunogenic peptide defined in claim 1 (ii), the immunogenic peptide defined in claim 1 (v) and the immunogenic peptides defined in claim 1 (xvi), or
the immunogenic peptide defined in claim 1 (i), the immunogenic peptide defined in claim 1 (ii), the immunogenic peptides defined in claim 1 (v), and the immunogenic peptides defined in claim 1 (xvi), or
the immunogenic peptide defined in claim 1 (xix) and the immunogenic peptide defined in claim 1 (xx), or
the immunogenic peptide defined in claim 1 (xix) and the immunogenic peptide defined in claim 1 (viii), or
the immunogenic peptide defined in claim 1 (xix) and the immunogenic peptide defined in claim 1 (xxxiv), or
the immunogenic peptide defined in claim 1 (xx) and the immunogenic peptide defined in claim 1 (viii), or
the immunogenic peptide defined in claim 1 (xx) and the immunogenic peptide defined in claim 1 (xxxiv), or
the immunogenic peptide defined in claim 1 (viii) and the immunogenic peptide defined in claim 1 (xxxiv), or
the immunogenic peptide defined in claim 1 (xix), the immunogenic peptide defined in claim 1 (xx) and the immunogenic peptides defined in claim 1 (viii), or
the immunogenic peptide defined in claim 1 (xix), the immunogenic peptide defined in claim 1 (xx) and the immunogenic peptides defined in claim 1 (xxxiv), or
the immunogenic peptide defined in claim 1 (xix), the immunogenic peptide defined in claim 1 (viii) and the immunogenic peptides defined in claim 1 (xxxiv), or
the immunogenic peptide defined in claim 1 (xx), the immunogenic peptide defined in claim 1 (viii) and the immunogenic peptides defined in claim 1 (xxxiv), or
the immunogenic peptide defined in claim 1 (xix), the immunogenic peptide defined in claim 1 (xx), the immunogenic peptides defined in claim 1 (viii), and the immunogenic peptides defined in claim 1 (xxxiv).

18. The method according to claim 15, comprising administration of:
an immunogenic peptide as defined in claim 1 (xxx) and an immunogenic peptide defined in claim 1 (xxii), or
an immunogenic peptide as defined in claim 1 (xxx) and an immunogenic peptide as defined in claim 1 (xxviii), or
an immunogenic peptide as defined in claim 1 (xxii) and an immunogenic peptide as defined in claim 1 (xxviii), or.

19. The method according to claim 18, comprising administration of the peptides set forth in:
SEQ ID NO:19 and SEQ ID NO:38, or
SEQ ID NO:19 and SEQ ID NO:323, or
SEQ ID NO:38 and SEQ ID NO:323, or
SEQ ID NO:19, SEQ ID NO:38 and SEQ ID NO:323, or
two, three or all of SEQ ID NO:19, 22, 38 and 323, or
two, three, four, five, six or all of: SEQ ID NO:9, 12, 19, 32, 33, 37 and 38, or
two, three, four, five, six or all of: SEQ ID NO:326, 12, 19, 32, 33, 37 and 38, or
two, three, four, five, six or all of: SEQ ID NO:9, 12, 19, 325, 33, 37 and 38, or
two, three, four, five, six or all of: SEQ ID NO:9, 12, 19, 32, 33, 328 and 38, or
two, three, four, five, six or all of: SEQ ID NO:9, 340, 19, 32, 33, 37 and 38, or
two, three, four, five, six, seven or all of: SEQ ID NO:9, 12, 19, 32, 33, 37, 38 and 323, or
two, three, four, five, six, seven or all of: SEQ ID NO:326, 12, 19, 32, 33, 37, 38 and 323, or
two, three, four, five, six, seven or all of: SEQ ID NO:9, 12, 19, 325, 33, 37, 38 and 323, or
two, three, four, five, six, seven or all of: SEQ ID NO:9, 12, 19, 32, 33, 328, 38 and 323, or
two, three, four, five, six, seven or all of: SEQ ID NO:9, 340, 19, 32, 33, 37, 38 and 323, or
two, three, four, five, six, seven, eight, nine or all of: SEQ ID NO:9, 12, 19, 22, 32, 33, 37, 38, 39 and 40, or
two, three, four, five, six, seven, eight, nine or all of: SEQ ID NO:326, 12, 19, 22, 32, 33, 37, 38, 39 and 40, or
two, three, four, five, six, seven, eight, nine or all of: SEQ ID NO:9, 12, 19, 22, 325, 33, 37, 38, 39 and 40, or
two, three, four, five, six, seven, eight, nine or all of: SEQ ID NO:9, 12, 19, 22, 32, 33, 328, 38, 39 and 40, or
two, three, four, five, six, seven, eight, nine or all of: SEQ ID NO:9, 340, 19, 22, 32, 33, 37, 38, 39 and 40, or two, three, four, five, six, seven, eight, nine, ten or all of: SEQ ID NO:9, 12, 19, 22, 32, 33, 37, 38, 39, 40 and 323, or
two, three, four, five, six, seven, eight, nine, ten or all of: SEQ ID NO:326, 12, 19, 22, 32, 33, 37, 38, 39, 40 and 323, or
two, three, four, five, six, seven, eight, nine, ten or all of: SEQ ID NO:9, 12, 19, 22, 325, 33, 37, 38, 39, 40 and 323, or
two, three, four, five, six, seven, eight, nine, ten or all of: SEQ ID NO:9, 12, 19, 22, 32, 33, 328, 38, 39, 40 and 323, or
two, three, four, five, six, seven, eight, nine, ten or all of: SEQ ID NO:9, 340, 19, 22, 32, 33, 37, 38, 39, 40 and 323, or
two, three, four, five, six, seven, eight, nine, ten, eleven, twelve or all of: SEQ ID NO: 9, 10, 12, 15, 19, 22, 32, 33, 37, 38, 39, 40 and 323, or
two, three, four, five, six, seven, eight, nine, ten, eleven, twelve or all of: SEQ ID NO: 326, 327, 12, 330, 19, 22, 32, 33, 37, 38, 39, 40 and 323, or
two, three, four, five, six, seven, eight, nine, ten, eleven, twelve or all of: SEQ ID NO: 9, 10, 12, 331, 19, 22, 325, 33, 37, 38, 39, 40 and 323, or
two, three, four, five, six, seven, eight, nine, ten, eleven, twelve or all of: SEQ ID NO: 9, 10, 12, 331, 19, 22, 32, 33, 328, 38, 39, 40 and 323, or
two, three, four, five, six, seven, eight, nine, ten, eleven, twelve or all of: SEQ ID NO: 9, 10, 340, 15, 19, 22, 32, 33, 37, 38, 39, 40 and 323, or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 12, 15, 17, 19, 22, 24, 29, 30, 32, 33, 36, 37, 38, 39, 40, 44, 45 and 323, or
two, three, four, five, six, seven, eight, nine, ten, eleven, twelve, thirteen, fourteen, fifteen, sixteen, seventeen, eighteen, nineteen, twenty or all of: SEQ ID NO: 5, 9, 10, 340, 15, 342, 19, 22, 24, 343, 30, 32, 33, 36, 37, 38, 39, 40, 44, 45 and 323, or
two, three, four, five or all of: SEQ ID NO:9, 19, 32, 33, 38 and 39, or
two, three, four, five or all of: SEQ ID NO:326, 19, 32, 33, 38 and 39, or
two, three, four, five or all of: SEQ ID NO:9, 19, 325, 33, 38 and 39.

20. The method according to claim 15, comprising administration of peptides derived from two or all three of: S protein, N protein, M protein.

21. The method according to claim 15, comprising administration of 2, 3, 4, 5, 6, 7, 8, 9, 10 or all of the peptides selected from the group consisting of:
the immunogenic peptide defined in claim 1 (i),
the immunogenic peptide defined in claim 1 (ii),
the immunogenic peptide defined in claim 1 (iii),
the immunogenic peptide defined in claim 1 (iv),
the immunogenic peptide defined in claim 1 (v),
the immunogenic peptide defined in claim 1 (vii),
the immunogenic peptide defined in claim 1 (xiv),
the immunogenic peptide defined in claim 1 (xvi),
the immunogenic peptide defined in claim 1 (xxvi),
the immunogenic peptide defined in claim 1 (xxvii), and
the immunogenic peptide defined in claim 1 (xxviii).

22. The method according to claim 15, further comprising administration of an adjuvant, wherein said adjuvant preferably is
a water-in-oil emulsion comprising a mineral oil and a surfactant from the mannide monooleate family, optionally combined a TLR9 agonist, or
a TLR2 ligand.

23. The method according to claim 15, wherein the composition(s) are administered more than once, and wherein the second administration is performed after less than 28 days after the first administration, such as less than 21 days, e.g. after between 5 and 20 days, such as after 5, 7, 10 or 14 days.

24. A method for the treatment, such as therapeutic treatment or prevention, of a SARS-CoV-related disease comprising administration to a human subject of one or more polynucleotides according to claim 10.

25. A method for selecting a peptide sequence for use in immunization against a SARS-CoV-related disease, comprising the steps of:
a) identify S, N and/or M variant protein sequences of a variant SARS-CoV strain circulating in the human population,
b) compare the reference peptide sequences described in any one of the preceding claims with the corresponding variant protein sequences, identify amino acid sequence differences and design a variant peptide sequence incorporating said amino acid difference(s),
c) analyze the effect of said amino acid sequence differences on predicted ligands by:
i) determining predicted ligand counts per HLA super-type for the reference peptide sequence in the variant background and for the variant peptide sequence in the variant background,
ii) assessing whether there is an increased total number of predicted ligands when using the variant peptide sequence in the variant background as compared to the number obtained with reference peptide sequence in the variant background,
d) select the variant peptide sequence for use in immunization if an increased number of predicted ligands is obtained in step c) ii).

26. A method for producing an immunogenic peptide, said method comprising performing steps a), b), c) and d) according to claim 25 and a further step of producing said variant peptide.

27. The method according to claim 26, comprising a further step of combining said variant peptide with one or more further immunogenic peptides into a vaccine product or for combined use in immunization, wherein the one or more further immunogenic peptides do not include the reference peptide.


SEQUENCE LISTING (455 sequences)

SEQ ID NO: 1 - protein, 22 aa, Unknown
  SARS-CoV sequence
NAQALNTLVKQLSSNFGAISSV

SEQ ID NO: 2 - protein, 25 aa, Unknown
  SARS-CoV sequence
LSSNFGAISSVLNDILSRLDKVEAE

SEQ ID NO: 3 - protein, 23 aa, Unknown
  SARS-CoV sequence
DKVEAEVQIDRLITGRLQSLQTY

SEQ ID NO: 4 - protein, 23 aa, Unknown
  SARS-CoV sequence
TQQLIRAAEIRASANLAATKMSE

SEQ ID NO: 5 - protein, 25 aa, Unknown
  SARS-CoV sequence
KEIDRLNEVAKNLNESLIDLQELGK

SEQ ID NO: 6 - protein, 20 aa, Unknown
  SARS-CoV sequence
WPWYIWLGFIAGLIAIVMVT

SEQ ID NO: 7 - protein, 33 aa, Unknown
  SARS-CoV sequence
DLQELGKYEQYIKWPWYIWLGFIAGLIAIVMVT

SEQ ID NO: 8 - protein, 29 aa, Unknown
  SARS-CoV sequence
ALQIPFAMQMAYRFNGIGVTQNVLYENQK

SEQ ID NO: 9 - protein, 30 aa, Unknown
  SARS-CoV sequence
ALNTLVKQLSSNFGAISSVLNDILSRLDKV

SEQ ID NO: 10 - protein, 32 aa, Unknown
  SARS-CoV sequence
VLNDILSRLDKVEAEVQIDRLITGRLQSLQTY

SEQ ID NO: 11 - protein, 28 aa, Unknown
  SARS-CoV sequence
TGCVIAWNSNNLDSKVGGNYNYLYRLFR

SEQ ID NO: 12 - protein, 25 aa, Unknown
  SARS-CoV sequence
SNNLDSKVGGNYNYLYRLFRKSNLK

SEQ ID NO: 13 - protein, 23 aa, Unknown
  SARS-CoV sequence
TEIYQAGSTPCNGVEGFNCYFPL

SEQ ID NO: 14 - protein, 23 aa, Unknown
  SARS-CoV sequence
FNCYFPLQSYGFQPTNGVGYQPY

SEQ ID NO: 15 - protein, 29 aa, Unknown
  SARS-CoV sequence
STPCNGVEGFNCYFPLQSYGFQPTNGVGY

SEQ ID NO: 16 - protein, 34 aa, Unknown
  SARS-CoV sequence
AGSTPCNGVEGFNCYFPLQSYGFQPTNGVGYQPY

SEQ ID NO: 17 - protein, 31 aa, Unknown
  SARS-CoV sequence
TGCVIAWNSNNLDSKVGGNYNYLYRLFRKSN

SEQ ID NO: 18 - protein, 20 aa, Unknown
  SARS-CoV sequence
LWLLWPVTLACFVLAAVYRI

SEQ ID NO: 19 - protein, 21 aa, Unknown
  SARS-CoV sequence
LFARTRSMWSFNPETNILLNV

SEQ ID NO: 20 - protein, 23 aa, Unknown
  SARS-CoV sequence
IKDLPKEITVATSRTLSYYKLGA

SEQ ID NO: 21 - protein, 25 aa, Unknown
  SARS-CoV sequence
KQRRPQGLPNNTASWFTALTQHGKE

SEQ ID NO: 22 - protein, 26 aa, Unknown
  SARS-CoV sequence
GANKDGIIWVATEGALNTPKDHIGTR

SEQ ID NO: 23 - protein, 35 aa, Unknown
  SARS-CoV sequence
TGPEAGLPYGANKDGIIWVATEGALNTPKDHIGTR

SEQ ID NO: 24 - protein, 33 aa, Unknown
  SARS-CoV sequence
VLQLPQGTTLPKGFYAEGSRGGSQASSRSSSRS

SEQ ID NO: 25 - protein, 29 aa, Unknown
  SARS-CoV sequence
VLQLPQGTTLPKGFYAEGSRGGSQASSRS

SEQ ID NO: 26 - protein, 23 aa, Unknown
  SARS-CoV sequence
GDAALALLLLDRLNQLESKMSGK

SEQ ID NO: 27 - protein, 24 aa, Unknown
  SARS-CoV sequence
GMSRIGMEVTPSGTWLTYTGAIKL

SEQ ID NO: 28 - protein, 22 aa, Unknown
  SARS-CoV sequence
AFFGMSRIGMEVTPSGTWLTYT

SEQ ID NO: 29 - protein, 27 aa, Unknown
  SARS-CoV sequence
SKVGGNYNYLYRLFRKSNLKPFERDIS

SEQ ID NO: 30 - protein, 26 aa, Unknown
  SARS-CoV sequence
HSTQDLFLPFFSNVTWFHAIHVSGTN

SEQ ID NO: 31 - protein, 26 aa, Unknown
  SARS-CoV sequence
GINITRFQTLLALHRSYLTPGDSSSG

SEQ ID NO: 32 - protein, 33 aa, Unknown
  SARS-CoV sequence
PLVDLPIGINITRFQTLLALHRSYLTPGDSSSG

SEQ ID NO: 33 - protein, 28 aa, Unknown
  SARS-CoV sequence
VEKGIYQTSNFRVQPTESIVRFPNITNL

SEQ ID NO: 34 - protein, 25 aa, Unknown
  SARS-CoV sequence
ASQSIIAYTMSLGAENSVAYSNNSI

SEQ ID NO: 35 - protein, 26 aa, Unknown
  SARS-CoV sequence
LDSKTQSLLIVNNATNVVIKVCEFQF

SEQ ID NO: 36 - protein, 33 aa, Unknown
  SARS-CoV sequence
LPNNTASWFTALTQHGKEDLKFPRGQGVPINTN

SEQ ID NO: 37 - protein, 28 aa, Unknown
  SARS-CoV sequence
SSPDDQIGYYRRATRRIRGGDGKMKDLS

SEQ ID NO: 38 - protein, 28 aa, Unknown
  SARS-CoV sequence
KMKDLSPRWYFYYLGTGPEAGLPYGANK

SEQ ID NO: 39 - protein, 28 aa, Unknown
  SARS-CoV sequence
GNGGDAALALLLLDRLNQLESKMSGKGQ

SEQ ID NO: 40 - protein, 27 aa, Unknown
  SARS-CoV sequence
APSASAFFGMSRIGMEVTPSGTWLTYT

SEQ ID NO: 41 - protein, 31 aa, Unknown
  SARS-CoV sequence
PNFKDQVILLNKHIDAYKTFPPTEPKKDKKK

SEQ ID NO: 42 - protein, 25 aa, Unknown
  SARS-CoV sequence
VILLNKHIDAYKTFPPTEPKKDKKK

SEQ ID NO: 43 - protein, 21 aa, Unknown
  SARS-CoV sequence
NKHIDAYKTFPPTEPKKDKKK

SEQ ID NO: 44 - protein, 22 aa, Unknown
  SARS-CoV sequence
YNVTQAFGRRGPEQTQGNFGDQ

SEQ ID NO: 45 - protein, 24 aa, Unknown
  SARS-CoV sequence
IRQGTDYKHWPQIAQFAPSASAFF

SEQ ID NO: 46 - protein, 1273 aa, Unknown
  SARS-CoV sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - protein, 419 aa, Unknown
  SARS-CoV sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 48 - protein, 222 aa, Unknown
  SARS-CoV sequence
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 49 - protein, 1255 aa, Unknown
  SARS-CoV sequence
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 50 - protein, 422 aa, Unknown
  SARS-CoV sequence
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 51 - protein, 221 aa, Unknown
  SARS-CoV sequence
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 52 - protein, 9 aa, Unknown
  SARS-CoV sequence
ALNTLVKQL

SEQ ID NO: 53 - protein, 9 aa, Unknown
  SARS-CoV sequence
VLNDILSRL

SEQ ID NO: 54 - protein, 9 aa, Unknown
  SARS-CoV sequence
LITGRLQSL

SEQ ID NO: 55 - protein, 18 aa, Unknown
  SARS-CoV sequence
QLIRAAEIRASANLAATK

SEQ ID NO: 56 - protein, 9 aa, Unknown
  SARS-CoV sequence
RLNEVAKNL

SEQ ID NO: 57 - protein, 9 aa, Unknown
  SARS-CoV sequence
FIAGLIAIV

SEQ ID NO: 58 - protein, 15 aa, Unknown
  SARS-CoV sequence
AYRFNGIGVTQNVLY

SEQ ID NO: 59 - protein, 11 aa, Unknown
  SARS-CoV sequence
FLPFFSNVTWF

SEQ ID NO: 60 - protein, 9 aa, Unknown
  SARS-CoV sequence
LLALHRSYL

SEQ ID NO: 61 - protein, 9 aa, Unknown
  SARS-CoV sequence
VRFPNITNL

SEQ ID NO: 62 - protein, 10 aa, Unknown
  SARS-CoV sequence
IYQTSNFRVQ

SEQ ID NO: 63 - protein, 10 aa, Unknown
  SARS-CoV sequence
SIIAYTMSLG

SEQ ID NO: 64 - protein, 11 aa, Unknown
  SARS-CoV sequence
IVNNATNVVIK

SEQ ID NO: 65 - protein, 8 aa, Unknown
  SARS-CoV sequence
LPNNTASW

SEQ ID NO: 66 - protein, 9 aa, Unknown
  SARS-CoV sequence
ALNTPKDHI

SEQ ID NO: 67 - protein, 9 aa, Unknown
  SARS-CoV sequence
LQLPQGTTL

SEQ ID NO: 68 - protein, 12 aa, Unknown
  SARS-CoV sequence
LALLLLDRLNQL

SEQ ID NO: 69 - protein, 9 aa, Unknown
  SARS-CoV sequence
GMSRIGMEV

SEQ ID NO: 70 - protein, 9 aa, Unknown
  SARS-CoV sequence
FTALTQHGK

SEQ ID NO: 71 - protein, 9 aa, Unknown
  SARS-CoV sequence
FPRGQGVPI

SEQ ID NO: 72 - protein, 11 aa, Unknown
  SARS-CoV sequence
YYRRATRRIRG

SEQ ID NO: 73 - protein, 9 aa, Unknown
  SARS-CoV sequence
LSPRWYFYY

SEQ ID NO: 74 - protein, 12 aa, Unknown
  SARS-CoV sequence
LALLLLDRLNQL

SEQ ID NO: 75 - protein, 9 aa, Unknown
  SARS-CoV sequence
GMSRIGMEV

SEQ ID NO: 76 - protein, 8 aa, Unknown
  SARS-CoV sequence
ILLNKHID

SEQ ID NO: 77 - protein, 9 aa, Unknown
  SARS-CoV sequence
KTFPPTEPK

SEQ ID NO: 78 - protein, 11 aa, Unknown
  SARS-CoV sequence
NVTQAFGRRGP

SEQ ID NO: 79 - protein, 10 aa, Unknown
  SARS-CoV sequence
KHWPQIAQFA

SEQ ID NO: 80 - protein, 10 aa, Unknown
  SARS-CoV sequence
TLACFVLAAV

SEQ ID NO: 81 - protein, 9 aa, Unknown
  SARS-CoV sequence
SFNPETNIL

SEQ ID NO: 82 - protein, 10 aa, Unknown
  SARS-CoV sequence
TVATSRTLSY

SEQ ID NO: 83 - protein, 11 aa, Unknown
  SARS-CoV sequence
IAWNSNNLDSK

SEQ ID NO: 84 - protein, 15 aa, Unknown
  SARS-CoV sequence
SKVGGNYNYLYRLFR

SEQ ID NO: 85 - protein, 9 aa, Unknown
  SARS-CoV sequence
CNGVEGFNC

SEQ ID NO: 86 - protein, 19 aa, Unknown
  SARS-CoV sequence
NCYFPLQSYGFQPTNGVGY

SEQ ID NO: 87 - protein, 15 aa, Unknown
  SARS-CoV sequence
GNYNYLYRLFRKSNL

SEQ ID NO: 88 - protein, 12 aa, Unknown
  SARS-CoV sequence
ALNTLVKQLSSN

SEQ ID NO: 89 - protein, 12 aa, Unknown
  SARS-CoV sequence
VLNDILSRLDKV

SEQ ID NO: 90 - protein, 12 aa, Unknown
  SARS-CoV sequence
LITGRLQSLQTY

SEQ ID NO: 91 - protein, 18 aa, Unknown
  SARS-CoV sequence
QLIRAAEIRASANLAATK

SEQ ID NO: 92 - protein, 12 aa, Unknown
  SARS-CoV sequence
RLNEVAKNLNES

SEQ ID NO: 93 - protein, 12 aa, Unknown
  SARS-CoV sequence
FIAGLIAIVMVT

SEQ ID NO: 94 - protein, 15 aa, Unknown
  SARS-CoV sequence
AYRFNGIGVTQNVLY

SEQ ID NO: 95 - protein, 14 aa, Unknown
  SARS-CoV sequence
FLPFFSNVTWFHAI

SEQ ID NO: 96 - protein, 12 aa, Unknown
  SARS-CoV sequence
LLALHRSYLTPG

SEQ ID NO: 97 - protein, 12 aa, Unknown
  SARS-CoV sequence
VRFPNITNLCPF

SEQ ID NO: 98 - protein, 13 aa, Unknown
  SARS-CoV sequence
IYQTSNFRVQPTE

SEQ ID NO: 99 - protein, 13 aa, Unknown
  SARS-CoV sequence
SIIAYTMSLGAEN

SEQ ID NO: 100 - protein, 14 aa, Unknown
  SARS-CoV sequence
IVNNATNVVIKVCE

SEQ ID NO: 101 - protein, 11 aa, Unknown
  SARS-CoV sequence
LPNNTASWFTA

SEQ ID NO: 102 - protein, 12 aa, Unknown
  SARS-CoV sequence
ALNTPKDHIGTR

SEQ ID NO: 103 - protein, 12 aa, Unknown
  SARS-CoV sequence
LQLPQGTTLPKG

SEQ ID NO: 104 - protein, 15 aa, Unknown
  SARS-CoV sequence
LALLLLDRLNQLESK

SEQ ID NO: 105 - protein, 19 aa, Unknown
  SARS-CoV sequence
GMSRIGMEVTPSGTWLTYT

SEQ ID NO: 106 - protein, 12 aa, Unknown
  SARS-CoV sequence
FTALTQHGKEDL

SEQ ID NO: 107 - protein, 12 aa, Unknown
  SARS-CoV sequence
FPRGQGVPINTN

SEQ ID NO: 108 - protein, 14 aa, Unknown
  SARS-CoV sequence
YYRRATRRIRGGDG

SEQ ID NO: 109 - protein, 12 aa, Unknown
  SARS-CoV sequence
LSPRWYFYYLGT

SEQ ID NO: 110 - protein, 15 aa, Unknown
  SARS-CoV sequence
LALLLLDRLNQLESK

SEQ ID NO: 111 - protein, 12 aa, Unknown
  SARS-CoV sequence
GMSRIGMEVTPS

SEQ ID NO: 112 - protein, 11 aa, Unknown
  SARS-CoV sequence
ILLNKHIDAYK

SEQ ID NO: 113 - protein, 12 aa, Unknown
  SARS-CoV sequence
KTFPPTEPKKDK

SEQ ID NO: 114 - protein, 14 aa, Unknown
  SARS-CoV sequence
NVTQAFGRRGPEQT

SEQ ID NO: 115 - protein, 13 aa, Unknown
  SARS-CoV sequence
KHWPQIAQFAPSA

SEQ ID NO: 116 - protein, 13 aa, Unknown
  SARS-CoV sequence
TLACFVLAAVYRI

SEQ ID NO: 117 - protein, 12 aa, Unknown
  SARS-CoV sequence
SFNPETNILLNV

SEQ ID NO: 118 - protein, 13 aa, Unknown
  SARS-CoV sequence
TVATSRTLSYYKL

SEQ ID NO: 119 - protein, 14 aa, Unknown
  SARS-CoV sequence
IAWNSNNLDSKVGG

SEQ ID NO: 120 - protein, 15 aa, Unknown
  SARS-CoV sequence
SKVGGNYNYLYRLFR

SEQ ID NO: 121 - protein, 9 aa, Unknown
  SARS-CoV sequence
CNGVEGFNC

SEQ ID NO: 122 - protein, 19 aa, Unknown
  SARS-CoV sequence
NCYFPLQSYGFQPTNGVGY

SEQ ID NO: 123 - protein, 15 aa, Unknown
  SARS-CoV sequence
GNYNYLYRLFRKSNL

SEQ ID NO: 124 - protein, 10 aa, Unknown
  SARS-CoV sequence
SVLNDILSRL

SEQ ID NO: 125 - protein, 9 aa, Unknown
  SARS-CoV sequence
SVLNDILSR

SEQ ID NO: 126 - protein, 11 aa, Unknown
  SARS-CoV sequence
ITGRLQSLQTY

SEQ ID NO: 127 - protein, 11 aa, Unknown
  SARS-CoV sequence
RLITGRLQSLQ

SEQ ID NO: 128 - protein, 10 aa, Unknown
  SARS-CoV sequence
LITGRLQSLQ

SEQ ID NO: 129 - protein, 9 aa, Unknown
  SARS-CoV sequence
GRLQSLQTY

SEQ ID NO: 130 - protein, 11 aa, Unknown
  SARS-CoV sequence
RLNEVAKNLNE

SEQ ID NO: 131 - protein, 9 aa, Unknown
  SARS-CoV sequence
NLNESLIDL

SEQ ID NO: 132 - protein, 9 aa, Unknown
  SARS-CoV sequence
KEIDRLNEV

SEQ ID NO: 133 - protein, 11 aa, Unknown
  SARS-CoV sequence
EVAKNLNESLI

SEQ ID NO: 134 - protein, 8 aa, Unknown
  SARS-CoV sequence
GLIAIVMV

SEQ ID NO: 135 - protein, 9 aa, Unknown
  SARS-CoV sequence
QYIKWPWYI

SEQ ID NO: 136 - protein, 10 aa, Unknown
  SARS-CoV sequence
QYIKWPWYIW

SEQ ID NO: 137 - protein, 10 aa, Unknown
  SARS-CoV sequence
KWPWYIWLGF

SEQ ID NO: 138 - protein, 11 aa, Unknown
  SARS-CoV sequence
QYIKWPWYIWL

SEQ ID NO: 139 - protein, 8 aa, Unknown
  SARS-CoV sequence
KYEQYIKW

SEQ ID NO: 140 - protein, 8 aa, Unknown
  SARS-CoV sequence
KWPWYIWL

SEQ ID NO: 141 - protein, 9 aa, Unknown
  SARS-CoV sequence
IWLGFIAGL

SEQ ID NO: 142 - protein, 9 aa, Unknown
  SARS-CoV sequence
YIKWPWYIW

SEQ ID NO: 143 - protein, 11 aa, Unknown
  SARS-CoV sequence
WPWYIWLGFIA

SEQ ID NO: 144 - protein, 9 aa, Unknown
  SARS-CoV sequence
YEQYIKWPW

SEQ ID NO: 145 - protein, 9 aa, Unknown
  SARS-CoV sequence
LQIPFAMQM

SEQ ID NO: 146 - protein, 11 aa, Unknown
  SARS-CoV sequence
FAMQMAYRFNG

SEQ ID NO: 147 - protein, 10 aa, Unknown
  SARS-CoV sequence
QIPFAMQMAY

SEQ ID NO: 148 - protein, 9 aa, Unknown
  SARS-CoV sequence
IPFAMQMAY

SEQ ID NO: 149 - protein, 11 aa, Unknown
  SARS-CoV sequence
LQIPFAMQMAY

SEQ ID NO: 150 - protein, 11 aa, Unknown
  SARS-CoV sequence
IPFAMQMAYRF

SEQ ID NO: 151 - protein, 9 aa, Unknown
  SARS-CoV sequence
VTWFHAIHV

SEQ ID NO: 152 - protein, 11 aa, Unknown
  SARS-CoV sequence
LFLPFFSNVTW

SEQ ID NO: 153 - protein, 11 aa, Unknown
  SARS-CoV sequence
STQDLFLPFFS

SEQ ID NO: 154 - protein, 11 aa, Unknown
  SARS-CoV sequence
LPFFSNVTWFH

SEQ ID NO: 155 - protein, 11 aa, Unknown
  SARS-CoV sequence
NVTWFHAIHVS

SEQ ID NO: 156 - protein, 10 aa, Unknown
  SARS-CoV sequence
NVTWFHAIHV

SEQ ID NO: 157 - protein, 8 aa, Unknown
  SARS-CoV sequence
LPFFSNVT

SEQ ID NO: 158 - protein, 8 aa, Unknown
  SARS-CoV sequence
FLPFFSNV

SEQ ID NO: 159 - protein, 11 aa, Unknown
  SARS-CoV sequence
TLLALHRSYLT

SEQ ID NO: 160 - protein, 11 aa, Unknown
  SARS-CoV sequence
NITRFQTLLAL

SEQ ID NO: 161 - protein, 10 aa, Unknown
  SARS-CoV sequence
TLLALHRSYL

SEQ ID NO: 162 - protein, 11 aa, Unknown
  SARS-CoV sequence
LPIGINITRFQ

SEQ ID NO: 163 - protein, 10 aa, Unknown
  SARS-CoV sequence
INITRFQTLL

SEQ ID NO: 164 - protein, 10 aa, Unknown
  SARS-CoV sequence
NITRFQTLLA

SEQ ID NO: 165 - protein, 8 aa, Unknown
  SARS-CoV sequence
LPIGINIT

SEQ ID NO: 166 - protein, 10 aa, Unknown
  SARS-CoV sequence
IYQTSNFRVQ

SEQ ID NO: 167 - protein, 11 aa, Unknown
  SARS-CoV sequence
KGIYQTSNFRV

SEQ ID NO: 168 - protein, 11 aa, Unknown
  SARS-CoV sequence
QPTESIVRFPN

SEQ ID NO: 169 - protein, 11 aa, Unknown
  SARS-CoV sequence
KGIYQTSNFRV

SEQ ID NO: 170 - protein, 11 aa, Unknown
  SARS-CoV sequence
QPTESIVRFPN

SEQ ID NO: 171 - protein, 11 aa, Unknown
  SARS-CoV sequence
SQSIIAYTMSL

SEQ ID NO: 172 - protein, 9 aa, Unknown
  SARS-CoV sequence
SVAYSNNSI

SEQ ID NO: 173 - protein, 11 aa, Unknown
  SARS-CoV sequence
LGAENSVAYSN

SEQ ID NO: 174 - protein, 9 aa, Unknown
  SARS-CoV sequence
VIKVCEFQF

SEQ ID NO: 175 - protein, 11 aa, Unknown
  SARS-CoV sequence
NATNVVIKVCE

SEQ ID NO: 176 - protein, 9 aa, Unknown
  SARS-CoV sequence
NVVIKVCEF

SEQ ID NO: 177 - protein, 9 aa, Unknown
  SARS-CoV sequence
FTALTQHGK

SEQ ID NO: 178 - protein, 11 aa, Unknown
  SARS-CoV sequence
NTASWFTALTQ

SEQ ID NO: 179 - protein, 11 aa, Unknown
  SARS-CoV sequence
GLPNNTASWFT

SEQ ID NO: 180 - protein, 10 aa, Unknown
  SARS-CoV sequence
LPNNTASWFT

SEQ ID NO: 181 - protein, 9 aa, Unknown
  SARS-CoV sequence
IIWVATEGA

SEQ ID NO: 182 - protein, 10 aa, Unknown
  SARS-CoV sequence
ALNTPKDHIG

SEQ ID NO: 183 - protein, 9 aa, Unknown
  SARS-CoV sequence
GTTLPKGFY

SEQ ID NO: 184 - protein, 11 aa, Unknown
  SARS-CoV sequence
LQLPQGTTLPK

SEQ ID NO: 185 - protein, 9 aa, Unknown
  SARS-CoV sequence
TTLPKGFYA

SEQ ID NO: 186 - protein, 11 aa, Unknown
  SARS-CoV sequence
ALALLLLDRLN

SEQ ID NO: 187 - protein, 11 aa, Unknown
  SARS-CoV sequence
RLNQLESKMSG

SEQ ID NO: 188 - protein, 11 aa, Unknown
  SARS-CoV sequence
VTPSGTWLTYT

SEQ ID NO: 189 - protein, 9 aa, Unknown
  SARS-CoV sequence
TWLTYTGAI

SEQ ID NO: 190 - protein, 10 aa, Unknown
  SARS-CoV sequence
EVTPSGTWLT

SEQ ID NO: 191 - protein, 9 aa, Unknown
  SARS-CoV sequence
TPSGTWLTY

SEQ ID NO: 192 - protein, 11 aa, Unknown
  SARS-CoV sequence
NTASWFTALTQ

SEQ ID NO: 193 - protein, 9 aa, Unknown
  SARS-CoV sequence
FPRGQGVPI

SEQ ID NO: 194 - protein, 8 aa, Unknown
  SARS-CoV sequence
LPNNTASW

SEQ ID NO: 195 - protein, 11 aa, Unknown
  SARS-CoV sequence
NTASWFTALTQ

SEQ ID NO: 196 - protein, 9 aa, Unknown
  SARS-CoV sequence
FTALTQHGK

SEQ ID NO: 197 - protein, 8 aa, Unknown
  SARS-CoV sequence
LPNNTASW

SEQ ID NO: 198 - protein, 8 aa, Unknown
  SARS-CoV sequence
SSPDDQIG

SEQ ID NO: 199 - protein, 9 aa, Unknown
  SARS-CoV sequence
IGYYRRATR

SEQ ID NO: 200 - protein, 11 aa, Unknown
  SARS-CoV sequence
GYYRRATRRIR

SEQ ID NO: 201 - protein, 9 aa, Unknown
  SARS-CoV sequence
SPDDQIGYY

SEQ ID NO: 202 - protein, 9 aa, Unknown
  SARS-CoV sequence
DLSPRWYFY

SEQ ID NO: 203 - protein, 10 aa, Unknown
  SARS-CoV sequence
DLSPRWYFYY

SEQ ID NO: 204 - protein, 8 aa, Unknown
  SARS-CoV sequence
GTGPEAGL

SEQ ID NO: 205 - protein, 11 aa, Unknown
  SARS-CoV sequence
MKDLSPRWYFY

SEQ ID NO: 206 - protein, 10 aa, Unknown
  SARS-CoV sequence
KDLSPRWYFY

SEQ ID NO: 207 - protein, 10 aa, Unknown
  SARS-CoV sequence
LSPRWYFYYL

SEQ ID NO: 208 - protein, 8 aa, Unknown
  SARS-CoV sequence
YLGTGPEA

SEQ ID NO: 209 - protein, 9 aa, Unknown
  SARS-CoV sequence
KMKDLSPRW

SEQ ID NO: 210 - protein, 11 aa, Unknown
  SARS-CoV sequence
KDLSPRWYFYY

SEQ ID NO: 211 - protein, 10 aa, Unknown
  SARS-CoV sequence
KMKDLSPRWY

SEQ ID NO: 212 - protein, 11 aa, Unknown
  SARS-CoV sequence
KMKDLSPRWYF

SEQ ID NO: 213 - protein, 9 aa, Unknown
  SARS-CoV sequence
KDLSPRWYF

SEQ ID NO: 214 - protein, 11 aa, Unknown
  SARS-CoV sequence
SPRWYFYYLGT

SEQ ID NO: 215 - protein, 11 aa, Unknown
  SARS-CoV sequence
ALALLLLDRLN

SEQ ID NO: 216 - protein, 11 aa, Unknown
  SARS-CoV sequence
RLNQLESKMSG

SEQ ID NO: 217 - protein, 11 aa, Unknown
  SARS-CoV sequence
VTPSGTWLTYT

SEQ ID NO: 218 - protein, 9 aa, Unknown
  SARS-CoV sequence
EVTPSGTWL

SEQ ID NO: 219 - protein, 11 aa, Unknown
  SARS-CoV sequence
GMSRIGMEVTP

SEQ ID NO: 220 - protein, 10 aa, Unknown
  SARS-CoV sequence
EVTPSGTWLT

SEQ ID NO: 221 - protein, 9 aa, Unknown
  SARS-CoV sequence
TPSGTWLTY

SEQ ID NO: 222 - protein, 10 aa, Unknown
  SARS-CoV sequence
MEVTPSGTWL

SEQ ID NO: 223 - protein, 9 aa, Unknown
  SARS-CoV sequence
LLNKHIDAY

SEQ ID NO: 224 - protein, 11 aa, Unknown
  SARS-CoV sequence
KTFPPTEPKKD

SEQ ID NO: 225 - protein, 10 aa, Unknown
  SARS-CoV sequence
LLNKHIDAYK

SEQ ID NO: 226 - protein, 8 aa, Unknown
  SARS-CoV sequence
KTFPPTEP

SEQ ID NO: 227 - protein, 9 aa, Unknown
  SARS-CoV sequence
KHIDAYKTF

SEQ ID NO: 228 - protein, 11 aa, Unknown
  SARS-CoV sequence
VILLNKHIDAY

SEQ ID NO: 229 - protein, 9 aa, Unknown
  SARS-CoV sequence
LLNKHIDAY

SEQ ID NO: 230 - protein, 11 aa, Unknown
  SARS-CoV sequence
KTFPPTEPKKD

SEQ ID NO: 231 - protein, 10 aa, Unknown
  SARS-CoV sequence
LLNKHIDAYK

SEQ ID NO: 232 - protein, 9 aa, Unknown
  SARS-CoV sequence
KHIDAYKTF

SEQ ID NO: 233 - protein, 11 aa, Unknown
  SARS-CoV sequence
VILLNKHIDAY

SEQ ID NO: 234 - protein, 8 aa, Unknown
  SARS-CoV sequence
ILLNKHID

SEQ ID NO: 235 - protein, 8 aa, Unknown
  SARS-CoV sequence
QFAPSASA

SEQ ID NO: 236 - protein, 9 aa, Unknown
  SARS-CoV sequence
RQGTDYKHW

SEQ ID NO: 237 - protein, 8 aa, Unknown
  SARS-CoV sequence
TLACFVLA

SEQ ID NO: 238 - protein, 9 aa, Unknown
  SARS-CoV sequence
WLLWPVTLA

SEQ ID NO: 239 - protein, 9 aa, Unknown
  SARS-CoV sequence
FVLAAVYRI

SEQ ID NO: 240 - protein, 9 aa, Unknown
  SARS-CoV sequence
LWLLWPVTL

SEQ ID NO: 241 - protein, 9 aa, Unknown
  SARS-CoV sequence
LWPVTLACF

SEQ ID NO: 242 - protein, 11 aa, Unknown
  SARS-CoV sequence
ACFVLAAVYRI

SEQ ID NO: 243 - protein, 10 aa, Unknown
  SARS-CoV sequence
WPVTLACFVL

SEQ ID NO: 244 - protein, 10 aa, Unknown
  SARS-CoV sequence
SMWSFNPETN

SEQ ID NO: 245 - protein, 9 aa, Unknown
  SARS-CoV sequence
FNPETNILL

SEQ ID NO: 246 - protein, 9 aa, Unknown
  SARS-CoV sequence
LFARTRSMW

SEQ ID NO: 247 - protein, 11 aa, Unknown
  SARS-CoV sequence
LFARTRSMWSF

SEQ ID NO: 248 - protein, 10 aa, Unknown
  SARS-CoV sequence
RTRSMWSFNP

SEQ ID NO: 249 - protein, 11 aa, Unknown
  SARS-CoV sequence
ARTRSMWSFNP

SEQ ID NO: 250 - protein, 8 aa, Unknown
  SARS-CoV sequence
FARTRSMW

SEQ ID NO: 251 - protein, 10 aa, Unknown
  SARS-CoV sequence
FARTRSMWSF

SEQ ID NO: 252 - protein, 10 aa, Unknown
  SARS-CoV sequence
VATSRTLSYY

SEQ ID NO: 253 - protein, 11 aa, Unknown
  SARS-CoV sequence
ATSRTLSYYKL

SEQ ID NO: 254 - protein, 11 aa, Unknown
  SARS-CoV sequence
ITVATSRTLSY

SEQ ID NO: 255 - protein, 9 aa, Unknown
  SARS-CoV sequence
VATSRTLSY

SEQ ID NO: 256 - protein, 11 aa, Unknown
  SARS-CoV sequence
TVATSRTLSYY

SEQ ID NO: 257 - protein, 10 aa, Unknown
  SARS-CoV sequence
ATSRTLSYYK

SEQ ID NO: 258 - protein, 8 aa, Unknown
  SARS-CoV sequence
ATSRTLSY

SEQ ID NO: 259 - protein, 11 aa, Unknown
  SARS-CoV sequence
VATSRTLSYYK

SEQ ID NO: 260 - protein, 11 aa, Unknown
  SARS-CoV sequence
TSRTLSYYKLG

SEQ ID NO: 261 - protein, 11 aa, Unknown
  SARS-CoV sequence
SRTLSYYKLGA

SEQ ID NO: 262 - protein, 9 aa, Unknown
  SARS-CoV sequence
ITVATSRTL

SEQ ID NO: 263 - protein, 11 aa, Unknown
  SARS-CoV sequence
LPKEITVATSR

SEQ ID NO: 264 - protein, 15 aa, Unknown
  SARS-CoV sequence
GCVIAWNSNNLDSKV

SEQ ID NO: 265 - protein, 15 aa, Unknown
  SARS-CoV sequence
KVGGNYNYLYRLFRK

SEQ ID NO: 266 - protein, 15 aa, Unknown
  SARS-CoV sequence
VGGNYNYLYRLFRKS

SEQ ID NO: 267 - protein, 15 aa, Unknown
  SARS-CoV sequence
GGNYNYLYRLFRKSN

SEQ ID NO: 268 - protein, 10 aa, Unknown
  SARS-CoV sequence
KVGGNYNYLY

SEQ ID NO: 269 - protein, 9 aa, Unknown
  SARS-CoV sequence
VIAWNSNNL

SEQ ID NO: 270 - protein, 9 aa, Unknown
  SARS-CoV sequence
KVGGNYNYL

SEQ ID NO: 271 - protein, 9 aa, Unknown
  SARS-CoV sequence
NYNYLYRLF

SEQ ID NO: 272 - protein, 11 aa, Unknown
  SARS-CoV sequence
SKVGGNYNYLY

SEQ ID NO: 273 - protein, 9 aa, Unknown
  SARS-CoV sequence
VGGNYNYLY

SEQ ID NO: 274 - protein, 10 aa, Unknown
  SARS-CoV sequence
NYNYLYRLFR

SEQ ID NO: 275 - protein, 9 aa, Unknown
  SARS-CoV sequence
YNYLYRLFR

SEQ ID NO: 276 - protein, 9 aa, Unknown
  SARS-CoV sequence
YLYRLFRKS

SEQ ID NO: 277 - protein, 15 aa, Unknown
  SARS-CoV sequence
KVGGNYNYLYRLFRK

SEQ ID NO: 278 - protein, 15 aa, Unknown
  SARS-CoV sequence
VGGNYNYLYRLFRKS

SEQ ID NO: 279 - protein, 15 aa, Unknown
  SARS-CoV sequence
GGNYNYLYRLFRKSN

SEQ ID NO: 280 - protein, 15 aa, Unknown
  SARS-CoV sequence
GNYNYLYRLFRKSNL

SEQ ID NO: 281 - protein, 15 aa, Unknown
  SARS-CoV sequence
NYNYLYRLFRKSNLK

SEQ ID NO: 282 - protein, 15 aa, Unknown
  SARS-CoV sequence
GCVIAWNSNNLDSKV

SEQ ID NO: 283 - protein, 10 aa, Unknown
  SARS-CoV sequence
KVGGNYNYLY

SEQ ID NO: 284 - protein, 9 aa, Unknown
  SARS-CoV sequence
KVGGNYNYL

SEQ ID NO: 285 - protein, 9 aa, Unknown
  SARS-CoV sequence
NYNYLYRLF

SEQ ID NO: 286 - protein, 11 aa, Unknown
  SARS-CoV sequence
SKVGGNYNYLY

SEQ ID NO: 287 - protein, 9 aa, Unknown
  SARS-CoV sequence
VGGNYNYLY

SEQ ID NO: 288 - protein, 10 aa, Unknown
  SARS-CoV sequence
NYNYLYRLFR

SEQ ID NO: 289 - protein, 9 aa, Unknown
  SARS-CoV sequence
YNYLYRLFR

SEQ ID NO: 290 - protein, 9 aa, Unknown
  SARS-CoV sequence
YLYRLFRKS

SEQ ID NO: 291 - protein, 9 aa, Unknown
  SARS-CoV sequence
VIAWNSNNL

SEQ ID NO: 292 - protein, 15 aa, Unknown
  SARS-CoV sequence
NGVEGFNCYFPLQSY

SEQ ID NO: 293 - protein, 15 aa, Unknown
  SARS-CoV sequence
GVEGFNCYFPLQSYG

SEQ ID NO: 294 - protein, 15 aa, Unknown
  SARS-CoV sequence
VEGFNCYFPLQSYGF

SEQ ID NO: 295 - protein, 15 aa, Unknown
  SARS-CoV sequence
EGFNCYFPLQSYGFQ

SEQ ID NO: 296 - protein, 15 aa, Unknown
  SARS-CoV sequence
GFNCYFPLQSYGFQP

SEQ ID NO: 297 - protein, 8 aa, Unknown
  SARS-CoV sequence
CYFPLQSY

SEQ ID NO: 298 - protein, 11 aa, Unknown
  SARS-CoV sequence
YFPLQSYGFQP

SEQ ID NO: 299 - protein, 11 aa, Unknown
  SARS-CoV sequence
NCYFPLQSYGF

SEQ ID NO: 300 - protein, 10 aa, Unknown
  SARS-CoV sequence
FPLQSYGFQP

SEQ ID NO: 301 - protein, 15 aa, Unknown
  SARS-CoV sequence
NGVEGFNCYFPLQSY

SEQ ID NO: 302 - protein, 15 aa, Unknown
  SARS-CoV sequence
GVEGFNCYFPLQSYG

SEQ ID NO: 303 - protein, 15 aa, Unknown
  SARS-CoV sequence
VEGFNCYFPLQSYGF

SEQ ID NO: 304 - protein, 15 aa, Unknown
  SARS-CoV sequence
EGFNCYFPLQSYGFQ

SEQ ID NO: 305 - protein, 15 aa, Unknown
  SARS-CoV sequence
GFNCYFPLQSYGFQP

SEQ ID NO: 306 - protein, 15 aa, Unknown
  SARS-CoV sequence
KVGGNYNYLYRLFRK

SEQ ID NO: 307 - protein, 15 aa, Unknown
  SARS-CoV sequence
VGGNYNYLYRLFRKS

SEQ ID NO: 308 - protein, 15 aa, Unknown
  SARS-CoV sequence
GGNYNYLYRLFRKSN

SEQ ID NO: 309 - protein, 15 aa, Unknown
  SARS-CoV sequence
GNYNYLYRLFRKSNL

SEQ ID NO: 310 - protein, 15 aa, Unknown
  SARS-CoV sequence
NYNYLYRLFRKSNLK

SEQ ID NO: 311 - protein, 15 aa, Unknown
  SARS-CoV sequence
YNYLYRLFRKSNLKP

SEQ ID NO: 312 - protein, 15 aa, Unknown
  SARS-CoV sequence
NYLYRLFRKSNLKPF

SEQ ID NO: 313 - protein, 15 aa, Unknown
  SARS-CoV sequence
YLYRLFRKSNLKPFE

SEQ ID NO: 314 - protein, 15 aa, Unknown
  SARS-CoV sequence
LYRLFRKSNLKPFER

SEQ ID NO: 315 - protein, 15 aa, Unknown
  SARS-CoV sequence
YRLFRKSNLKPFERD

SEQ ID NO: 316 - protein, 10 aa, Unknown
  SARS-CoV sequence
KVGGNYNYLY

SEQ ID NO: 317 - protein, 9 aa, Unknown
  SARS-CoV sequence
KVGGNYNYL

SEQ ID NO: 318 - protein, 10 aa, Unknown
  SARS-CoV sequence
RLFRKSNLKP

SEQ ID NO: 319 - protein, 11 aa, Unknown
  SARS-CoV sequence
SKVGGNYNYLY

SEQ ID NO: 320 - protein, 9 aa, Unknown
  SARS-CoV sequence
VGGNYNYLY

SEQ ID NO: 321 - protein, 9 aa, Unknown
  SARS-CoV sequence
KSNLKPFER

SEQ ID NO: 322 - protein, 11 aa, Unknown
  SARS-CoV sequence
YRLFRKSNLKP

SEQ ID NO: 323 - protein, 25 aa, Unknown
  SARS-CoV sequence
IRQGTDYKHWPQIAQFAPSASAFFG

SEQ ID NO: 324 - protein, 24 aa, Unknown
  SARS-CoV sequence
HSTQDLFLPFFSNVTWFHAISGTN

SEQ ID NO: 325 - protein, 30 aa, Unknown
  SARS-CoV sequence
PLVDLPIGINITRFQTLHISYLTPGDSSSG

SEQ ID NO: 326 - protein, 30 aa, Unknown
  SARS-CoV sequence
ALNTLVKQLSSNFGAISSVLNDILARLDKV

SEQ ID NO: 327 - protein, 32 aa, Unknown
  SARS-CoV sequence
VLNDILARLDKVEAEVQIDRLITGRLQSLQTY

SEQ ID NO: 328 - protein, 28 aa, Unknown
  SARS-CoV sequence
SSRDDQIGYYRRATRRIRGGDGKMKDLS

SEQ ID NO: 329 - protein, 28 aa, Unknown
  SARS-CoV sequence
GNGGDAALALLLLDRLNQLESKMFGKGQ

SEQ ID NO: 330 - protein, 29 aa, Unknown
  SARS-CoV sequence
STPCNGVEGFNCYFPLQSYGFQPTYGVGY

SEQ ID NO: 331 - protein, 29 aa, Unknown
  SARS-CoV sequence
STPCNGVKGFNCYFPLQSYGFQPTYGVGY

SEQ ID NO: 332 - protein, 419 aa, Unknown
  SARS-CoV sequence
MSLNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMFGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 333 - protein, 1270 aa, Unknown
  SARS-CoV sequence
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 334 - protein, 419 aa, Unknown
  SARS-CoV sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGISPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 335 - protein, 1270 aa, Unknown
  SARS-CoV sequence
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 336 - protein, 419 aa, Unknown
  SARS-CoV sequence
MSDNGPQNQRNAPRITFGGPSDSTGSNQNGERSGARSKQRRPQGLPNNTASWFTALTQHGKEDLKFPRGQGVPINTNSSRDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 337 - protein, 1273 aa, Unknown
  SARS-CoV sequence
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 338 - protein, 419 aa, Unknown
  SARS-CoV sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSMGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKAYETQALPQRQKKQQTVTLLPAADLDDFSKQLQQSMSSADSTQA

SEQ ID NO: 339 - protein, 1273 aa, Unknown
  SARS-CoV sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 340 - protein, 25 aa, Unknown
  SARS-CoV sequence
SNNLDSKVGGNYNYRYRLFRKSNLK

SEQ ID NO: 341 - protein, 29 aa, Unknown
  SARS-CoV sequence
STPCNGVQGFNCYFPLQSYGFQPTNGVGY

SEQ ID NO: 342 - protein, 31 aa, Unknown
  SARS-CoV sequence
TGCVIAWNSNNLDSKVGGNYNYRYRLFRKSN

SEQ ID NO: 343 - protein, 27 aa, Unknown
  SARS-CoV sequence
SKVGGNYNYRYRLFRKSNLKPFERDIS

SEQ ID NO: 344 - protein, 10 aa, Unknown
  SARS-CoV sequence
STQDLFLPFF

SEQ ID NO: 345 - protein, 11 aa, Unknown
  SARS-CoV sequence
FLPFFSNVTWF

SEQ ID NO: 346 - protein, 9 aa, Unknown
  SARS-CoV sequence
LFLPFFSNV

SEQ ID NO: 347 - protein, 10 aa, Unknown
  SARS-CoV sequence
PFFSNVTWFH

SEQ ID NO: 348 - protein, 8 aa, Unknown
  SARS-CoV sequence
FLPFFSNV

SEQ ID NO: 349 - protein, 8 aa, Unknown
  SARS-CoV sequence
FFSNVTWF

SEQ ID NO: 350 - protein, 11 aa, Unknown
  SARS-CoV sequence
LFLPFFSNVTW

SEQ ID NO: 351 - protein, 9 aa, Unknown
  SARS-CoV sequence
STQDLFLPF

SEQ ID NO: 352 - protein, 9 aa, Unknown
  SARS-CoV sequence
TQDLFLPFF

SEQ ID NO: 353 - protein, 10 aa, Unknown
  SARS-CoV sequence
LPFFSNVTWF

SEQ ID NO: 354 - protein, 9 aa, Unknown
  SARS-CoV sequence
SNVTWFHAI

SEQ ID NO: 355 - protein, 10 aa, Unknown
  SARS-CoV sequence
FFSNVTWFHA

SEQ ID NO: 356 - protein, 11 aa, Unknown
  SARS-CoV sequence
LPFFSNVTWFH

SEQ ID NO: 357 - protein, 9 aa, Unknown
  SARS-CoV sequence
FQTLHISYL

SEQ ID NO: 358 - protein, 11 aa, Unknown
  SARS-CoV sequence
RFQTLHISYLT

SEQ ID NO: 359 - protein, 10 aa, Unknown
  SARS-CoV sequence
RFQTLHISYL

SEQ ID NO: 360 - protein, 9 aa, Unknown
  SARS-CoV sequence
ITRFQTLHI

SEQ ID NO: 361 - protein, 11 aa, Unknown
  SARS-CoV sequence
INITRFQTLHI

SEQ ID NO: 362 - protein, 10 aa, Unknown
  SARS-CoV sequence
ITRFQTLHIS

SEQ ID NO: 363 - protein, 10 aa, Unknown
  SARS-CoV sequence
LPIGINITRF

SEQ ID NO: 364 - protein, 11 aa, Unknown
  SARS-CoV sequence
LPIGINITRFQ

SEQ ID NO: 365 - protein, 10 aa, Unknown
  SARS-CoV sequence
NITRFQTLHI

SEQ ID NO: 366 - protein, 8 aa, Unknown
  SARS-CoV sequence
GINITRFQ

SEQ ID NO: 367 - protein, 9 aa, Unknown
  SARS-CoV sequence
VLNDILARL

SEQ ID NO: 368 - protein, 10 aa, Unknown
  SARS-CoV sequence
SVLNDILARL

SEQ ID NO: 369 - protein, 11 aa, Unknown
  SARS-CoV sequence
LVKQLSSNFGA

SEQ ID NO: 370 - protein, 9 aa, Unknown
  SARS-CoV sequence
SVLNDILAR

SEQ ID NO: 371 - protein, 11 aa, Unknown
  SARS-CoV sequence
ITGRLQSLQTY

SEQ ID NO: 372 - protein, 9 aa, Unknown
  SARS-CoV sequence
RLDKVEAEV

SEQ ID NO: 373 - protein, 11 aa, Unknown
  SARS-CoV sequence
RLITGRLQSLQ

SEQ ID NO: 374 - protein, 9 aa, Unknown
  SARS-CoV sequence
RLQSLQTYV

SEQ ID NO: 375 - protein, 10 aa, Unknown
  SARS-CoV sequence
LITGRLQSLQ

SEQ ID NO: 376 - protein, 9 aa, Unknown
  SARS-CoV sequence
GRLQSLQTY

SEQ ID NO: 377 - protein, 8 aa, Unknown
  SARS-CoV sequence
SSRDDQIG

SEQ ID NO: 378 - protein, 11 aa, Unknown
  SARS-CoV sequence
YYRRATRRIRG

SEQ ID NO: 379 - protein, 8 aa, Unknown
  SARS-CoV sequence
GYYRRATR

SEQ ID NO: 380 - protein, 9 aa, Unknown
  SARS-CoV sequence
SSRDDQIGY

SEQ ID NO: 381 - protein, 9 aa, Unknown
  SARS-CoV sequence
IGYYRRATR

SEQ ID NO: 382 - protein, 11 aa, Unknown
  SARS-CoV sequence
GYYRRATRRIR

SEQ ID NO: 383 - protein, 11 aa, Unknown
  SARS-CoV sequence
SSRDDQIGYYR

SEQ ID NO: 384 - protein, 9 aa, Unknown
  SARS-CoV sequence
LLLDRLNQL

SEQ ID NO: 385 - protein, 11 aa, Unknown
  SARS-CoV sequence
ALALLLLDRLN

SEQ ID NO: 386 - protein, 10 aa, Unknown
  SARS-CoV sequence
LLLLDRLNQL

SEQ ID NO: 387 - protein, 11 aa, Unknown
  SARS-CoV sequence
RLNQLESKMFG

SEQ ID NO: 388 - protein, 11 aa, Unknown
  SARS-CoV sequence
DAALALLLLDR

SEQ ID NO: 389 - protein, 11 aa, Unknown
  SARS-CoV sequence
LQSYGFQPTYG

SEQ ID NO: 390 - protein, 10 aa, Unknown
  SARS-CoV sequence
QSYGFQPTYG

SEQ ID NO: 391 - protein, 9 aa, Unknown
  SARS-CoV sequence
YGFQPTYGV

SEQ ID NO: 392 - protein, 8 aa, Unknown
  SARS-CoV sequence
CYFPLQSY

SEQ ID NO: 393 - protein, 11 aa, Unknown
  SARS-CoV sequence
YFPLQSYGFQP

SEQ ID NO: 394 - protein, 8 aa, Unknown
  SARS-CoV sequence
SYGFQPTY

SEQ ID NO: 395 - protein, 10 aa, Unknown
  SARS-CoV sequence
SYGFQPTYGV

SEQ ID NO: 396 - protein, 10 aa, Unknown
  SARS-CoV sequence
GVEGFNCYFP

SEQ ID NO: 397 - protein, 11 aa, Unknown
  SARS-CoV sequence
NCYFPLQSYGF

SEQ ID NO: 398 - protein, 10 aa, Unknown
  SARS-CoV sequence
FPLQSYGFQP

SEQ ID NO: 399 - protein, 11 aa, Unknown
  SARS-CoV sequence
LQSYGFQPTYG

SEQ ID NO: 400 - protein, 10 aa, Unknown
  SARS-CoV sequence
QSYGFQPTYG

SEQ ID NO: 401 - protein, 9 aa, Unknown
  SARS-CoV sequence
YGFQPTYGV

SEQ ID NO: 402 - protein, 9 aa, Unknown
  SARS-CoV sequence
KGFNCYFPL

SEQ ID NO: 403 - protein, 8 aa, Unknown
  SARS-CoV sequence
CYFPLQSY

SEQ ID NO: 404 - protein, 11 aa, Unknown
  SARS-CoV sequence
YFPLQSYGFQP

SEQ ID NO: 405 - protein, 8 aa, Unknown
  SARS-CoV sequence
SYGFQPTY

SEQ ID NO: 406 - protein, 10 aa, Unknown
  SARS-CoV sequence
SYGFQPTYGV

SEQ ID NO: 407 - protein, 10 aa, Unknown
  SARS-CoV sequence
GVKGFNCYFP

SEQ ID NO: 408 - protein, 11 aa, Unknown
  SARS-CoV sequence
NCYFPLQSYGF

SEQ ID NO: 409 - protein, 10 aa, Unknown
  SARS-CoV sequence
FPLQSYGFQP

SEQ ID NO: 410 - protein, 11 aa, Unknown
  SARS-CoV sequence
TPCNGVKGFNC

SEQ ID NO: 411 - protein, 10 aa, Unknown
  SARS-CoV sequence
KVGGNYNYRY

SEQ ID NO: 412 - protein, 8 aa, Unknown
  SARS-CoV sequence
YRLFRKSN

SEQ ID NO: 413 - protein, 8 aa, Unknown
  SARS-CoV sequence
RYRLFRKS

SEQ ID NO: 414 - protein, 9 aa, Unknown
  SARS-CoV sequence
NYNYRYRLF

SEQ ID NO: 415 - protein, 11 aa, Unknown
  SARS-CoV sequence
DSKVGGNYNYR

SEQ ID NO: 416 - protein, 11 aa, Unknown
  SARS-CoV sequence
SKVGGNYNYRY

SEQ ID NO: 417 - protein, 9 aa, Unknown
  SARS-CoV sequence
VGGNYNYRY

SEQ ID NO: 418 - protein, 9 aa, Unknown
  SARS-CoV sequence
KVGGNYNYR

SEQ ID NO: 419 - protein, 9 aa, Unknown
  SARS-CoV sequence
YNYRYRLFR

SEQ ID NO: 420 - protein, 10 aa, Unknown
  SARS-CoV sequence
NYNYRYRLFR

SEQ ID NO: 421 - protein, 9 aa, Unknown
  SARS-CoV sequence
GNYNYRYRL

SEQ ID NO: 422 - protein, 11 aa, Unknown
  SARS-CoV sequence
NYRYRLFRKSN

SEQ ID NO: 423 - protein, 8 aa, Unknown
  SARS-CoV sequence
CYFPLQSY

SEQ ID NO: 424 - protein, 11 aa, Unknown
  SARS-CoV sequence
YFPLQSYGFQP

SEQ ID NO: 425 - protein, 10 aa, Unknown
  SARS-CoV sequence
GVQGFNCYFP

SEQ ID NO: 426 - protein, 9 aa, Unknown
  SARS-CoV sequence
QGFNCYFPL

SEQ ID NO: 427 - protein, 11 aa, Unknown
  SARS-CoV sequence
NCYFPLQSYGF

SEQ ID NO: 428 - protein, 11 aa, Unknown
  SARS-CoV sequence
TPCNGVQGFNC

SEQ ID NO: 429 - protein, 10 aa, Unknown
  SARS-CoV sequence
FPLQSYGFQP

SEQ ID NO: 430 - protein, 10 aa, Unknown
  SARS-CoV sequence
KVGGNYNYRY

SEQ ID NO: 431 - protein, 9 aa, Unknown
  SARS-CoV sequence
VIAWNSNNL

SEQ ID NO: 432 - protein, 9 aa, Unknown
  SARS-CoV sequence
NYNYRYRLF

SEQ ID NO: 433 - protein, 11 aa, Unknown
  SARS-CoV sequence
DSKVGGNYNYR

SEQ ID NO: 434 - protein, 11 aa, Unknown
  SARS-CoV sequence
SKVGGNYNYRY

SEQ ID NO: 435 - protein, 9 aa, Unknown
  SARS-CoV sequence
VGGNYNYRY

SEQ ID NO: 436 - protein, 9 aa, Unknown
  SARS-CoV sequence
KVGGNYNYR

SEQ ID NO: 437 - protein, 9 aa, Unknown
  SARS-CoV sequence
YNYRYRLFR

SEQ ID NO: 438 - protein, 10 aa, Unknown
  SARS-CoV sequence
NYNYRYRLFR

SEQ ID NO: 439 - protein, 9 aa, Unknown
  SARS-CoV sequence
GNYNYRYRL

SEQ ID NO: 440 - protein, 10 aa, Unknown
  SARS-CoV sequence
KVGGNYNYRY

SEQ ID NO: 441 - protein, 10 aa, Unknown
  SARS-CoV sequence
RLFRKSNLKP

SEQ ID NO: 442 - protein, 8 aa, Unknown
  SARS-CoV sequence
YRLFRKSN

SEQ ID NO: 443 - protein, 8 aa, Unknown
  SARS-CoV sequence
RYRLFRKS

SEQ ID NO: 444 - protein, 9 aa, Unknown
  SARS-CoV sequence
NYNYRYRLF

SEQ ID NO: 445 - protein, 11 aa, Unknown
  SARS-CoV sequence
SKVGGNYNYRY

SEQ ID NO: 446 - protein, 9 aa, Unknown
  SARS-CoV sequence
VGGNYNYRY

SEQ ID NO: 447 - protein, 9 aa, Unknown
  SARS-CoV sequence
KVGGNYNYR

SEQ ID NO: 448 - protein, 9 aa, Unknown
  SARS-CoV sequence
YNYRYRLFR

SEQ ID NO: 449 - protein, 9 aa, Unknown
  SARS-CoV sequence
KSNLKPFER

SEQ ID NO: 450 - protein, 10 aa, Unknown
  SARS-CoV sequence
NYNYRYRLFR

SEQ ID NO: 451 - protein, 9 aa, Unknown
  SARS-CoV sequence
RLFRKSNLK

SEQ ID NO: 452 - protein, 9 aa, Unknown
  SARS-CoV sequence
GNYNYRYRL

SEQ ID NO: 453 - protein, 11 aa, Unknown
  SARS-CoV sequence
NYRYRLFRKSN

SEQ ID NO: 454 - protein, 11 aa, Unknown
  SARS-CoV sequence
YRLFRKSNLKP

SEQ ID NO: 455 - protein, 8 aa, Unknown
  SARS-CoV sequence
RLFRKSNL